SEQUENCE LISTING (56 sequences)

SEQ ID NO: 1 - protein, 12 aa, Homo sapiens
  misc_feature: spacer (IgG4hinge)
ESKYGPPCPPCP

SEQ ID NO: 2 - DNA, 36 bp, Homo sapiens
  misc_feature: spacer (IgG4hinge)
gaatctaagtacggaccgccctgccccccttgccct

SEQ ID NO: 3 - protein, 119 aa, Homo sapiens
  misc_feature: Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Hoimo sapiens
  misc_feature: Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  misc_feature: IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  misc_feature: CD28 (amino acids 153-179 of Accession No. P10747)
FWVLVVVGGVLACYSLLVTVAFIIFWV

SEQ ID NO: 9 - protein, 66 aa, Homo sapiens
  misc_feature: CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  misc_feature: CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
RSKRSRGGHSDYMNMTPRRPGPTRKHYQPYAPPRDFAAYRS

SEQ ID NO: 12 - protein, 42 aa, Homo sapiens
  misc_feature: 4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  misc_feature: CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  misc_feature: CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  misc_feature: CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - DNA, 22 bp, Artificial Sequence
  exemplary degenerate sequence of the V region of IGH
acacggcctcgtgtattactgt

SEQ ID NO: 18 - DNA, 19 bp, Artificial Sequence
  exemplary degenerate sequence of the J region of IGH
acctgaggagacggtgacc

SEQ ID NO: 19 - protein, 18 aa, Artificial sequence
  T2A
EGRGSLLTCGDVEENPGP

SEQ ID NO: 20 - protein, 22 aa, artificial sequence
  P2A
GSGATNFSLLKQAGDVEENPGP

SEQ ID NO: 21 - protein, 19 aa, Artificial Sequence
  P2A
ATNFSLLKQAGDVEENPGP

SEQ ID NO: 22 - protein, 20 aa, Artificial Sequence
  E2A
QCTNYALLKLAGDVESNPGP

SEQ ID NO: 23 - protein, 22 aa, Artificial Sequence
  F2A
VKQTLNFDLLKLAGDVESNPGP

SEQ ID NO: 24 - protein, 18 aa, Artificial Sequence
  Linker
GSTSGSGKPGSGEGSTKG

SEQ ID NO: 25 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 26 - protein, 5 aa, Artificial Sequence
  Hinge
  VARIANT (1)..(1): X = G, C, or R
  VARIANT (4)..(4): X = C or T
XPPXP

SEQ ID NO: 27 - protein, 15 aa, Homo sapiens
  MISC_FEATURE: Hinge
EPKSCDKTHTCPPCP

SEQ ID NO: 28 - protein, 12 aa, Homo sapiens
  MISC_FEATURE: Hinge
ERKCCVECPPCP

SEQ ID NO: 29 - protein, 61 aa, Homo Sapiens
  MISC_FEATURE: Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Homo sapiens
  MISC_FEATURE: Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  FMC63 CDR L1
RASQDISKYLN

SEQ ID NO: 36 - protein, 7 aa, Artificial Sequence
  FMC63 CDR L2
SRLHSGV

SEQ ID NO: 37 - protein, 9 aa, Artificial Sequence
  FMC63 CDR L3
GNTLPYTFG

SEQ ID NO: 38 - protein, 5 aa, Artificial Sequence
  FMC63 CDR H1
DYGVS

SEQ ID NO: 39 - protein, 16 aa, Artificial Sequence
  FMC63 CDR H2
VIWGSETTYYNSALKS

SEQ ID NO: 40 - protein, 7 aa, Artificial Sequence
  FMC63 CDR H3
YAMDYWG

SEQ ID NO: 41 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  SJ25C1 CDR L1
KASQNVGTNVA

SEQ ID NO: 45 - protein, 7 aa, Artificial Sequence
  SJ25C1 CDR L2
SATYRNS

SEQ ID NO: 46 - protein, 9 aa, Artificial Sequence
  SJ25C1 CDR L3
QQYNRYPYT

SEQ ID NO: 47 - protein, 5 aa, Artificial Sequence
  SJ25C1 CDR H1
SYWMN

SEQ ID NO: 48 - protein, 17 aa, Artificial Sequence
  SJ25C1 CDR H2
QIYPGDGDTNYNGKFKG

SEQ ID NO: 49 - protein, 13 aa, Artificial Sequence
  SJ25C1 CDR H3
KTISSVVDFYFDY

SEQ ID NO: 50 - protein, 122 aa, Artificial Sequence
  SJ25C1 VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  SJ25C1 VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  SJ25C1 scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  FMC63 CDR H3
HYYYGGSYAMDY

SEQ ID NO: 55 - protein, 7 aa, Artificial Sequence
  FMC63 CDR L2
HTSRLHS

SEQ ID NO: 56 - protein, 9 aa, Artificial Sequence
  FMC63 CDR L3
QQGNTLPYT

(19) United States
(12) Patent Application Publication
(10) Pub. No.: US 2020/0016199 A1
(43) Pub. Date: Jan. 16, 2020

(54) METHODS FOR THE TREATMENT OF B CELL MALIGNANCIES USING ADOPTIVE CELL THERAPY

(71) Applicants: Juno Therapeutics, Inc., Seattle, WA (US); Fred Hutchinson Canser Research Center, Seattle, WA (US)

(72) Inventors: Cameron J. TURTLE, Seattle, WA (US); David MALONEY, Seattle, WA (US); Stanley R. RIDDELL, Seattle, WA (US); Mark Gilbert, Seattle, WA (US)

(73) Assignees: Juno Therapeutics, Inc., Seattle, WA (US); Fred Hutchinson Canser Research Center, Seattle, WA (US)

(21) Appl. No.: 16/307,462

(22) PCT Filed: Jun. 6, 2017
(86) PCT No.: PCT/US2017/036231

§ 371 Date: Dec. 5, 2018

Related U.S. Application Data
(60) Provisional application No. 62/429,737, filed on Dec. 3, 2016.
(60) Provisional application No. 62/417,292, filed on Nov. 3, 2016.
(60) Provisional application No. 62/346,547, filed on Jun. 6, 2016.

(51) Int. Cl. A61K 35/17; A61P 35/00; C07K 16/28; C07K 14/725; C07K 14/705; C07K 14/71; A61K 31/664; C12Q 1/6869

(52) U.S. Cl. CPC A61K 35/17; A61P 35/00; C07K 16/2803; C07K 14/7051; C07K 14/70575; C07K 2317/622; A61K 31/664; C12Q 1/6869; C07K 2319/03; C07K 2319/02; C07K 14/71

(57) ABSTRACT

Provided are adoptive cell therapy methods involving the administration of doses of cells for treating B cell malignancies. The cells generally express recombinant receptors such as chimeric antigen receptors (CARs). In some embodiments, the methods are for treating subjects with chronic lymphocytic leukemia (CLL). In some embodiments, the methods are for treating subjects with non-Hodgkin lymphoma (NHL). In some embodiments, the methods involve prior administration of a lymphodepleting therapy, such as prior administration of fluradibine and/or another lymphodepleting chemotherapeutic agent, for example cyclophosphamide. In some embodiments, features of the methods include an increase in complete remission, overall survival and/or progression free survival of subjects treated in accord with the provided methods.

### CROSS-REFERENCE TO RELATED APPLICATIONS

This application is a U.S. National Stage of International Application No. PCT/US2017/036231 filed Jun. 6, 2017, which claims priority from U.S. provisional application No. 62/346,547, filed Jun. 6, 2016, U.S. provisional application No. 62/417,292, filed Nov. 3, 2016, and U.S. provisional application No. 62/429,737, filed Dec. 3, 2016, the contents of which are incorporated by reference in their entirety.

### STATEMENT OF GOVERNMENT INTEREST

This invention was made with government support under CA136551 awarded by the National Institutes of Health. The government has certain rights in the invention.

### INCORPORATION BY REFERENCE OF SEQUENCE LISTING

The present application is being filed along with a Sequence Listing in electronic format. The Sequence Listing is provided as a file entitled 735042006700SeqList.txt, created Dec. 3, 2018, which is 36,385 kilobytes in size. The information in the electronic format of the Sequence Listing is incorporated by reference in its entirety.

### FIELD

The present disclosure relates to adoptive cell therapy involving the administration of doses of cells for treating B cell malignancies. The cells generally express recombinant receptors such as chimeric antigen receptors (CARs). In some embodiments, the methods are for treating subjects with chronic lymphocytic leukemia (CLL). In some embodiments, the methods are for treating subjects with non-Hodgkin lymphoma (NHL). In some embodiments, the methods involve prior administration of a lymphodepleting therapy, such as prior administration of fludarabine and/or another lymphodepleting chemotherapeutic agent, for example cyclophosphamide. In some embodiments, features of the methods include an increase in complete remission, overall survival and/or progression free survival of subjects treated in accord with the provided methods.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. Improved methods are needed, for example, to increase efficacy of such methods. Provided are methods and uses that meet such needs.

### SUMMARY

Provided are methods and uses of engineered cells (e.g., T cells) and compositions thereof, for the treatment of subjects having a disease or condition, which generally is or includes a leukemia or lymphoma, most particularly a chronic lymphocytic leukemia (CLL) and/or a non-Hodgkin lymphoma (NHL). In some aspects, the methods and uses provide for or achieve improved or more durable responses or efficacy and/or a reduced risk of toxicity or other side effects, as compared to certain alternative methods, such as in particular groups of subjects treated. In some embodiments, the methods are advantageous by virtue of the administration of specified numbers or relative numbers of the engineered cells, the administration of defined ratios of particular types of the cells, the preconditioning of subjects with particular lymphodepleting therapies, treatment of particular patient populations, such as those having a particular risk profile, staging, and/or prior treatment history, and/or combinations thereof.

In some embodiments, the methods and uses include administering to the subject cells expressing genetically engineered (recombinant) cell surface receptors in adoptive cell therapy, which generally are chimeric receptors such as chimeric antigen receptors (CARs), recognizing an antigen expressed by, associated with and/or specific to the leukemia or lymphoma and/or cell type from which it is derived. The cells are generally administered in a composition formulated for administration; the methods generally involve administering one or more doses of the cells to the subject, which dose(s) may include a particular number or relative number of cells or of the engineered cells, and/or a defined ratio of two or more sub-types within the composition, such as CD4 vs CD8 T cells.

The subject generally has been preconditioned with a lymphodepleting therapy, which in some aspects increases the persistence and/or efficacy of the cells following administration, as compared to methods in which the preconditioning is not carried out or is carried out using a different lymphodepleting therapy. The lymphodepleting therapy generally includes the administration of fludarabine, typically in combination with another chemotherapy or other agent, such as cyclophosphamide, which may be administered sequentially or simultaneously in either order.

In some embodiments, the methods involve treating a subject having or suspected of having a chronic lymphocytic leukemia (CLL). In some aspects, the methods include administering to the subject a dose of cells expressing a chimeric antigen receptor (CAR) that specifically binds to a target antigen expressed by the CLL. In some aspects, the dose contains (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg. In some aspects, prior to the administration, the subject has been preconditioned with a lymphodepleting therapy that includes the administration of fludarabine.

Also provided is a method of treating a subject having or suspected of having a chronic lymphocytic leukemia (CLL), the method including administering to the subject a dose of cells expressing a chimeric antigen receptor (CAR) that specifically binds to a target antigen expressed by the CLL, said dose containing (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, wherein, prior to the administration, the subject has been preconditioned with a lymphodepleting therapy comprising the administration of fludarabine.

In some embodiments, the methods involve treating a subject having a non-Hodgkin lymphoma (NHL), such as an aggressive NHL and/or an NHL of a particular sub-type such as a diffuse large B cell lymphoma (DLBCL), a primary mediastinal large B cell lymphoma (PMBCL), a T cell/histocyte-rich large B cell lymphoma (TCHRBCL), a Burkitt's lymphoma, and/or other aggressive NHL, a mantle cell lymphoma (MCL), and/or follicular lymphoma (FL).

In some aspects, the methods include administering to the subject a dose of cells expressing a chimeric antigen receptor (CAR) that specifically binds to a target antigen expressed by the NHL. In some aspects, the dose contains (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg. The dose typically further contains a defined ratio of particular subtypes of cells, such as CD4⁺ cells expressing the CAR to CD8⁺ cells expressing the CAR and/or of CD4⁺ cells to CD8⁺ cells.

In some aspects, prior to the administration, the subject has been preconditioned with a lymphodepleting therapy that includes the administration of fludarabine.

In some aspects of any of the embodiments, the dose of cells administered includes a defined or pre-determined or engineered ratio of particular sub-types of cells. The ratio may include a ratio of CD4⁺ cells expressing the CAR to CD8⁺ cells expressing the CAR and/or of CD4⁺ cells to CD8⁺ cells. In some aspects, the CD4+ or CD8+ cells are enriched for a particular subtype, such as central-memory cells or are derived from such an enriched population, such as cells derived from CD8+ central memory cells, and/or those exhibiting increased expression of CD62L and/or CD45RO and/or CCR7 as compared to bulk T cells or bulk CD8+ T cells, or cells derived from bulk T cells or bulk CD8+ T cells. In some aspects, the ratio is or is approximately 1:1. In some aspects, it is between at or approximately 1:3 and at or approximately 3:1.

In some embodiments, at or prior to the administration of the dose of cells: the subject is or has been identified as having one or more cytogenetic abnormalities, optionally associated with high-risk NHL; the subject is or has been identified as having high-risk NHL; and/or the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL).

In some aspects of any of the embodiments, the subject is an adult and/or is over at or about 30, 40, 50, 60, or 70 years of age.

In some aspects, the lymphodepleting therapy further includes administering another chemotherapeutic agent other than the fludarabine, such as cyclophosphamide. In some aspects, the preconditioning, e.g., via the lymphodepleting therapy such as fludarabine and/or cyclohphosphamide is initiated at a time that is at least at or about 24 or at least at or about 48 hours prior to, or is between at or about 48 and at or about 96 hours prior to, the administration of the cells. In some aspects, the lymphodepleting therapy includes the administration of cyclophosphamide at or about 30-60 mg/kg of the subject (such as once daily for one, two or three days), and/or the fludarabine, such as at 25 mg/m² (e.g., daily for 2, 3, 4, or 5 or more days, such as for 3-5 days).

In some aspects of any of the embodiments, the administration of the cell dose and/or the lymphodepleting therapy is carried out via outpatient delivery.

In some embodiments, at or prior to the administration of the dose of cells, the subject is or has been identified as having one or more cytogenetic abnormalities and/or other risk factors. In some aspects, the abnormalities or factor(s) are associated with high-risk or very-high risk disease, such as high-risk or very high risk CLL and/or NHL.

Such factors may be those detected or detectable by FISH and/or those not detectable by FISH. The abnormalities may include complex karyotype (CK), translocations, deletion of the long arm of chromosome 13 (del 13q), del 11, trisomy 12, del 17p, del 6q, and del 13q.14, optionally as detected by FISH. In some aspects, the abnormalities include CK and/or del17p. In some aspects, the subject treated by the methods and uses is or has been identified as having high-risk CLL or very high-risk CLL and optionally is selected for the treatment based on such classification and/or a particular abnormality. In some aspects of any of the embodiments, the subject is or has been identified as, and/or is selected for, having metastatic, aggressive, advanced, and/or extramedullary form of the disease or condition; and/or the subject is an adult and/or is over at or about 30, 40, 50, 60, or 70 years of age.

In some embodiments, prior to the administration of the dose of cells, the subject has been treated with two or more, optionally 3, 4, 5, 6, 7, 8, or 9 more, therapies for the leukemia or lymphoma, such as the NHL and/or the CLL and/or with two or more, optionally 3, 4, 5, 6, 7, 8, or 9 more, therapies other than the lymphodepleting therapy and/or other than another dose of cells expressing the CAR. In some embodiments, prior to the administration of the dose of cells, the subject has been treated for the CLL with a kinase inhibitor, optionally an inhibitor of Btk, optionally ibrutinib, and/or a biologic and/or immunotherapy, such as monoclonal antibody. In some embodiments, prior to the administration of the dose of cells, the subject has been treated with two or more, optionally 2, 3, or 4 more, therapies for the leukemia or lymphoma, which generally are therapies other than the lymphodepleting therapy and/or the cells or another dose of cells expressing the CAR, optionally other than cells expressing a different CAR.

In some embodiments, at or immediately prior to the time of the administration of the dose of cells, the subject has relapsed following remission after treatment with, or become refractory to, such one or more prior therapies.

In some embodiments, prior to the administration of the dose of cells, the subject has been treated for the leukemia or lymohoma (such as the CLL or NHL) with a monoclonal antibody such as one that specifically binds to an antigen expressed by, or previously expressed by, cells of the CLL or the NHL. In some embodiments, at or immediately prior to the time of the administration of the dose of cells, the subject has relapsed following remission after treatment with, or become refractory to, one or more prior therapies for the CLL.

In some embodiments, the method further includes, prior to the administration of the cell dose, administering the lymphodepleting therapy to the subject.

In some embodiments, the dose of cells contains a defined ratio of CD4+ cells expressing the CAR to CD8+ cells expressing the CAR and/or of CD4+ cells to CD8+ cells, which optionally is approximately 1:1 or is between approximately 1:3 and approximately 3:1. In some embodiments, the dose of cells is administered parenterally, optionally intravenously.

In some embodiments of the methods provided herein, at least 50% of subjects treated according to the method achieve complete remission (CR) and/or objective response (OR); and/or the subject exhibits CR, OR, lymph nodes of less than at or about 20 mm in size, within 1 month of the administration of the dose of cells; and/or a malignant immunoglobulin heavy chain (IGH) locus and/or an index clone of the disease or condition such as the CLL or NHL is not detected in the bone marrow of the subject (or in the bone marrow of greater than 50% of subjects treated according to the methods), optionally as assessed by IGH deep sequencing, optionally at a time that is at or about or at least at or about 1, 2, 3, 4, 5, 6, 12, 18, or 24 months following the administration of the cell dose.

In some embodiments of the methods provided herein, at least 50% of subjects that are treated according to the method, and that achieve complete remission (CR), exhibit progression-free survival (PFS) and/or overall survival (OS) of greater than 12 months; on average, subjects treated according to the method exhibit a median PFS or OS of greater than at or about 6 months, 12 months, or 18 months; and/or the subject exhibits PFS or OS following therapy for at least at or about 6, 12, 18 or more months.

In some embodiments, the antigen is a B cell antigen. In some aspects, the antigen is CD19. In some aspects, the antigen is or includes CD20, CD22, CD30, CD33, CD38, ROR1, or other marker associated with B cells or B cell cancer.

In some embodiments, the CAR contains a binding domain, which typically is or comprises an scFv specific for the antigen, a transmembrane domain, and one or more cytoplasmic signaling domains or regions, which may be derived from natural or endogenous signaling molecules or functional variants thereof. In some aspects, the signaling region includes domains capable of delivering primary and secondary signals to a T cell or other immune cell. The domains may include those derived from a costimulatory molecule, such as from a 4-1BB, e.g., a human 4-1BB, and/or a CD28 molecule, such as a human CD28. The domains may further include a cytoplasmic signaling domain derived from a primary signaling ITAM-containing molecule, such as a CD3zeta, e.g., human CD3zeta. In some embodiments, the CAR contains a spacer and/or hinge region, which may in some aspects be derived from a human IgG.

In some embodiments, at least at or about 50%, at least at or about 60%, at least at or about 70%, at least at or about 80%, or at least at or about 90%, of subjects treated according to the method achieve complete remission (CR), such as measured by RECIST criteria and/or Lugano criteria, and/or any of a number of known criteria for assessing response. In some embodiments, at least at or about 50%, at least at or about 60%, at least at or about 70%, at least at or about 80%, or at least at or about 90%, of subjects treated according to the method achieve objective response (OR), such as measured by RECIST criteria and/or Lugano criteria, and/or any of a number of known criteria for assessing response. In some embodiments, the subject, or at least at or about 50%, at least at or about 60%, at least at or about 70%, at least at or about 80%, or at least at or about 90%, of subjects that are treated according to the method, and that achieve complete remission (CR), exhibit progression-free survival (PFS) and/or overall survival (OS) of greater than 12, 13, 14, 15, 16, 17, 18, 19, 20, 21, 22, 23, 24, or more months, e.g., on average, and/or greater than 6, 12, or 18 months. In some embodiments, the subject, or at least at or about 50%, at least at or about 60%, at least at or about 70%, at least at or about 80%, or at least at or about 90%, of subjects treated according to the method, exhibit(s) a median PFS or OS of greater than at or about 6 months, 12 months, or 18 months; and/or exhibits PFS or OS following therapy for at least at or about 6, 12, 18, or 24, or more months.

In some embodiments, the subject does not exhibit grade 3 or higher neurotoxicity and/or does not exhibit severe CRS, or does not do so within a certain period of time following the treatment, such as within a week, two weeks, or one month of the administration of the cells.

Provided is method of treating a subject having a non-Hodgkin lymphoma (NHL), the method including administering to the subject a dose of cells expressing a chimeric antigen receptor (CAR) that specifically binds to a target antigen expressed by the NHL, wherein said dose (i) contains (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg, and (ii) contains a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1 wherein, prior to the administration, the subject has been preconditioned with a lymphodepleting therapy comprising the administration of fludarabine.

Also provided is a method of treating a subject having a non-Hodgkin lymphoma (NHL), the method including administering to the subject a dose of cells expressing a chimeric antigen receptor (CAR) that specifically binds to a target antigen expressed by the NHL, wherein said dose (i) contains (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, and (ii) contains a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1, wherein, prior to the administration, the subject has been preconditioned with a lymphodepleting therapy comprising the administration of fludarabine.

In some embodiments, at or prior to the administration of the dose of cells, the subject is or has been identified as having one or more cytogenetic abnormalities, optionally associated with high-risk NHL; the subject is or has been identified as having high-risk NHL; and/or the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or the subject is an adult and/or is over at or about 30, 40, 50, 60, or 70 years of age.

In some of any such embodiments, wherein, prior to the administration of the dose of cells, the subject has been treated with two or more, optionally 2, 3, or 4 or more, therapies for the NHL other than the lymphodepleting therapy and/or other than another dose of cells expressing the CAR. In some of any such embodiments, at or immediately prior to the time of the administration of the dose of cells, the subject has relapsed following remission after treatment with, or become refractory to, one or more prior therapies for the NHL.

In some of any such embodiments, the method further includes prior to the administration of the cell dose, administering the lymphodepleting therapy to the subject. In some embodiments, the lymphodepleting therapy (i) further includes administering another chemotherapeutic agent other than the fludarabine, which optionally is cyclophosphamide; (ii) is initiated at a time that is at least at or about 48 hours prior to or is between at or about 48 and at or about 96 hours prior to the administration of the cells; and (iii) includes the administration of cyclophosphamide at about 30-60 mg/kg, optionally once daily for one or two days, and/or the fludarabine at about 25 mg/m², daily for 3-5 days. In some of any such embodiments, the administration of the cell dose and/or the lymphodepleting therapy is carried out via outpatient delivery.

In some of any such embodiments, the defined ratio is a defined ratio of CD4+ cells expressing the CAR to CD8+ cells expressing the CAR of at or about 1:1 and/or is a defined ratio of CD4+ cells to CD8+ cells, which is at or about 1:1. In some embodiments, the dose of cells is administered parenterally, optionally intravenously.

In some of any such embodiments, at least 50% of subjects treated according to the method achieve complete remission (CR) and/or objective response (OR). In some embodiments of the method, at least 50% of subjects that are treated according to the method, and that achieve complete remission (CR), exhibit progression-free survival (PFS) and/or overall survival (OS) of greater than 12 months; on average, subjects treated according to the method exhibit a median PFS or OS of greater than at or about 6 months, 12 months, or 18 months; and/or the subject exhibits PFS or OS following therapy for at least at or about 6, 12, 18 or more months.

In some of any such embodiments, the antigen is a B cell antigen, which optionally is CD19. In some embodiments, the CAR contains an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta. In some cases, the CAR contains a spacer and/or hinge region, each optionally derived from a human IgG.

In some of any such embodiments, the CAR contains, in order, an scFv specific for the antigen, a transmembrane domain, a cytoplasmic signaling domain derived from a costimulatory molecule, which optionally is or includes a 4-1BB signaling domain, and a cytoplasmic signaling domain derived from a primary signaling ITAM-containing molecule, which optionally is a CD3zeta signaling domain; or the CAR contains, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain; and wherein the spacer is optionally a polypeptide spacer that (a) contains or consists of all or a portion of an immunoglobulin hinge or a modified version thereof or contains about 15 amino acids or less, and does not contain a CD28 extracellular region or a CD8 extracellular region, (b) contains or consists of all or a portion of an immunoglobulin hinge, optionally an IgG4 hinge, or a modified version thereof and/or contains about 15 amino acids or less, and does not contain a CD28 extracellular region or a CD8 extracellular region, or (c) is at or about 12 amino acids in length and/or contains or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) includes or consists of the formula X1PPX2P, where X1 is glycine, cysteine or arginine and X2 is cysteine or threonine; and/or the costimulatory domain contains SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain contains SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv contains a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv includes a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv contains, in order, a VH, a linker, optionally comprising SEQ ID NO: 24, and a VL, and/or the scFv contains a flexible linker and/or includes the amino acid sequence set forth as SEQ ID NO: 24.

Provided is a method of prognosis or staging, the method including detecting the presence or absence of a malignant immunoglobulin heavy chain locus (IGH) sequence in a sample from a subject having a B cell malignancy, said subject having previously received administration of a cell therapy comprising a dose or composition of genetically engineered cells expressing a recombinant receptor for treating the B cell malignancy, wherein detecting the presence or absence of the malignant IGH sequence determines the prognosis of the subject in response to the cell therapy. In some aspects, the detecting the presence or absence of the malignant IGH sequence is carried out within or within about or about 3 to 6 weeks after initiation of the cell therapy, optionally within or within about 4 weeks of initiation of administration of the cell therapy.

In some of any such embodiments, if the malignant IGH sequence is detected, the subject is identified as not responding or not exhibiting a complete response (CR) or an overall response (OR) to the cell therapy or as likely to relapse to the cell therapy. In some embodiments, if the malignant IGH sequence is detected identifying the subject as a candidate for further treatment and/or for receiving an altered or alternative treatment. In some embodiments, if the malignant IGH sequence is detected discontinuing administration of the cell therapy, administering to the subject a further dose of the cell therapy, administering to the subject a higher dose of the cell therapy, administering o the subject a different cell therapy, optionally a cell therapy expressing a different recombinant receptor, and/or administering to the subject an alternative therapeutic agent for treating the B cell malignancy.

In some of any such embodiments, if the malignant IGH sequence is not detected, the subject is identified as responding to the cell therapy and/or as exhibiting a complete response (CR) or overall response (OR) to the cell therapy or as likely not to relapse to the cell therapy. In some embodiments, if the malignant IGH sequence is not detected, the subject is identified as a candidate for no further treatment and/or is not further treated, optionally is not further treated with the cell therapy and/or is not further treated with an alternative therapy for the B cell malignancy.

Provided is a method of predicting durability of response to a cell therapy, the method including detecting the presence or absence of a malignant immunoglobulin heavy chain locus (IGH) sequence in a sample from a subject having a B cell malignancy, said subject having previously received administration of a cell therapy including a dose or composition of genetically engineered cells expressing a recombinant receptor for treating the B cell malignancy, wherein the presence or absence of the malignant IGH sequence predicts the durability of response to the cell therapy. In some aspects, the detecting the presence or absence of the malignant IGH sequence is carried out within or within about or about 4 weeks, 6 weeks, 8 weeks, 12 weeks or 16 weeks after initiation of the cell therapy.

In some of any such embodiments, if the malignant IGH sequence is not detected, the subject is predicted to exhibit or likely to exhibit a durable response to the cell therapy and/or to be at a low or relatively low risk of relapse within a certain period of time and/or to have a high likelihood of exhibiting progression free survival for at least a certain period of time. In some embodiments, if the malignant IGH sequence is not detected, the subject is predicted to exhibit survival without progression for greater than or about 3 months, greater than about 6 months, greater than about 9 months or greater than about 12 months after initiation of the cell therapy; and/or to remain surviving for greater than or greater than about 3 months, greater than or greater than about 6 months, greater than or greater than about 9 months or greater than about 12 months after initiation of the cell therapy; and/or to exhibit durable CR or OR for greater than or greater than about 3 months, greater than or greater than about 6 months or greater than or greater than about 9 months after initiation of the cell therapy; and/or not likely to relapse following initiation of administration of the cell therapy, optionally not likely to relapse within 3 months, 6 months or 9 months after initiation of administration of the cell therapy.

In some of any such embodiments, if the malignant IGH sequence is detected, the subject is predicted to exhibit or likely to exhibit a response to the cell therapy that is not durable and/or to be at a high or relatively high risk of relapse within a certain period of time and/or to have a low likelihood of exhibiting progression free survival for at least a certain period of time. In some embodiments, if the malignant IGH sequence is not detected, the subject is s predicted not to exhibit survival without progression for greater than or about 3 months, greater than about 6 months, greater than about 9 months or greater than about 12 months after initiation of the cell therapy; and/or not to remain surviving for greater than or greater than about 3 months, greater than or greater than about 6 months, greater than or greater than about 9 months or greater than about 12 months after initiation of the cell therapy; and/or not to exhibit durable CR or OR for greater than or greater than about 3 months, greater than or greater than about 6 months or greater than or greater than about 9 months after initiation of the cell therapy.

In some of any such embodiments, if the malignant IGH sequence is detected administering to the subject a further dose of the cell therapy, administering to the subject a higher dose of the cell therapy, administering o the subject a different cell therapy, optionally a cell therapy expressing a different recombinant receptor, and/or administering to the subject an alternative therapeutic agent for treating the B cell malignancy. In some embodiments, the presence or absence of the malignant IGH sequence is determined by IGH sequencing, optionally comprising PCR amplification of IGH target DNA.

In some of any such embodiments, the sample contains B cells. In some embodiments, the sample contains a blood or bone marrow sample. In some aspects, the sample has been obtained from the subject. In some of any such embodiments, the method is carried out ex vivo.

In some of any such embodiments, the B cell malignancy is a cancer. In some cases, the B cell malignancy is or includes a leukemia. In some examples, the B cell malignancy is an antigen or is associated with an antigen selected from CD19, CD20, CD22, CD30, CD33 or CD38, ROR1. In some embodiments, the B cell malignancy is selected from and/or is acute lymphoblastic leukemia (ALL), adult ALL, chronic lymphoblastic leukemia (CLL), non-Hodgkin lymphoma (NHL), and Diffuse Large B-Cell Lymphoma (DLBCL). In some examples, the B cell malignancy is or includes chronic lymphoblastic leukemia (CLL) or high-risk CLL. In some instances, the B cell malignancy is or includes non-Hodgkin lymphoma (NHL). In some aspects, the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some of any such embodiments, the recombinant receptor specifically binds to an antigen associated with the disease or condition or expressed in cells of the environment of a lesion associated with the B cell malignancy. In some embodiments, the recombinant receptor is a T cell receptor or a functional non-T cell receptor. In some cases, the recombinant receptor is a chimeric antigen receptor (CAR). In some instances, the CAR contains an extracellular antigen-recognition domain that specifically binds to the antigen and an intracellular signaling domain comprising an ITAM, wherein optionally, the intracellular signaling domain includes an intracellular domain of a CD3-zeta (CD3) chain; and/or wherein the CAR further includes a costimulatory signaling region, which optionally includes a signaling domain of CD28 or 4-1BB. In some embodiments, the CAR contains an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta. In some embodiments, the CAR contains a spacer and/or hinge region, each optionally derived from a human IgG.

In some of any such embodiments, the CAR contains, in order, an scFv specific for the antigen, a transmembrane domain, a cytoplasmic signaling domain derived from a costimulatory molecule, which optionally is or includes a 4-1BB signaling domain, and a cytoplasmic signaling domain derived from a primary signaling ITAM-containing molecule, which optionally is a CD3zeta signaling domain; or the CAR contains, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain; and wherein the spacer is optionally a polypeptide spacer that (a) contains or consists of all or a portion of an immunoglobulin hinge or a modified version thereof or contains about 15 amino acids or less, and does not include a CD28 extracellular region or a CD8 extracellular region, (b) contains or consists of all or a portion of an immunoglobulin hinge, optionally an IgG4 hinge, or a modified version thereof and/or includes about 15 amino acids or less, and does not include a CD28 extracellular region or a CD8 extracellular region, or (c) is at or about 12 amino acids in length and/or includes or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) contains or consists of the formula X1PPX2P, where X1 is glycine, cysteine or arginine and X2 is cysteine or threonine; and/or the costimulatory domain includes SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain includes SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv contains a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv includes a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv includes, in order, a VH, a linker, optionally comprising SEQ ID NO: 24, and a VL, and/or the scFv includes a flexible linker and/or includes the amino acid sequence set forth as SEQ ID NO: 24.

In some of any such embodiments, the engineered cells contains T cells, optionally CD4+ and/or CD8+. In some cases, the T cells are primary T cells obtained from a subject. In some embodiments, the engineered cells are autologous to the subject. In some cases, the engineered cells are allogeneic to the subject.

Provided is an article of manufacture containing one or more dose of a cell therapy, each dose comprising cells expressing a chimeric antigen receptor (CAR), and instructions for administering the cell therapy, wherein the instructions specify the dose of cells is to be administered to a subject having a chronic lymphocytic leukemia (CLL); and the instructions specify administration of a number of CAR-expressing or a number of cells, or specify administration of an amount or volume of one or more formulations corresponding to or containing said specified number of cells, wherein the specified number of cells to be administered contains a number to administer a dose of cells comprising (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg.

Provided is an article of manufacture comprising one or more dose of a cell therapy, each dose containing cells expressing a chimeric antigen receptor (CAR), and instructions for administering the cell therapy, wherein the instructions specify the dose of cells is to be administered to a subject having a chronic lymphocytic leukemia (CLL); and the instructions specify administration of a number of CAR-expressing or a number of cells, or specify administration of an amount or volume of one or more formulations corresponding to or containing said specified number of cells, wherein the specified number of cells to be administered contains a number to administer a dose of cells comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.

In some embodiments of the article of manufacture, further included are instructions for use with, after or in connection with a lymphodepleting therapy, the lympodepleting therapy comprising fludarabine. In some cases, the instructions specify that the cell therapy is to be administer to a subject that is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH; is or has been identified as having high-risk CLL; and/or is or has been identified as having extramedullary disease; and/or is or has been identified as having central nervous system (CNS) disease; and/or is an adult and/or is over at or about 30, 40, 50, 60, or 70 years of age.

In some embodiments, the instructions specify that the cell therapy is to be administered to a subject that has been treated with two or more, optionally 3, 4, 5, 6, 7, 8, or 9 or more, therapies for the CLL, other than the lymphodepleting therapy and/or other than another dose of cells expressing the CAR; and/or has been treated for the CLL with a kinase inhibitor, optionally an inhibitor of Btk, optionally ibrutinib; and/or has been treated for the CLL with a monoclonal antibody that specifically binds to an antigen expressed by, or previously expressed by, cells of the CLL; and/or has been treated for the CLL with venetoclax, a combination therapy comprising fludarabine and rituximab, radiation therapy and/or hematopoietic stem cell transplantation (HSCT).

In some of any such embodiments, the instructions specify that the cell therapy is to be administered to a subject that has relapsed following remission after treatment with, or become refractory to, one or more prior therapies for the CLL.

Provided is an article of manufacture containing one or more dose of a cell therapy, each dose containing cells expressing a chimeric antigen receptor (CAR), and instructions for administering the cell therapy, wherein the instructions specify the dose of cells is to be administered to a subject having a non-Hodgkin lymphoma (NHL); and the instructions specify administration of a number of CAR-expressing or a number of cells, or specify administration of an amount or volume of one or more formulations corresponding to or containing said specified number of cells, wherein the specified number of cells to be administered contains a number to administer a dose of cells containing (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg.

Provided is an article of manufacture containing one or more dose of a cell therapy, each dose comprising cells expressing a chimeric antigen receptor (CAR), and instructions for administering the cell therapy, wherein the instructions specify the dose of cells is to be administered to a subject having a non-Hodgkin lymphoma (NHL); and the instructions specify administration of a number of CAR-expressing or a number of cells, or specify administration of an amount or volume of one or more formulations corresponding to or containing said specified number of cells, wherein the specified number of cells to be administered contains a number to administer a dose of cells containing (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells. In some embodiments, the article of manufacture further contains instructions for use with, after or in connection with a lymphodepleting therapy, the lympodepleting therapy comprising fludarabine.

In some of any such embodiments, the instructions specify that the cell therapy is to be administer to a subject that is or has been identified as having one or more cytogenetic abnormalities, optionally associated with high-risk NHL; is or has been identified as having high-risk NHL; and/or is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or is an adult and/or is over at or about 30, 40, 50, 60, or 70 years of age.

In some of any such embodiments, the instructions specify that the cell therapy is to be administered to a subject that has been treated with two or more, optionally 2, 3 or 4 or more, therapies for the NHL, other than the lymphodepleting therapy and/or other than another dose of cells expressing the CAR. In some embodiments, the instructions specify that the cell therapy is to be administered to a subject that has relapsed following remission after treatment with, or become refractory to, one or more prior therapies for the NHL.

In some of any such embodiments, the lymphodepleting therapy (i) further includes administering another chemotherapeutic agent other than the fludarabine, which optionally is cyclophosphamide; and/or (ii) includes the administration of cyclophosphamide at about 30-60 mg/kg, optionally once daily for one or two days, and/or the fludarabine at about 25 mg/m², daily for 3-5 days. In some embodiments, the instructions specify that the lympodepleting therapy is initiated at a time that is at least at or about 48 hours prior to or is between at or about 48 and at or about 96 hours prior to the administration of the cell therapy.

In some of any such embodiments, the instructions specify administering the cell therapy at a defined ratio of CD4⁺ cells expressing the CAR to CD8⁺ cells, or specify administering amounts of volumes of the formulation(s) corresponding to such defined ratio, or includes a formulation having the cells at such ratio or contains the cells at such ratio expressing the CAR and/or of CD4⁺ cells to CD8⁺ cells, which ratio optionally is approximately 1:1 or is between approximately 1:3 and approximately 3:1. In some of any such embodiments, the instructions further specify the cell therapy is for parenteral administration, optionally intravenous administration. In some of any such embodiments, the instructions further specify the administration of the cell therapy is to be or may be administered to the subject on an outpatient setting and/or without admission of the subject to the hospital overnight or for one or more consecutive days and/or is without admission of the subject to the hospital for one or more days.

In some of any such embodiments, the cell therapy contains primary T cells obtained from a subject. In some cases, the T cells are autologous to the subject. In some cases, the T cells are allogeneic to the subject.

In some of any such embodiments, the CAR contains an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta. In some embodiments, the CAR contains a spacer and/or hinge region, each optionally derived from a human IgG. In some embodiments, the antigen is a B cell antigen, which optionally is CD19.

In some of any such embodiments, the CAR contains, in order, an scFv specific for the antigen, a transmembrane domain, a cytoplasmic signaling domain derived from a costimulatory molecule, which optionally is or contains a 4-1BB signaling domain, and a cytoplasmic signaling domain derived from a primary signaling ITAM-containing molecule, which optionally is a CD3zeta signaling domain; or the CAR contains, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain; and wherein the spacer is optionally a polypeptide spacer that (a) includes or consists of all or a portion of an immunoglobulin hinge or a modified version thereof or includes about 15 amino acids or less, and does not include a CD28 extracellular region or a CD8 extracellular region, (b) contains or consists of all or a portion of an immunoglobulin hinge, optionally an IgG4 hinge, or a modified version thereof and/or contains about 15 amino acids or less, and does not include a CD28 extracellular region or a CD8 extracellular region, or (c) is at or about 12 amino acids in length and/or contains or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) includes or consists of the formula X1PPX2P, where X1 is glycine, cysteine or arginine and X2 is cysteine or threonine; and/or the costimulatory domain contains SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain contains SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv contains a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv includes a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv includes, in order, a VH, a linker, optionally comprising SEQ ID NO: 24, and a VL, and/or the scFv includes a flexible linker and/or includes the amino acid sequence set forth as SEQ ID NO: 24.

Provided is a composition containing cells expressing a chimeric antigen receptor (CAR) that specifically binds to a target antigen of a chronic lymphocytic leukemia (CLL) for use in treating a subject having or suspected of having CLL, wherein the treating includes administering to the subject a dose of cells expressing the CAR, said dose containing (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg, wherein, prior to the administration, the subject has been preconditioned with a lymphodepleting therapy comprising the administration of fludarabine.

Provided is a composition containing cells expressing a chimeric antigen receptor (CAR) that specifically binds to a target antigen of a chronic lymphocytic leukemia (CLL) for use in treating a subject having or suspected of having CLL, wherein the treating includes administering to the subject a dose of cells expressing the CAR, said dose comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, wherein, prior to the administration, the subject has been preconditioned with a lymphodepleting therapy comprising the administration of fludarabine.

In some embodiments of the use of the compositions described, the composition is for use in treating a subject in which, at or prior to the administration of the dose of cells: the subject is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH; the subject is or has been identified as having high-risk CLL; and/or the subject is or has been identified as having extramedullary disease; and/or the subject is or has been identified as having central nervous system (CNS) disease; and/or the subject is an adult and/or is over at or about 30, 40, 50, 60, or 70 years of age.

In some of any such embodiments, the composition is for use in treating a subject in which, prior to the administration of the dose of cells, the subject has been treated with two or more, optionally 3, 4, 5, 6, 7, 8, or 9 or more, therapies for the CLL, other than the lymphodepleting therapy and/or other than another dose of cells expressing the CAR. In some of any such embodiments, the composition is for use in treating a subject in which, prior to the administration of the dose of cells, the subject has been treated with two or more, optionally 3, 4, 5, 6, 7, 8, or 9 or more, therapies for the CLL, other than another dose of cells expressing the CAR or other than another dose of cells expressing the CAR and the preconditioning therapy.

In some of any such embodiments, the composition is for use in treating a subject in which, prior to the administration of the dose of cells, the subject has been treated for the CLL with a kinase inhibitor, optionally an inhibitor of Btk, optionally ibrutinib. In some embodiments, the composition is for use in treating a subject in which, prior to the administration of the dose of cells, the subject has been treated for the CLL with a monoclonal antibody that specifically binds to an antigen expressed by, or previously expressed by, cells of the CLL.

In some of any such embodiments, the composition is for use in treating a subject in which, prior to the administration of the dose of cells, the subject has been treated for the CLL with venetoclax, a combination therapy comprising fludarabine and rituximab, radiation therapy and/or hematopoietic stem cell transplantation (HSCT). In some aspects, the composition is for use in treating a subject in which, at or immediately prior to the time of the administration of the dose of cells, the subject has relapsed following remission after treatment with, or become refractory to, one or more prior therapies for the CLL.

Provided is a composition containing cells expressing a chimeric antigen receptor (CAR) that specifically binds to a target antigen of a non-Hodgkin lymphoma (NHL) for use in treating a subject having or suspected of having NHL, wherein the treating includes administering to the subject a dose of cells expressing a chimeric antigen receptor (CAR) that specifically binds to a target antigen expressed by the NHL, wherein the treating includes administering to the subject a dose of cells expressing the CAR, said dose (i) contains (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg, and (ii) contains a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1, wherein, prior to the administration, the subject has been preconditioned with a lymphodepleting therapy comprising the administration of fludarabine.

Provided is a composition containing cells expressing a chimeric antigen receptor (CAR) that specifically binds to a target antigen of a non-Hodgkin lymphoma (NHL) for use in treating a subject having or suspected of having NHL, wherein the treating includes administering to the subject a dose of cells expressing the CAR, said dose (i) contains (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, and (ii) contains a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1, wherein, prior to the administration, the subject has been preconditioned with a lymphodepleting therapy comprising the administration of fludarabine.

In some embodiments, the composition is for use in treating a subject in which, at or prior to the administration of the dose of cells, the subject is or has been identified as having one or more cytogenetic abnormalities, optionally associated with high-risk NHL; the subject is or has been identified as having high-risk NHL; and/or the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or the subject is an adult and/or is over at or about 30, 40, 50, 60, or 70 years of age.

In some of any such embodiments, the composition is for use in treating a subject in which, prior to the administration of the dose of cells, the subject has been treated with two or more, optionally 2, 3, or 4 or more, therapies for the NHL other than the lymphodepleting therapy and/or other than another dose of cells expressing the CAR. In some cases, the composition is for use in treating a subject in which, at or immediately prior to the time of the administration of the dose of cells, the subject has relapsed following remission after treatment with, or become refractory to, one or more prior therapies for the NHL.

In some of any such embodiments, the lymphodepleting therapy (i) further includes administration of another chemotherapeutic agent other than the fludarabine, which optionally is cyclophosphamide; (ii) is initiated at a time that is at least at or about 48 hours prior to or is between at or about 48 and at or about 96 hours prior to the administration of the cells; and (iii) includes the administration of cyclophosphamide at about 30-60 mg/kg, optionally once daily for one or two days, and/or the fludarabine at about 25 mg/m², daily for 3-5 days. In some embodiments, the treating includes administration of the cell dose and/or the lymphodepleting therapy via outpatient delivery.

In some of any such embodiments, the composition and/or the dose of cells contains a defined ratio of CD4+ cells expressing the CAR to CD8+ cells expressing the CAR and/or of CD4+ cells to CD8+ cells, which optionally is approximately 1:1 or is between approximately 1:3 and approximately 3:1. In some of any such embodiments, the composition and/or dose of cells is formulated for parenteral administration, optionally intravenous administration.

In some embodiments, the antigen is a B cell antigen, which optionally is CD19. In some of any such embodiments, the CAR contains an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta. In some cases, the CAR contains a spacer and/or hinge region, each optionally derived from a human IgG.

In some of any such embodiments, the CAR contains, in order, an scFv specific for the antigen, a transmembrane domain, a cytoplasmic signaling domain derived from a costimulatory molecule, which optionally is or includes a 4-1BB signaling domain, and a cytoplasmic signaling domain derived from a primary signaling ITAM-containing molecule, which optionally is a CD3zeta signaling domain; or the CAR contains, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or includes a CD3zeta signaling domain; and wherein the spacer is optionally a polypeptide spacer that (a) contains or consists of all or a portion of an immunoglobulin hinge or a modified version thereof or contains about 15 amino acids or less, and does not include a CD28 extracellular region or a CD8 extracellular region, (b) contains or consists of all or a portion of an immunoglobulin hinge, optionally an IgG4 hinge, or a modified version thereof and/or includes about 15 amino acids or less, and does not include a CD28 extracellular region or a CD8 extracellular region, or (c) is at or about 12 amino acids in length and/or includes or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) includes or consists of the formula X1PPX2P, where X1 is glycine, cysteine or arginine and X2 is cysteine or threonine; and/or the costimulatory domain includes SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain includes SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv includes a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv contains a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv contains, in order, a VH, a linker, optionally comprising SEQ ID NO: 24, and a VL, and/or the scFv contains a flexible linker and/or contains the amino acid sequence set forth as SEQ ID NO: 24.

### BRIEF DESCRIPTION OF THE DRAWINGS

FIG. 1A shows percent progression-free survival (PFS) curves for relapsed or refractory (R/R) CD19⁺ chronic lymphocytic leukemia (CLL) subjects treated with a single infusion of 2×10⁵, 2×10⁶ or 2×10⁷ CAR-expressing T cells per kilogram (kg) weight of the subject. Separate curves are shown for subjects who achieved complete remission (CR) and for subjects that did not (non CR). Prior to the infusion, subjects were pre-conditioned with 60 mg/kg cyclophosphamide and 25 mg/m² fludarabine (Flu) daily for 3-5 days.

FIG. 1B shows percent overall survival (OS) curves for relapsed or refractory (R/R) CD19⁺ chronic lymphocytic leukemia (CLL) subjects treated with a single infusion of 2×10⁵, 2×10⁶ or 2×10⁷ CAR-expressing T cells per kilogram (kg) weight of the subject. Separate curves are shown for subjects who achieved complete remission (CR) and for subjects that did not (non CR). Prior to the infusion, subjects were pre-conditioned with 60 mg/kg cyclophosphamide and 25 mg/m² fludarabine (Flu) daily for 3-5 days.

FIG. 2A shows percent progression-free survival (PFS) curves for non-Hodgkin lymphoma (NHL) subjects treated with a single infusion of 2×10⁶ CAR-expressing T cells per kilogram (kg) weight of the subject. Separate curves are shown for subjects who achieved complete remission (CR) and for subjects that did not (non CR). Prior to the infusion, subjects were pre-conditioned with 60 mg/kg cyclophosphamide and 25 mg/m² fludarabine (Flu) daily for 3-5 days.

FIG. 2B shows percent overall survival (OS) curves for non-Hodgkin lymphoma (NHL) subjects treated with a single infusion of 2×10⁶ CAR-expressing T cells per kilogram (kg) weight of the subject. Separate curves are shown for subjects who achieved complete remission (CR) and for subjects that did not (non CR). Prior to the infusion, subjects were pre-conditioned with 60 mg/kg cyclophosphamide and 25 mg/m² fludarabine (Flu) daily for 3-5 days.

FIG. 3 shows percent progression-free survival (PFS) curves for relapsed or refractory (R/R) CD19⁺ chronic lymphocytic leukemia (CLL) subjects treated with a single infusion of 2×10⁵, 2×10⁶ or 2×10⁷ CAR-expressing T cells per kilogram (kg) weight of the subject and pre-conditioned with 30-60 mg/kg cyclophosphamide and 25 mg/m² fludarabine (Flu) daily for 3-5 days (n=13). Separate curves are shown for subjects who achieved complete remission (CR) and for subjects that did not (non CR).

FIG. 4A-E shows correlation of CAR-T cell expansion with the percentage of abnormal B cells present in bone marrow (A), the tumor cross-sectional area (B), the absolute abnormal B cell count in blood (C), the maximum SUV on PET imaging (D) and the immune checkpoint biomarker CD200 (E) for patients treated with Cy/Fu and 2×10⁶ CAR-T cells/kg.

FIG. 5 shows the overall survival (OS) and progression-free survival (PFS) for all CLL patients in the study. The median follow-up is 12.4 months.

FIG. 6A and FIG. 6B show progression free survival (PFS) and overall survival (OS) in patients as measured by IWCLL 2008 nodal response criteria, after Cy/Flu lymphodepletion and CAR-T cell infusion at 2×10⁵ or 2×10⁶ CAR-T cells/kg. FIG. 6A shows PFS and OS in patients with CR, PR or that were non-responders (n=20). FIG. 6B shows PFS and OS in patients with CR, PR or no response (SD/PD), which excluded one patient in FIG. 6A that died prior to restaging. For both FIGS. 6A and 6B, separate curves are shown for the group of subjects who achieved CR, subjects who achieved PR, and non-responders. The median PFS and OS follow-up for patients in CR/PR is 12.3 and 12.4 months, respectively. mPFS, median PFS; mOS, median OS; NR, not reached.

FIG. 7 shows percent progression-free survival (PFS) and overall-survival (OS) curves for fourteen subjects that achieved complete remission (CR) from bone marrow 4 weeks after CAR− T cell infusion by flow cytometry and had no detectable malignant IGH copies (IGHseq-negative) compared to those who had detectable malignant IGH copies (IGHseq-positive).

FIG. 8 shows CD4⁺/EGFRt⁺ and CD8⁺/EGFRt⁺ CAR-T cell counts for fourteen subjects that achieved complete remission (CR) and had IGH deep sequencing of bone marrow performed.

FIG. 9A shows the peak CD4+/EGFRt+ (left) and CD8+/EGFRt+ (right) CAR-T cell counts in blood from patients who did (Yes) or did not (No) clear disease from the bone marrow by high-resolution flow cytometry.

FIG. 9B shows the peak CD4+/EGFRt+(left) and CD8+/EGFRt+(right) CAR-T cell counts in blood from patients who cleared disease from the bone marrow by high-resolution flow cytometry and did or did not have detectable malignant IGH sequences in marrow.

FIG. 10A shows an estimated probability curve of response and an estimated probability curve of developing Grade 3-5 neurotoxicity constructed based on the number of CD4⁺/EGFRt⁺ or CD8⁺/EGFRt⁺ CAR-T cells in the blood.

FIG. 10B shows estimated probability curves of bone marrow response, complete or partial remission (CR/PR) by IWCLL criteria, developing Grade 2-5 cytokine release syndrome (CRS), and developing Grade 2-5 neurotoxicity (NT) constructed based on the number of CD4⁺/EGFRt⁺ or CD8⁺/EGFRt⁺ CAR-T cells in the blood.

FIG. 11 shows a waterfall plot of the change in cross-sectional area of the 6 largest lymph nodes on CT scan by IWCLL (2008) imaging criteria at best response in high-risk CLL patients after CAR-T cell immunotherapy. Four patients (2 CR, 1 SD, 1 died) without high-resolution imaging to enable tumor measurement are not shown.

### DETAILED DESCRIPTION

### I. Methods and Uses of Cell Therapy with Genetically Engineered Cells

Provided are methods and compositions for use in cell therapy, for the treatment of diseases or conditions, including various cancers and tumors. The methods involve administering engineered cells expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

In some embodiments, the cells, populations, and compositions are administered to a subject having the particular disease or condition to be treated, e.g., via adoptive cell therapy, such as adoptive T cell therapy. In some embodiments, the methods involve treating a subject having a chronic lymphocytic leukemia (CLL) or a non-Hodgkin lymphoma (NHL) with a dose of antigen receptor-expressing cells (e.g. CAR-expressing cells).

In some embodiments, the subject has been preconditioned with an immunodepleting (e.g. lymphodepleting) therapy. Preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., 2002 Science, 298, 850-54; Rosenberg et al., Clin Cancer Res 2011, 17(13):4550-4557. Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. 2006 December; 3(12): 668-681.

Thus, in some embodiments, the methods comprise administration of a chemotherapeutic agent, e.g., a conditioning chemotherapeutic agent, for example, to reduce tumor burden prior to administering the dose of cells. In some embodiments, the methods include administering a preconditioning agent, such as a lymphodepleting or chemotherapeutic agent, such as cyclophosphamide, fludarabine, or combinations thereof. In some embodiments, the methods include administration of fludarabine and, optionally, another chemotherapeutic other than fludarabine. In some embodiments, the other chemotherapeutic agent is cyclophosphamide. In some embodiments, the subject may be administered a lymphodepleting therapy at least 2 days prior, such as at least 3, 4, 5, 6, or 7 days prior, to the administration of the dose of cells. In some embodiments, the lymphodepleting therapy is administered or is initiated at least or at least about or at or about 48 hours or at least or at least about 96 hours prior to the administration of the dose of cells. In some embodiments, the lymphodepleting therapy is administered or is initiated between at or about 48 hours and at or about 96 hours prior to administration of the dose of cells.

Thus, in some embodiments, the methods include administering a preconditioning agent, such as a lymphodepleting or chemotherapeutic agent, such as cyclophosphamide, fludarabine, or combinations thereof, to a subject prior to the first or subsequent dose. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the first or subsequent dose.

In some embodiments, the subject is preconditioned with cyclophosphamide at a dose between or between about 20 mg/kg and 100 mg/kg, such as between or between about 40 mg/kg and 80 mg/kg or between or between about 30 mg/kg and 60 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², inclusive, such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², 20 mg/m² and 30 mg/m², 24 mg/m² and 35 mg/m² or 24 mg/m² and 26 mg/m². In some instances, the subject is administered 25 mg/m² of fludarabine. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 30-60 mg/kg (˜1-2 g/m²) of cyclophosphamide and 3 to 5 doses of 25 mg/m² fludarabine prior to the dose of cells. In some aspects, the subject is administered 60 mg/kg (˜2 g/m²) of cyclophosphamide and 3 to 5 doses of 25 mg/m² fludarabine prior to the first or subsequent dose. In some embodiments, lymphodepletion chemotherapy can be modified by reducing or omitting the dose of cyclophosphamide or administering a regimen of a lower total dose of cyclophosphamide administered concurrently with fludarabine, to minimize toxicity in subjects, such as subjects who have received multiple previous cycles of chemotherapy, have previously undergone allogeneic transplantation, have poor marrow reserve, and/or have other serious comorbidities.

In some embodiments, the antigen receptor (e.g. CAR) specifically binds to a target antigen associated with the disease or condition, such as associated with CLL or NHL. In some embodiments, the antigen associated with the disease or disorder is selected from CD20, CD19, CD22, ROR1, CD45, CD21, CD5, CD33, Igkappa, Iglambda, CD79a, CD79b or CD30.

In some embodiments, the methods include administration of the cells or a composition containing the cells to a subject, tissue, or cell, such as one having, at risk for, or suspected of having the disease, condition or disorder. In some embodiments, the subject is the subject is an adult. In some embodiments, the subject is over at or about 30, 40, 50, 60, or 70 years of age.

In some embodiments, the provided methods involve adoptive cell therapy methods, e.g. CAR+ T cells, for treating chronic lymphocytic leukemia (CLL). CLL is the most common adult leukemia. In some cases, patients with high-risk disease, including those that manifest by del(17)(p13.1), p53 mutation, complex kayotype or umutated immunoglobulin variable regions, require earlier therapy and/or have shorter survival (Dohner et al. (2000) N. Engl. J. Med., 343:1910-1916; Stilgenbauer et al. (2014) Blood, 123:3247-3254; Thompson et al. (2015) Cancer, 121:3612-3621). Among treatments for high-risk CLL include chemotherapy (Hallek et al. (2010) Lancet 376:1164-1174), although recently the BTK inhibitor, ibrutinib, has recently been approved initially for relapsed and refractory disease and subsequently for first-line therapy (Burger et al. (2015) N. Engl J. Med., 373:2425-2437; Byrd et al. (2013) N. Engl. J. Med., 369:32-42). While the overall response rate (ORR) to ibrutinib is high, the complete response rate (CR; or complete remission) can, in some cases, be low, and survival of patients who progress on ibrutinib may be short. Another treatment for high-risk CLL is the BCL2-inhibitor, venetoclax, which as shown activity in some patients who failed ibrutinib therapy, but CR is rare and durability has not been reported (Stilgenbauer et al. (2016) Lancet Oncol., 17:768-778).

T cell-based therapies, such as adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. The engineered expression of recombinant receptors, such as chimeric antigen receptors (CARs), on the surface of T cells enables the redirection of T-cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In some embodiments, the provided methods and uses provide for or achieve improved or more durable responses or efficacy as compared to certain alternative methods, such as in particular groups of subjects treated, such as in patients with a leukemia, such as CLL or NHL, including those with high-risk disease. In some embodiments, the methods are advantageous by virtue of administering T cell therapy, such as a composition including cells for adoptive cell therapy, e.g., such as a T cell therapy (e.g. CAR-expressing T cells), and a lymphodepleting therapy, e.g. such as cyclophosphamide, fludarabine, or combinations thereof. In some embodiments, the provided methods are based on observations that a high rate of elimination of marrow disease and molecular CR can be achieved in patients with high-risk ibrutinib refractory CLL after lympodepletion and CLL-targeted CAR-T cell therapy, such as anti-CD19 CAR+ T cell therapy. This result was achieved with relatively low incidence of serious toxicity that was generally manageable.

In some embodiments, the methods include administration of cells to a subject selected or identified as having a certain prognosis or risk of CLL. Chronic lymphocytic leukemia (CLL) is a generally a variable disease. Some subjects with CLL may survive without treatment while others may require immediate intervention. In some cases, subjects with CLL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the method are classified or identified based on the risk of CLL. In some embodiments, the subject is one that has high risk CLL.

In some cases, one method of classifying subjects is the Rai system. In some aspects, the Rai system comprises 5 stages: Rai stage 0: lymphocytosis and no enlargement of the lymph nodes, spleen, or liver, and with near normal red blood cell and platelet counts; lymphocytes in blood >15000/mcL, and >40% lymphocytes in the bone marrow. Rai stage I: lymphocytosis plus enlarged lymph nodes. The spleen and liver are not enlarged and the red blood cell and platelet counts are near normal. Rai stage II: lymphocytosis plus an enlarged spleen (and possibly an enlarged liver), with or without enlarged lymph nodes. The red blood cell and platelet counts are near normal. Rai stage III: lymphocytosis plus anemia (too few red blood cells), with or without enlarged lymph nodes, spleen, or liver. Platelet counts are near normal. Rai stage IV: lymphocytosis plus thrombocytopenia (too few blood platelets), with or without anemia, enlarged lymph nodes, spleen, or liver. Rai stages may be further grouped into risk groups as follows: Stage 0 is considered low risk; stages I and II are considered intermediate risk; stages III and IV are considered high risk and in some grading systems also include disease-related anemia (hemoglobin level <11.0 g/dL or hematocrit <33%) or platelets <100,000/mcL. In some cases, subjects may also be, or alternatively be, grouped into classes according to the Binet system, which in some respects involves assessing the number of affected lymphoid tissue groups (neck lymph nodes, groin lymph nodes, underarm lymph nodes, spleen, and liver) and by whether or not the patient has anemia (too few red blood cells) or thrombocytopenia (too few blood platelets). In some aspects, the Binet system comprises three stages: Binet stage A: Hemoglobin ≥10 g/dL, platelets ≥100,000/mm3, and <3 enlarged areas; Binet stage B: Hemoglobin ≥10 g/dL, platelets ≥100,000/mm3, and ≥3 enlarged areas; and Binet stage C: Hemoglobin <10 g/dL, platelets <100,000/mm3, and any number of enlarged areas and anemia and/or thrombocytopenia are present. (Rai K R, Keating H J. Chronic lymphocytic leukemia. In: Cancer Medicine. 4th ed. Baltimore, Md.: Williams and Wilkins; 1997. Vol II: 2697-728; Hallek M. et al. Guidelines for the diagnosis and treatment of chronic lymphocytic leukemia: a report from the International Workshop on Chronic Lymphocytic Leukemia updating the National Cancer Institute-Working Group 1996 guidelines. Blood 2008. 111:5446-56.).

Additional methods of classifying subjects with CLL involve genetic analysis and determination of the presence or absence of genetic abnormalities. In some cases, prognostic genetic abnormalities include cytogenetic abnormalities, which may include deletion of the long arm of chromosome 13 (del 13q), del 11q, trisomy 12, del 17p and del 6q. Deletions of 11q, 13q, 17p, and trisomy 12 can have prognostic value and may contribute to CLL pathogenesis and evolution and inform outcome and therapeutic strategies. Abnormalities also include some that are not detectable by more traditional methods such as FISH analysis. In some aspects, chromosomal translocations, including unbalanced translocations, and complex karyotype, can be associated with poor outcomes and/or poor prognosis. Complex karyotypes (CK), generally defined as the presence of three or more chromosomal abnormalities, are detected in nearly 16% of CLL subjects and have been associated with unmutated IgHV status and CD38 expression. CK can be predictive of shortened time to first therapy (TTFT) and overall survival (OS) in CLL subjects treated with salvage therapies, including chlorodeoxyadenosine (cdA). The number of karyotypic abnormalities can be associated with shorter progression-free survival (PFS) and OS following hematopoietic stem cell transplantation (HSCT) following conditioning. Other genetic modifications that may indicate that a subject is high or very high risk include mutations of IgVH, ZAP70, and/or CD38. (Gribben, ASH Education Book; Jan. 1, 2008, vol. 2008 no. 1, 444-449; Puiggros et al., BioMed Research International, Volume 2014 (2014), Article ID 435983). In some cases, high or very high risk patients may exhibit one or more genetic abnormalities.

In some embodiments, the subject exhibits one or more cytogenetic abnormalities, such as one or more of complex karyotype, deletion of the long arm of chromosome 13 (del 13q), del 11, trisomy 12, del 17p, del 6q, and del 13q.14. In some embodiments, any one or more of the cytogenetic abnormalities can be detected by fluorescence in situ hybridization (FISH).

In some embodiments, the subjects with CLL exhibit Richter's syndrome (RS). RS is defined as the transformation of CLL into an aggressive lymphoma, most commonly diffuse large B-cell lymphoma (DLBCL) (see, e.g., Parikh et al. Blood 2014 123:1647-1657).

In some embodiments, the subjects with CLL and/or RS exhibit neurological symptoms or neurological complications, such as symptoms or complications in the central nervous system (CNS). In some embodiments, magnetic resonance imaging (MM) of the CNS (e.g. brain, spine) and/or a lumbar puncture with cerebral spinal fluid (CSF) analysis can be used to evaluate CNS-related symptoms, e.g., presence of monoclonal population of lymphocytes in the CSF (see, e.g., Mozzam et al., J. Neurooncol. 2012 106:185-200; Strati et al., Haematologica 2016 101: 458-465).

In some embodiments, the methods include administration of cells to a subject selected or identified as having high-risk NHL. In some embodiments, the subject exhibits one or more cytogenetic abnormalities, such as associated with high-risk NHL. In some embodiments, the subject is selected or identified based on having a disease or condition characterized or determined to be aggressive NHL, diffuse large B cell lymphoma (DLBCL), primary mediastinal large B cell lymphoma (PMBCL), T cell/histocyte-rich large B cell lymphoma (TCHRBCL), Burkitt lymphoma, mantle cell lymphoma (MCL), and/or follicular lymphoma (FL).

In some embodiments, the subject has been previously treated with a therapy or a therapeutic agent targeting the disease or condition, e.g. CLL or NHL, prior to administration of the cells expressing the recombinant antigen receptor. In some embodiments, the therapeutic agent is a kinase inhibitor, such as an inhibitor of Bruton's tyrosine kinase (Btk), for example, ibrutinib. In some embodiments, the therapeutic agent is an inhibitor of B-cell lymphoma-2 (Bcl-2), for example, venetoclax. In some embodiments, the therapeutic agent is an antibody (e.g. monoclonal antibody) that specifically binds to an antigen expressed by the cells of the CLL or NHL, e.g. an antigen from any one or more of CD20, CD19, CD22, ROR1, CD45, CD21, CD5, CD33, Igkappa, Iglambda, CD79a, CD79b or CD30. In some embodiments, the therapeutic agent is an anti-CD20 antibody, e.g., rituximab. In some embodiments, the therapeutic agent is a depleting chemotherapy that is a combination therapy that includes rituximab, e.g., a combination therapy of fludarabine and rituximab or a combination therapy of anthracycline and rituximab. In some embodiments, the subject has been previously treated with hematopoietic stem cell transplantation (HSCT), e.g., allogenic HSCT or autogenic HSCT. In some embodiments, the subject has been treated or has previously received at least or about at least or about 1, 2, 3, or 4 other therapies for treating the NHL or CLL other than the lymphodepleting therapy and/or the dose of cells expressing the antigen receptor. In some embodiments, the subject has been previously treated with chemotherapy or radiation therapy.

In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation.

In some cases, treatments or therapies (or those of particular categories) may not be recommended for CLL subjects in the low and intermediate risk categories. In some cases, treatment strategies for high risk and very high risk subjects may include fludarabine, cyclophosphamide, and rituximab (FCR), BTK inhibitors (e.g. ibrutinib), and/or allogeneic stem cell transplantation. (Puiggros et al., BioMed Research International, Volume 2014 (2014), Article ID 435983). In some aspects, subjects treated for CLL exhibit poor long-term outcomes. For example, in some cases, refractory (R/R) high-risk CLL subjects exhibit poor survival after Ibrutinib discontinuation (Jain et al. (2015) Blood 125(13):2062-2067). There is a need for improved methods of treating CLL, and in some aspects, for those appropriate for treating high and/or very high-risk CLL and/or subjects having relapsed or become refractory to multiple prior therapies.

In some embodiments, the provided methods are for use in subjects having a cancer in which the subject and/or the cancer is resistant to inhibition by irbrutinib or comprises a population of cells that are resistant to inhibition by the inhibitor. In some aspects, the patient is one that is selected that is or is likely to become refractory to ibrutinib based on high-risk cytogenetics and/or based on the presence of, such as by early detection of, mutations prior to relapse that confer ibrutinib resistance. In some embodiments, provided methods are for use in a subject having a cancer in which the subject and/or the cancer comprises a mutation or disruption in a nucleic acid encoding BTK, in which such mutation is capable of reducing or preventing inhibition of the BTK by the inhibitor, e.g. ibrutinib. In some aspects of any of the methods provided herein, the mutation in the nucleic acid encoding BTK contains a substitution at position C481, optionally C481S or C481R, and/or a substitution at position T474, optionally T474I or T474M. In some embodiments, the provided methods are for use in a subject having a cancer in which at the time of administration of the adoptive cell therapy, e.g., such as a T cell therapy (e.g. CAR-expressing T cells) and/or at the time of administering the lymphodepleting therapy, the subject has relapsed following remission after treatment with, or been deemed refractory to treatment with ibrutinib.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, and following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In certain embodiments, engineered cells are modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

Also provided herein are methods of prognosis or staging of subjects after treatment with cell therapy, e.g., such as a T cell therapy (e.g. CAR-expressing T cells), methods of monitoring response in subjects having received such a cell therapy and/or methods of predicting durability of response to such a cell therapy, in which such methods involve sequencing of the immunoglobulin heavy chain (IGH) locus in samples containing or potentially containing tumor cells, obtained from of subjects that have received the cell therapy. In some embodiments, such methods involve sequencing the IGH locus of harvested samples, such as those potentially leukemia cells, e.g. B cells, from marrow or blood, and/or samples prepared therefrom, to detect the presence or absence of residual tumor.

In some embodiments, typical methods for prognosis, staging and/or monitoring response rates in subjects having a leukemia or lymphoma, e.g. CLL, to a therapy involve measuring lymph node size. For example, a common criteria for assessing response rates is the International Workshop on Chronic Lymphocytic Leukemia (IWCLL) response criteria (Hallek, et al., Blood 2008, Jun. 15; 111(12): 5446-5456), which, in some aspects, requires that all lymph nodes be ≤15 mm. In some cases, however, it is found that responses to cell therapy is rapid and may be achieved prior to measurable change in lymph node size. Thus, in some aspects, early restaging by tumor criteria alone, such as within or about 4 weeks after cell therapy, may not be an optimal determinant of prognosis. Based on observations provided herein, it is found that, even as early as 4 weeks after administration of a cell therapy, e.g. T cell therapy, such as CAR+ T cells, high rates of elimination of CLL from marrow is observed by IGH sequencing. These results indicate that early restaging of subjects by IGH sequencing may provide for an efficient and earlier prognosis or predictor of response and durability of response compared to methods that rely on tumor size criteria. In some embodiments of the provided methods, IGH sequencing is performed no more than 3 months, such as no more than 2 months or no more than 1 month after initiation of administration of a cell therapy. In some embodiments, IGH sequencing is carried out on at or about or within 2 weeks, 3 weeks, 4 weeks, 5 weeks, 6 weeks, 7 weeks, 8 weeks, 9 weeks, 10 weeks, 11 weeks or 12 weeks after initiation of the cell therapy.

In some embodiments, the IGH sequence methods are used to provide prognostic information for stage a subject after treatment with a cell therapy. In some embodiments, if lack of detectable malignant IGH copies are detected or observed, the subject is identified as responding to the cell therapy, likely to be responding, and/or likely to exhibit or develop a response such as a CR and/or a durable response. In some embodiments, lack of detection of malignant IGH copies by the provided methods is used to identify, such as identify early, e.g. within or about within 3-6 weeks of initiation of administration of the cell therapy, such as within or about 4 weeks of the initiation, whether the subject is exhibiting or is likely to exhibit a response to the cell therapy such as exhibiting a complete response (CR) or overall response (OR) to the cell therapy. In some aspects, if malignant IGH copies are detected, the subject is identified as not responding to the cell therapy and/or not exhibiting a complete response and/or to have poorer prognosis or to need additional treatment. In some embodiments, detection of malignant IGH copies by the provided methods is used to identify, such as identify early, e.g. within or about within 3-6 weeks of initiation of administration of the cell therapy, such as or about 4 weeks of the initiation, if the subject is not exhibiting a response to the cell therapy or is not exhibiting a complete response (CR) or overall response (OR) to the cell therapy. In some aspects, the patient may be identified for possible administration of alternative or additional therapeutic strategies to improve response or likelihood of response. In some embodiments, the methods are carried out on subjects receiving a cell therapy, such as containing engineered T cells, e.g. CAR+ T cells, for treating a B cell malignancy, e.g. CLL or NHL. In some embodiments, the methods are carried out on subjects having received a cell therapy for treating CLL.

In some embodiments, the IGH sequencing methods are used to assess or determine the durability of response to a cell therapy, e.g. CAR+ T cell therapy. In some embodiments, if lack of detectable malignant IGH copies are detected the subject is predicted to exhibit or likely to exhibit a durable response to the cell therapy and/or to be at a low or relatively low risk of relapse or to have a high likelihood of exhibiting progression free survival for at least a certain period of time. In some embodiments, lack of detection of malignant IGH copies by the provided methods is used to identify, such as identify early, e.g. within or about 90 days or earlier of initiation of administration of the cell therapy, subjects predicted to be at lower risk of relapse and/or to have increased likelihood of developing progression free survival (PFS) or a durable response, such as for greater than 3 months, greater than 6 months, greater than 9 months or more. In some aspects, if malignant IGH copies are detected, the subject is predicted not to exhibit or not likely to exhibit a durable response to the cell therapy and/or to be at a high or relatively high risk of relapse or to have a low likelihood of exhibiting progression free survival for at least a certain period of time. In some embodiments, detection of malignant IGH copies by the provided methods is used to identify, such as to identify early, e.g. within or about 90 days or earlier of initiation of administration of the cell therapy, subjects predicted to be at a high risk of relapse and/or to have decreased likelihood of developing progression free survival (PFS) or a durable response, such as for less than 3 months, less than 6 months, less than 9 months or less. In some aspects, the patient may be identified for possible administration of alternative or additional therapeutic strategies to improve response efficacy and/or durability. In some embodiments, the methods are carried out on subjects receiving a cell therapy, such as containing engineered T cells, e.g. CAR+ T cells, for treating a B cell malignancy, e.g. CLL or NHL.

A. Dosing

In some embodiments, a dose of cells is administered to subjects in accord with the provided methods. In some embodiments, the size or timing of the doses is determined as a function of the particular disease or condition in the subject. It is within the level of a skilled artisan to empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, where the subject is a human, the dose includes between about 1×10⁶ and 3×10⁸ total recombinant receptor (e.g., CAR)-expressing cells, e.g., in the range of about 1×10⁷ to 2×10⁸ such cells, such as 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁵ to 5×10⁸ total recombinant receptor-expressing T cells or total T cells, 1×10⁵ to 1×10⁸ total recombinant receptor-expressing T cells or total T cells, from or from about 5×10⁵ to 1×10⁷ total recombinant receptor-expressing T cells or total T cells, or from or from about 1×10⁶ to 1×10⁷ total recombinant receptor-expressing T cells or total T cells, each inclusive.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

B. Response, Efficacy and Survival

In some embodiments, the administration effectively treats the subject despite the subject having become resistant to another therapy. In some embodiments, at least or about at least 50% of subjects, at least or about at least 60% of the subjects, at least or about at least 70% of the subjects, at least or about at least 80% of the subjects or at least or about at least 90% of the subjects treated according to the method achieve complete remission (CR) and/or achieve an objective response (OR).

In some aspects, the administration in accord with the provided methods generally reduces or prevents the expansion or burden of the disease or condition in the subject. For example, where the disease or condition is a tumor, the methods generally reduce tumor size, bulk, metastasis, percentage of blasts in the bone marrow or molecularly detectable cancer, and/or improve prognosis or survival or other symptom associated with tumor burden.

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one in which the subject receives one or more alternative therapeutic agents and/or one in which the subject does not receive a dose of cells and/or a lymphodepleting agent in accord with the provided methods. In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, the event-free survival rate or overall survival rate of the subject is improved by the methods, as compared with other methods, for example, methods in which the subject receives one or more alternative therapeutic agents and/or one in which the subject does not receive a dose of cells and/or a lymphodepleting agent in accord with the provided methods. For example, in some embodiments, event-free survival rate or probability for subjects treated by the methods at 6 months following the dose is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods, for example, methods in which the subject receives one or more alternative therapeutic agents and/or one in which the subject does not receive a dose of cells and/or a lymphodepleting agent in accord with the provided methods. For example, in some embodiments, the probability of relapse at 6 months following the first dose is less than about 80%, less than about 70%, less than about 60%, less than about 50%, less than about 40%, less than about 30%, less than about 20%, or less than about 10%.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some aspects, response assessment utilizes any of clinical, hematologic, and/or molecular methods.

1. IWCLL Response Criteria

In some aspects, response rates in subjects, such as subjects with CLL, are based on the International Workshop on Chronic Lymphocytic Leukemia (IWCLL) response criteria (Hallek, et al., Blood 2008, Jun. 15; 111(12): 5446-5456; also called IWCLL 2008). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to >5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

2. Disease in Bone Marrow or Blood

In some embodiments, a subject has leukemia. The extent of disease burden can be determined by assessment of residual leukemia in blood or bone marrow.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 10,000 normal cells or 1 leukemia cell in 100,000 normal cells. In some embodiments, a subject exhibits MRD that is molecularly detectable if at least or greater than 1 leukemia cell in 10,000 cells detected or 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, an index clone of the leukemia, e.g. CLL, is not detected in the bone marrow of the subject (or in the bone marrow of greater than 50%, 60%, 70%, 80%, 90% or more of the subjects treated according to the methods. In some embodiments, an index clone of the leukemia, e.g. CLL, is assessed by IGH deep sequencing. In some embodiments, the index clone is not detected at a time that is at or about or at least at or about 1, 2, 3, 4, 5, 6, 12, 18 or 24 months following the administration of the cells.

a. Determination of MRD by flow cytometry

In some aspects MRD is detected by flow cytometry. Flow cytometry can be used to monitor bone marrow and peripheral blood samples for cancer cells. In particular aspects, flow cytometry is used to detect or monitor the presence of cancer cells in bone marrow. In some aspects, multiparameter immunological detection by flow cytometry is used to detect cancer cells (see for example, Coustan-Smith et al., (1998) Lancet 351:550-554). In some aspects, multiparameter immunological detection by mass cytometry is used to detect cancer cells. In some examples, 1, 2, 3, 4, 5, 6, 7, 8, 9, 10, 11, 12, 13, 14, 15, 20, 25, 30, 35, 40, 45 or 50 parameters can be used to detect cancer cells. The antigens used for detection are selected based on the cancer being detected (Foon and Todd (1986) Blood 68:1-31).

In some examples, bone marrow is harvested by bone marrow aspirates or bone marrow biopsies, and lymphocytes are isolated for analysis. Monoclonal and/or polyclonal antibodies conjugated to a fluorochrome (e.g., fluorescein isothiocyanate (FITC), phycoerythrin, peridinin chlorophyll protein, or biotin) can be used to detect epitopes, such as terminal deoxynucleotidyl transferase (TdT), CD3, CD10, CD11c, CD13, CD14, CD33, CD19, CD20, CD21, CD22, CD23, CD34, CD45, CD56, CD79b, IgM, and/or KORSA3544, on isolated lymphocytes. Labeled cells can then be detected using flow cytometry, such as multiparameter flow cytometry, or mass cytometry, to detect multiple epitopes.

Lymphoid cells can be identified and gated based on a light-scatter dot plot and then secondarily gated to identify cell populations expressing the immunophenotypic features of interest. Exemplary epitopes are set forth in Table 1 below. Other immunologic classification of leukemias and lymphomas are provided by Foon and Todd (Blood (1986) 68(1): 1-31). In some aspects, flow cytometric assessment of MRD can be achieved by quantifying live lymphocytes bearing one or more CLL immunophenotypes (e.g., low forward/side scatter; CD3^{neg}; CD5⁺; CD14^{neg}; CD19⁺; CD23⁺: CD45⁺: CD56^{neg}.

TABLE 1
Exemplary Immnunophenotype and Cytogentics Characteristics

| Disease | Immunophenotype | Cytogenetics |
|---|---|---|
| Chronic | Pan-B+; CD5+; | Trisomy12 |
| Lymphocytic | CD23+; CD79b/CD22 | del(13)(q14.3) |
| Leukemia (CLL) | weak; FMC7−; sIg | del 11q22-q23 |
| | weak | del 17p13 (p53) |
| | | t(11; 14)(q13; q32) BCL1/IgH rearrangement |
| | | t(14; 19)(q32; q13) |
| | | IgH deletion (14q32) |
| | | del(6q) |
| | | +8q24 |
| | | +3 |
| | | +18 |
| | | del 6q21 |
| Small lymphocytic | Pan-B+; CD5+; | del(6)(q21-23) |
| lymphoma (SLL) | CD23+; CD10−; | |
| | sIgM+ faint | |
| Lymphoplasmacytic | Pan-B+; CD5−; CD10−; | t(9; 14)(p13; q32) PAX5/IgH |
| lymphoma | cyIgM+ | |
| Follicle centre cell | Pan-B+; CD10+/−; | t(14; 18)(q32; q21)/BCL2 Rearr |
| lymphoma | CD5−; sIg+ | |
| Diffuse large cell | CD19+; CD22+; | t(14; 18) and p53 mutations |
| lymphoma | CD10−/+; SIg+ | t(3; V)(q27; V)/BCL6 Rearr |
| | | variants c-MYC Rearr |
| Burkitt's lymphoma | Pan-B+; TdT−; | t(8; 14)(q24; q32) or variants/c-MYC R earr |
| | CD10+; CD5−; sIgM+ | |
| Burkitt-like | Pan-B+; TdT−; CD10−/+ | t(8; 14) or variants |
| lymphoma | CD5−; sIg+ | t(8; 14) + t(14; 18) |
| Mantle cell | Pan-B+; CD5+; | t(11; 14)(q13; q32)/BCL1 Rearr |
| lymphoma | CD23−; CD10−/+; | |
| | sIgM+ bright | |
| Marginal zone B-cell | pan-B+; CD5−/+; | t(11; 18)(q21; q21)/PI2/MLT fusion: Extra- |
| lymphoma | CD10−; CD23−; | nodal low-grade MALT lymphoma; |
| (MZBCL) | CD11c+/−; cyIg + | indolent disease |
| | (40% of the cells), | t(1; 14)(p21; q32): Extra-nodal MALT |
| | sIgM+ bright; sIgD− | lymphoma |
| | | del(7)(q22-31): Splenic MZBCL/+3q: |
| | | Nodal, extra-nodal and splenic MZBCL |

+: positive in >90% of the cases
+/−: positive in more than 50% of the cases
−/+: positive in less than 50% of cases
−: positive in <10% of the cases
Pan-B markers: e.g., CD19, CD20, CD79a
sIG: surface immunoglobulins
cyIg: cytoplasmic immunoglobulins

b. IGH Deep Sequencing

In some aspects, deep sequencing of the immunoglobulin heavy chain (IGH) locus of harvested B cells can be used to detect minimal residual disease (MRD). Clonal presence of a particular IgG rearrangement can provide a marker to detect the presence of B cell malignancies, such as CLL or NHL and/or residual presence of malignant cells thereof. In some aspects cells such as a population containing or suspected of containing B cells are harvested and isolated from blood. In some aspects, cells are harvested and isolated from bone marrow, e.g., from bone marrow aspirates or bone marrow biopsies and/or from other biological samples. In some aspects, polymerase chain reaction (PCR) amplification of the complementarity determining region 3 (CDR3) is achieved using primers to highly conserved sequences within the V and J regions of the gene locus, which may be used to identify clonal populations of cells for purposes of assessing minimal residual disease. Other methods for detecting clonal populations, such as single cell sequencing approaches, including those providing information regarding number of cells of a particular lineage and/or expressing a particular variable chain such as variable heavy chain or binding site thereof, such as a clonal population, may be used. In some aspects, the IGH DNA is amplified using a degenerate primers or primers recognizing regions of variable chains shared among different cell clones, such as those recognizing consensus V and degenerate consensus J region of the IGH sequence. An exemplary sequence of the V region is ACACGGCCTCGTGTATTACTGT (SEQ ID NO: 17). An exemplary degenerate consensus sequence of the J region is ACCTGAGGAGACGGTGACC (SEQ ID NO: 18).

The PCR product or sequencing result in some aspects is specific to the rearranged allele and serves as a clonal marker for MRD detection. Following PCR amplification of the CDR3 region, PCR products can be sequenced to yield patient-specific oligonucleotides constructed as probes for allele-specific PCR for sensitive detection of MRD following treatment of B-cell malignancies with CAR-T cell therapy, e.g. CD19 CAR− T cell therapy. In examples where a PCR product is not generated using the consensus primers, V region family-specific primers for the framework region 1 can be used instead.

In some aspects, persistence of PCR-detectable tumor cells such as cells of the B cell malignancy such as the NHL or CLL, such as detectable IGH sequences corresponding to the malignant or clonal IGH sequences, after treatment is associated with increased risk of relapse. In some aspects, patients who are negative for malignant IGH sequences following treatment (in some aspects, even in the context of other criteria indicating progressive disease or only a partial response, such as persistence of enlarged lymph nodes or other criteria that may in some contexts be associated with disease or lack of complete response) may be deemed to have increased likelihood of PFS or to enter into CR or durable CR or prolonged survival, compared to patients with persistent malignant IGH sequences. In some embodiments, such prognostic and staging determinations are particularly relevant for treatments in which clearance of malignant cells is observed within a short period of time following administration of the therapy, e.g., in comparison to resolution of other clinical symptoms such as lymph node size or other staging criteria. For example, in some such aspects, absence of detectable IGH or minimal residual disease in a sample such as the bone marrow may be a preferred readout for response or likelihood of response or durability thereof, as compared to other available staging or prognostic approaches. In some aspects, results from MRD, e.g., IGH deep sequencing information, may inform further intervention or lack thereof. For example, the methods and other provided embodiments in some contexts provide that a subject deemed negative for malignant IGH may in some aspects be not further treated or not be further administered a dose of the therapy provided, or that the subject be administered a lower or reduced dose. Conversely, it may be provided or specified that a subject exhibiting MRD via IGH deep sequencing be further treated, e.g., with the therapy initially administered at a similar or higher dose or with a further treatment.

3. Lugano Criteria

In some respects, response is assessed using the Lugano criteria (Cheson et al., JCO Sep. 20, 2014 vol. 32 no. 27 3059-3067; Johnson et al., (2015) Imaging for staging and response assessment in lymphoma. Radiology 2:323-338). Lugano criteria include evaluation by imaging, tumor bulk measurements, and assessments of spleen, liver, and bone marrow involvement.

In some aspects, response assessed using the Lugano criteria involves the use of positron emission tomography (PET)-computed tomography (CT) and/or CT as appropriate for imaging evaluation. PET-CT evaluations may further comprise the use of fluorodeoxyglucose (FDG), to assess FDG uptake, in FDG-avid lymphomas. FDG-avid lymphomas include Hodgkin lymphoma (HL) and certain non-Hodgkin lymphomas (NHL), including diffuse large B cellular lymphoma (DLBCL), marginal zone NHL with an aggressive transformation, and FDG-avid nodal lymphomas (essentially all histologic types except: chronic lymphocytic leukemia (CLL), small lymphocytic lymphoma, lymphoplasmacytic lymphoma/Waldenström macroglobulinaemia, and mycosis fungoides). In some cases, for non-FDG-avid histologies, CT is the preferred imaging method. In some aspects, the post-treatment scans are taken as long as possible after administration of treatment. In some aspects the post-treatment scans are taken a minimum of 3 weeks after therapy, such as 3 weeks, 4 weeks, 5 weeks, 6 weeks, 7 weeks, 8 weeks, 9 weeks, 10 weeks, 11 weeks, 12, weeks or more after administration of treatment.

In some aspects, where PET-CT will be used to assess response in FDG-avid histologies, a 5-point scale, such as the Deauville five-point scale (Deauville 5ps), may be used for evaluation or staging. The Deauville score is based on visual interpretation of fluorodeoxyglucose (FDG) uptake, visualized by PET/CT scans, of each lesion, compared to two reference organs, the mediastinum (i.e., blood pool) and the liver. One assessment (initial staging) is made prior to treatment and a second round of FDG PET/CT scans is used to evaluate residual masses (in comparison to the FDG update in the reference organs) during and/or after treatment. The scale ranges from 1 to 5, where 1 is best and 5 is the worst. Each FDG-avid (or previously FDG-avid) lesion is rated independently. In some respects, the 5-point scale comprises the following criteria: 1, no uptake above background; 2, uptake ≤mediastinum; 3, uptake >mediastinum but ≤liver; 4, uptake moderately >liver; 5, uptake markedly higher than liver (e.g., maximum standard uptake value (SUV_{MAX}>2× liver; 5a) and/or new lesion (on response evaluation) that is possibly related to lymphoma (5b); X, new areas of uptake unlikely to be related to lymphoma.

A Deauville score of 1 or 2 is considered to represent complete metabolic response (CMR) at interim and end of treatment. A Deauville score of 3 also represents CMR, but interpretation of score 3 depends on the timing of the assessment, the clinical context and the treatment. A Deauville score of 4 or 5 at interim is considered to represent partial metabolic response. However, a Deauville score of 4 or 5 at the end of treatment represents residual metabolic disease, if the uptake has reduced from baseline; no metabolic response (NMR) if there is no change in uptake from baseline; and progressive metabolic disease (PMD) if there in an increase in uptake from baseline and/or there are new lesions. At interim and end of treatment, NMR or PMD indicates treatment failure.

In some aspects, a complete response at the end of treatment, as described using the Lugano criteria, involves a complete metabolic response and a complete radiologic response at various measurable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, non-measured lesions and new lesions are assessed, which in the case of CR should be absent. (Cheson et al., JCO Sep. 20, 2014 vol. 32 no. 27 3059-3067; Johnson et al., (2015) Imaging for staging and response assessment in lymphoma. Radiology 2:323-338).

4. Response Evaluation Criteria in Solid Tumors (RECIST) Criteria

In some aspects, Response Evaluation Criteria in Solid Tumors (RECIST) criteria are used to determine objective tumor response; in some aspects, in solid tumors. (Eisenhauer et al., European Journal of Cancer 45 (2009) 228-247.) In some aspects, the RECIST criteria are used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

C. Toxicity

In some embodiments, the method does not cause or reduces the likelihood of toxicity or toxic outcomes, such as cytokine release syndrome (CRS), severe CRS (sCRS), macrophage activation syndrome, tumor lysis syndrome, fever of at least at or about 38 degrees Celsius for three or more days and a plasma level of CRP of at least at or about 20 mg/dL, neurotoxicity and/or neurotoxicity. In some embodiments, at least 50% of subjects treated according to the method (e.g. at least 60%, at least 70%, at least 80%, at least 90% or more of the subjects treated) do not exhibit a toxic outcome (e.g. CRS or neurotoxicity) or do not exhibit a severe toxic outcome (e.g. severe CRS or severe neurotoxicity). In some embodiments, the subject does not exhibit grade 3 or higher neurotoxicity and/or does not exhibit severe CRS, or does not do so within a certain period of time following the treatment, such as within a week, two weeks, or one month of the administration of the cells.

Administration of adoptive T cell therapy, such as treatment with T cells expressing chimeric antigen receptors, can induce toxic effects or outcomes such as cytokine release syndrome and neurotoxicity. In some examples, such effects or outcomes parallel high levels of circulating cytokines, which may underlie the observed toxicity.

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure.

CRS may be treated using anti-inflammatory therapy such as an anti-IL-6 therapy, e.g., anti-IL-6 antibody, e.g., tocilizumab, or antibiotics. Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-α, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1β, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment. In some aspects, the one or more cytokines or chemokines include IFN-γ, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 2 below.

TABLE 2
Exemplary Grading Criteria for CRS

| Grade | Description of Symptoms |
|---|---|
| 1 | Not life-threatening, require only symptomatic treatment |
| Mild | such as antipyretics and anti-emetics (e.g., fever, nausea, |
| | fatigue, headache, myalgias, malaise) |
| 2 | Require and respond to moderate intervention: |
| Moderate | Oxygen requirement < 40%, or |
| | Hypotension responsive to fluids or low dose of a single |
| | vasopressor, or |
| | Grade 2 organ toxicity (by CTCAE v4.0) |
| 3 | Require and respond to aggressive intervention: |
| Severe | Oxygen requirement ≥ 40%, or |
| | Hypotension requiring high dose of a single vasopressor |
| | (e.g., norepinephrine ≥ 20 μg/kg/min, dopamine ≥ |
| | 10 μg/kg/min, phenylephrine ≥ 200 μg/kg/min, or |
| | epinephrine ≥ 10 μg/kg/min), or |
| | Hypotension requiring multiple vasopressors (e.g., |
| | vasopressin + one of the above agents, or combination |
| | vasopressors equivalent to ≥20 μg/kg/min norepinephrine), |
| | or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

As used herein, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 2.

In some embodiments, the CRS encompasses a combination of (1) persistent fever (fever of at least 38 degrees Celsius for at least three days) and (2) a serum level of CRP of at least at or about 20 mg/dL.

In some embodiments, the CRS encompasses hypotension requiring the use of two or more vasopressors or respiratory failure requiring mechanical ventilation.

The method of measuring or detecting the various outcomes may be specified.

In some aspects, the toxic outcome is or is associated with neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

As used herein, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 3.

TABLE 3
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental activities |
| Moderate | of daily living (ADL), such as preparing meals, shopping |
| | for groceries or clothes, using the telephone, |
| | managing money |
| 3 | Presence of symptoms that limit self-care ADL, such as |
| Severe | bathing, dressing and undressing, feeding self, using the |
| | toilet, taking medications |
| 4 | Symptoms that are life-threatening, requiring urgent |
| Life- | intervention |
| threatening | |
| 5 | Death |
| Fatal | |

In some embodiments, the methods reduce symptoms associated with neurotoxicity compared to other methods. For example, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

### II. Recombinant Antigen Receptors Expressed by the Cells

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8+ or CD4+ cells are enriched or selected. Among the compositions are pharmaceutical compositions and formulations for administration, such as for adoptive cell therapy. Also provided are therapeutic methods for administering the cells and compositions to subjects, e.g., patients, in accord with the provided methods.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

A. Chimeric Antigen Receptors (CARs)

In some embodiments of the provided methods and uses, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments the scFv and/or VH domains is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1 and H2 set forth in SEQ ID NOS: 38, 39 respectively, and CDRH3 seq forth in SEQ ID NOS: 40 or 54 and CDRL1 set forth in SEQ ID NOS: 35 and CDR L2 36 or 55 and CDR L3 sequences 37 or 56. The FMC63 antibody comprises the heavy chain variable region (V_{H}) comprising the amino acid sequence of SEQ ID NO: 41 and the light chain variable region (V_{L}) comprising the amino acid sequence of SEQ ID NO: 42. In some embodiments, the svFv comprises a variable light chain containing the CDRL1 sequence of 35, a CDRL2 sequence of 36, and a CDRL3 sequence of 37 and/or a variable heavy chain containing a CDRH1 sequence of 38, a CDRH2 sequence of 39, and a CDRH3 sequence of 40. In some embodiments, the scFv comprises a variable heavy chain region of FMC63 set forth in SEQ ID NO:41 and a variable light chain region of FMC63 set forth in 42. In some embodiments, the variable heavy and variable light chain are connected by a linker. In some embodiments, the linker is set forth in SEQ ID NO:24. In some embodiments, the scFv comprises, in order, a VH, a linker, and a VL. In some embodiments, the scFv comprises, in order, a VL, a linker, and a VH. In some embodiments, the svFc is encoded by a sequence of nucleotides set forth in SEQ ID NO:25 or a sequence that exhibits at least 85%, 86%, 87%, 88%, 89%, 90%, 91%, 92%, 93%, 94%, 95%, 96%, 97%, 98%, or 99% sequence identity to SEQ ID NO:25. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOS: 47-49, respectively, and CDRL1, L2 and L3 sequences set forth in SEQ ID NOS: 44-46, respectively. The SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the svFv comprises a variable light chain containing the CDRL1 sequence of 44, a CDRL2 sequence of 45, and a CDRL3 sequence of 46 and/or a variable heavy chain containing a CDRH1 sequence of 47, a CDRH2 sequence of 48, and a CDRH3 sequence of 49. In some embodiments, the scFv comprises a variable heavy chain region of SJ25C1 set forth in SEQ ID NO:50 and a variable light chain region of SJ25C1 set forth in 51. In some embodiments, the variable heavy and variable light chain are connected by a linker. In some embodiments, the linker is set forth in SEQ ID NO:52. In some embodiments, the scFv comprises, in order, a VH, a linker, and a VL. In some embodiments, the scFv comprises, in order, a VL, a linker, and a VH. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the constant region or portion is of IgD. In some embodiments, the spacer has the sequence set forth in SEQ ID NO: 5. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4 or 5. In some embodiments, the spacer has the sequence set forth in SEQ ID NOS: 26-34. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 26-34.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD 134, CD137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A, P2A, E2A or F2A, e.g. set forth in any of SEQ ID NOS: 6 or 19-23. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO: 1. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes a tEGFR sequence set forth in SEQ ID NO: 7 or 16, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

B. TCRs

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683.

### III. Genetically Engineered Cells and Methods of Producing Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

A. Vectors and Methods for Genetic Engineering

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/WIC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

B. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### IV. Compositions and Formulations

In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the agent or host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### V. Articles of Manufacture and Kits

Also provided are articles of manufacture and kits containing engineered cells expressing a recombinant receptor or compositions thereof, and optionally instructions for use, for example, instructions for administering, according to the provided methods.

In some embodiments, provided are articles of manufacture and/or kits that include a composition comprising a therapeutically effective amount of any of the engineered cells described herein, and instructions for administering, to a subject for treating a disease or condition. In some embodiments, the instructions can specify some or all of the elements of the methods provided herein. In some embodiments, the instructions specify particular instructions for administration of the cells for cell therapy, e.g., doses, timing, selection and/or identification of subjects for administration and conditions for administration. In some embodiments, the articles of manufacture and/or kits further comprise an agent for lymphodepleting therapy, and optionally further includes instructions for administering the lymphodepleting therapy. In some embodiments, the instructions can be included as a label or package insert accompanying the compositions for administration.

In some embodiments, the instructions specify the criteria for selection or identification of subjects for therapy. In some embodiments, such criteria include subjects having a high-risk CLL. In some embodiments, the instructions specify the subject is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH; the subject is or has been identified as having high-risk CLL; and/or the subject is or has been identified as having extramedullary disease; and/or the subject is an adult and/or is over at or about 30, 40, 50, 60, or 70 years of age. In some embodiments, the criteria for selection or identification include that the subjects have a high risk NHL. In some embodiments, the subject is or has been identified as having one or more cytogenetic abnormalities, optionally associated with high-risk NHL; the subject is or has been identified as having high-risk NHL; and/or the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or the subject is an adult and/or is over at or about 30, 40, 50, 60, or 70 years of age.

In some embodiments, the instructions specify that the subject is one that has received one or more previous or prior therapies for treating the leukemia, e.g. CLL or NHL, such as has received 2, 3, or 4 more prior therapies. In some embodiments, the instructions specify the subject is selected for treatment with the cell therapy, e.g. CAR+ T cells, if the subject is identified to be or likely to be refractory to and/or has relapsed after receiving one or more of such prior therapies.

In some embodiments, the instructions specify the dose of cells to be administered. For example, in some embodiments, the instructions specify the dose (i) comprises (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg. In some embodiments, the instructions specify particulars of the cell therapy, including the target of the cell therapy, e.g. that the cell therapy is a CD19-targeted cell therapy. In some embodiments, the instructions specify that the cell therapy includes administration of CAR+ engineered cells that comprise a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1.

In some embodiments, the articles of manufacture and/or kits further include one or more additional agents for therapy, e.g., lymphodepleting therapy and/or combination therapy, as described herein, and optionally instructions for administering the additional agents. In some embodiments, the articles of manufacture and/or kits further include one or more reagents for assaying biological samples, e.g., biological samples from subjects who are candidates for administration or who have been administered the therapy, and optionally instructions for use of the reagents or assays. In some embodiments, the reagents can be used prior to the administration of the cell therapy or after the administration of cell therapy, for diagnostic purposes, to identify subjects and/or to assess treatment outcomes and/or toxicities. For example, in some embodiments, the article of manufacture and/or kits further contain reagents for measuring the level of particular biomarkers, e.g., cytokines, that are associated with toxicity, and instructions for measuring. In some embodiments, the reagents include components for performing an in vitro assay to measure the biomarkers, such as an immunoassay, an aptamer-based assay, a histological or cytological assay, or an mRNA expression level assay. In some embodiments, the in vitro assay is selected from among an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay and avidity assay. In some aspects, the reagent is a binding reagent that specifically binds the biomarkers. In some cases, the binding reagent is an antibody or antigen-binding fragment thereof, an aptamer or a nucleic acid probe.

The articles of manufacture and/or kits may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition, such as a CLL or NHL.

The article of manufacture may include a container with a composition contained therein, wherein the composition includes engineered cells expressing a recombinant receptor; and, in some cases, one or more further containers with a composition contained therein, wherein the composition includes one or more further agent or agents. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. Exemplary Embodiments

Among the provided embodiments are:

1. A method of treating a subject having or suspected of having a chronic lymphocytic leukemia (CLL), the method comprising administering to the subject a dose of cells expressing a chimeric antigen receptor (CAR) that specifically binds to a target antigen expressed by the CLL, said dose comprising (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg,

wherein, prior to the administration, the subject has been preconditioned with a lymphodepleting therapy comprising the administration of fludarabine.

2. A method of treating a subject having or suspected of having a chronic lymphocytic leukemia (CLL), the method comprising administering to the subject a dose of cells expressing a chimeric antigen receptor (CAR) that specifically binds to a target antigen expressed by the CLL, said dose comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells,

wherein, prior to the administration, the subject has been preconditioned with a lymphodepleting therapy comprising the administration of fludarabine.

3. The method of embodiment 1, wherein, at or prior to the administration of the dose of cells:

the subject is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH;

the subject is or has been identified as having high-risk CLL; and/or

the subject is or has been identified as having extramedullary disease; and/or

the subject is or has been identified as having central nervous system (CNS) disease; and/or

the subject is an adult and/or is over at or about 30, 40, 50, 60, or 70 years of age.

4. The method of embodiment 1 or embodiment 3, wherein, prior to the administration of the dose of cells, the subject has been treated with two or more, optionally 3, 4, 5, 6, 7, 8, or 9 or more, therapies for the CLL, other than the lymphodepleting therapy and/or other than another dose of cells expressing the CAR.

5. The method of embodiment 1 or embodiment 3, wherein, prior to the administration of the dose of cells, the subject has been treated with two or more, optionally 3, 4, 5, 6, 7, 8, or 9 or more, therapies for the CLL, other than another dose of cells expressing the CAR or other than another dose of cells expressing the CAR and the preconditioning therapy.

6. The method of any of embodiments 1-5, wherein, prior to the administration of the dose of cells, the subject has been treated for the CLL with a kinase inhibitor, optionally an inhibitor of Btk, optionally ibrutinib.

7. The method of any of embodiments 1-6, wherein, prior to the administration of the dose of cells, the subject has been treated for the CLL with a monoclonal antibody that specifically binds to an antigen expressed by, or previously expressed by, cells of the CLL.

8. The method of any of embodiments 1-7, wherein, prior to the administration of the dose of cells, the subject has been treated for the CLL with venetoclax, a combination therapy comprising fludarabine and rituximab, radiation therapy and/or hematopoietic stem cell transplantation (HSCT).

9. The method of any of embodiments 1-8, wherein, at or immediately prior to the time of the administration of the dose of cells, the subject has relapsed following remission after treatment with, or become refractory to, one or more prior therapies for the CLL.

10. The method of any of embodiments 1-9, further comprising, prior to the administration of the cell dose, administering the lymphodepleting therapy to the subject.

11. The method of any of embodiments 1-10, wherein the lymphodepleting therapy:

(i) further comprises administering another chemotherapeutic agent other than the fludarabine, which optionally is cyclophosphamide;

(ii) is initiated at a time that is at least at or about 48 hours prior to or is between at or about 48 and at or about 96 hours prior to the administration of the cells; and

(iii) comprises the administration of cyclophosphamide at about 30-60 mg/kg, optionally once daily for one or two days, and/or the fludarabine at about 25 mg/m², daily for 3-5 days.

12. The method of any of embodiments 1-11, wherein the administration of the cell dose and/or the lymphodepleting therapy is carried out via outpatient delivery.

13. The method of any of embodiments 1-12, wherein the dose of cells comprises a defined ratio of CD4+ cells expressing the CAR to CD8+ cells expressing the CAR and/or of CD4+ cells to CD8+ cells, which optionally is approximately 1:1 or is between approximately 1:3 and approximately 3:1.

14. The method of any of embodiments 1-13, wherein the dose of cells is administered parenterally, optionally intravenously.

15. The method of any of embodiments 1-14, wherein:

at least 50% of subjects treated according to the method achieve complete remission (CR) and/or objective response (OR); and/or

the subject exhibits CR, OR, or lymph nodes of less than at or about 20 mm in size, within 1 month of the administration of the dose of cells; and/or

wherein a malignant immunoglobulin heavy chain (IGH) locus and/or an index clone of the CLL is not detected in the bone marrow of the subject (or in the bone marrow of greater than 50% of subjects treated according to the methods), optionally as assessed by IGH deep sequencing, optionally at a time that is at or about or at least at or about 1, 2, 3, 4, 5, 6, 12, 18, or 24 months following the administration of the cell dose.

16. The method of any of embodiments 1-15, wherein:

at least 50% of subjects treated according to the method achieve complete remission (CR), exhibit progression-free survival (PFS) and/or overall survival (OS) of greater than 12 months;

on average, subjects treated according to the method exhibit a median PFS or OS of greater than at or about 6 months, 12 months, or 18 months; and/or

the subject exhibits PFS or OS following therapy for at least at or about 6, 12, 18 or more months.

17. The method of any of embodiments 1-16, wherein the antigen is a B cell antigen, which optionally is CD19.

18. The method of any of embodiments 1-17, wherein the CAR comprises an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta.

19. The method of any of embodiments 1-18, wherein the CAR comprises a spacer and/or hinge region, each optionally derived from a human IgG.

20. A method of treating a subject having a non-Hodgkin lymphoma (NHL), the method comprising administering to the subject a dose of cells expressing a chimeric antigen receptor (CAR) that specifically binds to a target antigen expressed by the NHL, wherein:

said dose (i) comprises (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg, and (ii) comprises a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1,

wherein, prior to the administration, the subject has been preconditioned with a lymphodepleting therapy comprising the administration of fludarabine.

21. A method of treating a subject having a non-Hodgkin lymphoma (NHL), the method comprising administering to the subject a dose of cells expressing a chimeric antigen receptor (CAR) that specifically binds to a target antigen expressed by the NHL, wherein:

said dose (i) comprises (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, and (ii) comprises a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1,

wherein, prior to the administration, the subject has been preconditioned with a lymphodepleting therapy comprising the administration of fludarabine.

22. The method of embodiment 20, wherein, at or prior to the administration of the dose of cells:

the subject is or has been identified as having one or more cytogenetic abnormalities, optionally associated with high-risk NHL;

the subject is or has been identified as having high-risk NHL; and/or

the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or

the subject is an adult and/or is over at or about 30, 40, 50, 60, or 70 years of age.

23. The method of embodiment 20 or embodiment 22, wherein, prior to the administration of the dose of cells, the subject has been treated with two or more, optionally 2, 3, or 4 or more, therapies for the NHL other than the lymphodepleting therapy and/or other than another dose of cells expressing the CAR.

24. The method of any of embodiments 20-23, wherein, at or immediately prior to the time of the administration of the dose of cells, the subject has relapsed following remission after treatment with, or become refractory to, one or more prior therapies for the NHL.

25. The method of any of embodiments 20-24, further comprising, prior to the administration of the cell dose, administering the lymphodepleting therapy to the subject.

26. The method of any of embodiments 20-25, wherein the lymphodepleting therapy:

(i) further comprises administering another chemotherapeutic agent other than the fludarabine, which optionally is cyclophosphamide;

(ii) is initiated at a time that is at least at or about 48 hours prior to or is between at or about 48 and at or about 96 hours prior to the administration of the cells; and

(iii) comprises the administration of cyclophosphamide at about 30-60 mg/kg, optionally once daily for one or two days, and/or the fludarabine at about 25 mg/m², daily for 3-5 days.

27. The method of any of embodiments 20-26, wherein the administration of the cell dose and/or the lymphodepleting therapy is carried out via outpatient delivery.

28. The method of any of embodiments 20-27, wherein the defined ratio is a defined ratio of CD4+ cells expressing the CAR to CD8+ cells expressing the CAR of at or about 1:1 and/or is a defined ratio of CD4+ cells to CD8+ cells, which is at or about 1:1.

29. The method of any of embodiments 20-28, wherein the dose of cells is administered parenterally, optionally intravenously.

30. The method of any of embodiments 20-29, wherein at least 50% of subjects treated according to the method achieve complete remission (CR) and/or objective response (OR).

31. The method of any of embodiments 20-30, wherein:

at least 50% of subjects that are treated according to the method, and that achieve complete remission (CR), exhibit progression-free survival (PFS) and/or overall survival (OS) of greater than 12 months;

on average, subjects treated according to the method exhibit a median PFS or OS of greater than at or about 6 months, 12 months, or 18 months; and/or

the subject exhibits PFS or OS following therapy for at least at or about 6, 12, 18 or more months.

32. The method of any of embodiments 20-31, wherein the antigen is a B cell antigen, which optionally is CD19.

33. The method of any of embodiments 20-32, wherein the CAR comprises an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta.

34. The method of any of embodiments 20-33, wherein the CAR comprises a spacer and/or hinge region, each optionally derived from a human IgG.

35. The method of any of embodiments 1-34, wherein:

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain;

and wherein:
the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P, where X1 is glycine, cysteine or arginine and X2 is cysteine or threonine; and/or
the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or
the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or
the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 24.

36. A method of prognosis, the method comprising detecting the presence or absence of a malignant immunoglobulin heavy chain (IGH) locus sequence in a sample from a subject having a B cell malignancy, said subject having previously received administration of a cell therapy comprising a dose or composition of genetically engineered cells expressing a recombinant receptor for treating the B cell malignancy, wherein detecting the presence or absence of the malignant IGH sequence determines the prognosis of the subject in response to the cell therapy.

37. The method of embodiment 36, wherein the detecting the presence or absence of the malignant IGH sequence is carried out within or within about or about 3 to 6 weeks after initiation of the cell therapy, optionally within or within about 4 weeks of initiation of administration of the cell therapy.

38. The method of embodiment 36 or embodiment 37, wherein if the malignant IGH sequence is detected, the subject is identified as not responding or not exhibiting a complete response (CR) or an overall response (OR) to the cell therapy or as likely to relapse to the cell therapy.

39. The method of any of embodiments 36-38, wherein if the malignant IGH sequence is detected identifying the subject as a candidate for further treatment and/or for receiving an altered or alternative treatment.

40. The method of any of embodiments 36-38, wherein if the malignant IGH sequence is detected discontinuing administration of the cell therapy, administering to the subject a further dose of the cell therapy, administering to the subject a higher dose of the cell therapy, administering o the subject a different cell therapy, optionally a cell therapy expressing a different recombinant receptor, and/or administering to the subject an alternative therapeutic agent for treating the B cell malignancy.

41. The method of embodiment 36 or embodiment 37, wherein if the malignant IGH sequence is not detected, the subject is identified as responding to the cell therapy and/or as exhibiting a complete response (CR) or overall response (OR) to the cell therapy or as likely not to relapse to the cell therapy.

42. The method of any of embodiments 36, 37 and 41 wherein if the malignant IGH sequence is not detected, the subject is identified as a candidate for no further treatment and/or is not further treated, optionally is not further treated with the cell therapy and/or is not further treated with an alternative therapy for the B cell malignancy.

43. A method of predicting durability of response to a cell therapy, the method comprising detecting the presence or absence of a malignant immunoglobulin heavy chain locus (IGH) sequence in a sample from a subject having a B cell malignancy, said subject having previously received administration of a cell therapy comprising a dose or composition of genetically engineered cells expressing a recombinant receptor for treating the B cell malignancy, wherein the presence or absence of the malignant IGH sequence predicts the durability of response to the cell therapy.

44. The method of embodiment 43, wherein the detecting the presence or absence of the malignant IGH sequence is carried out within or within about or about 4 weeks, 6 weeks, 8 weeks, 12 weeks or 16 weeks after initiation of the cell therapy.

45. The method of embodiment 43 or embodiment 44, wherein if the malignant IGH sequence is not detected, the subject is predicted to exhibit or likely to exhibit a durable response to the cell therapy and/or to be at a low or relatively low risk of relapse within a certain period of time and/or to have a high likelihood of exhibiting progression free survival for at least a certain period of time.

46. The method of any of embodiments 43-45, wherein if the malignant IGH sequence is not detected, the subject is predicted:

to exhibit survival without progression for greater than or about 3 months, greater than about 6 months, greater than about 9 months or greater than about 12 months after initiation of the cell therapy; and/or

to remain surviving for greater than or greater than about 3 months, greater than or greater than about 6 months, greater than or greater than about 9 months or greater than about 12 months after initiation of the cell therapy; and/or

to exhibit durable CR or OR for greater than or greater than about 3 months, greater than or greater than about 6 months or greater than or greater than about 9 months after initiation of the cell therapy; and/or

not likely to relapse following initiation of administration of the cell therapy, optionally not likely to relapse within 3 months, 6 months or 9 months after initiation of administration of the cell therapy.

47. The method of embodiment 43 or embodiment 44, wherein if the malignant IGH sequence is detected, the subject is predicted to exhibit or likely to exhibit a response to the cell therapy that is not durable and/or to be at a high or relatively high risk of relapse within a certain period of time and/or to have a low likelihood of exhibiting progression free survival for at least a certain period of time.

48. The method of embodiment 43 or embodiment 44, wherein if the malignant IGH sequence is not detected, the subject is s predicted:

not to exhibit survival without progression for greater than or about 3 months, greater than about 6 months, greater than about 9 months or greater than about 12 months after initiation of the cell therapy; and/or

not to remain surviving for greater than or greater than about 3 months, greater than or greater than about 6 months, greater than or greater than about 9 months or greater than about 12 months after initiation of the cell therapy; and/or

not to exhibit durable CR or OR for greater than or greater than about 3 months, greater than or greater than about 6 months or greater than or greater than about 9 months after initiation of the cell therapy.

49. The method of embodiment 43, 44 and 48, wherein if the malignant IGH sequence is detected administering to the subject a further dose of the cell therapy, administering to the subject a higher dose of the cell therapy, administering o the subject a different cell therapy, optionally a cell therapy expressing a different recombinant receptor, and/or administering to the subject an alternative therapeutic agent for treating the B cell malignancy.

50. The method of any of embodiments 36-49, wherein the presence or absence of the malignant IGH sequence is determined by IGH sequencing, optionally comprising PCR amplification of IGH target DNA.

51. The method of any of embodiments 36-38, wherein the sample comprises B cells.

52. The method of any of embodiments 36-51, wherein the sample comprises a blood or bone marrow sample.

53. The method of any of embodiments 36-52, wherein the sample has been obtained from the subject.

54. The method of any of embodiments 36-53, wherein the method is carried out ex vivo.

55. The method of any of embodiments 36-54, wherein the B cell malignancy is a cancer.

56. The method of any of embodiments 36-55, wherein the B cell malignancy is or comprises a leukemia.

57. The method of any of embodiment 37-56, wherein the B cell malignancy comprises an antigen or is associated with an antigen selected from CD19, CD20, CD22, CD30, CD33 or CD38, ROR1.

58. The method of any of embodiments 36-57, wherein the B cell malignancy is selected from and/or is acute lymphoblastic leukemia (ALL), adult ALL, chronic lymphoblastic leukemia (CLL), non-Hodgkin lymphoma (NHL), and Diffuse Large B-Cell Lymphoma (DLBCL).

59. The method of any of embodiments 37-58, wherein the B cell malignancy is or comprises chronic lymphoblastic leukemia (CLL) or high-risk CLL.

60. The method of any of embodiments 37-58, wherein the B cell malignancy is or comprises non-Hodgkin lymphoma (NHL).

61. The method of embodiment 60, wherein the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

62. The method of any of embodiments 37-61, wherein the recombinant receptor specifically binds to an antigen associated with the disease or condition or expressed in cells of the environment of a lesion associated with the B cell malignancy.

63. The method of any of embodiments 37-62, wherein the recombinant receptor is a T cell receptor or a functional non-T cell receptor.

64. The method of any of embodiments 37-63, wherein the recombinant receptor is a chimeric antigen receptor (CAR).

65. The method of embodiment 64, wherein the CAR comprises an extracellular antigen-recognition domain that specifically binds to the antigen and an intracellular signaling domain comprising an ITAM, wherein optionally, the intracellular signaling domain comprises an intracellular domain of a CD3-zeta (CD3) chain; and/or wherein the CAR further comprises a costimulatory signaling region, which optionally comprises a signaling domain of CD28 or 4-1BB.

66. The method of any of embodiments 37-65, wherein the CAR comprises an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta.

67. The method of any of embodiments 37-66, wherein the CAR comprises a spacer and/or hinge region, each optionally derived from a human IgG.

68. The method of any of embodiments 37-67, wherein:

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain; and wherein:

the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P, where X1 is glycine, cysteine or arginine and X2 is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 24.

69. The method of any of embodiments 37-68, wherein the engineered cells comprise T cells, optionally CD4+ and/or CD8+.

70. The method of embodiment 69, wherein the T cells are primary T cells obtained from a subject.

71. The method of any of any of embodiments 37-70, wherein the engineered cells are autologous to the subject.

72. The method of any of embodiments 37-71, wherein the engineered cells are allogeneic to the subject.

73. An article of manufacture comprising one or more dose of a cell therapy, each dose comprising cells expressing a chimeric antigen receptor (CAR), and instructions for administering the cell therapy, wherein:

the instructions specify the dose of cells is to be administered to a subject having a chronic lymphocytic leukemia (CLL); and

the instructions specify administration of a number of CAR-expressing or a number of cells, or specify administration of an amount or volume of one or more formulations corresponding to or containing said specified number of cells, wherein the specified number of cells to be administered comprises a number to administer a dose of cells comprising (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg.

74. An article of manufacture comprising one or more dose of a cell therapy, each dose comprising cells expressing a chimeric antigen receptor (CAR), and instructions for administering the cell therapy, wherein:

the instructions specify the dose of cells is to be administered to a subject having a chronic lymphocytic leukemia (CLL); and

the instructions specify administration of a number of CAR-expressing or a number of cells, or specify administration of an amount or volume of one or more formulations corresponding to or containing said specified number of cells, wherein the specified number of cells to be administered comprises a number to administer a dose of cells comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.

75. The article of manufacture of embodiment 73, further comprising instructions for use with, after or in connection with a lymphodepleting therapy, the lympodepleting therapy comprising fludarabine.

76. The article of manufacture of embodiment 73 or embodiment 75, wherein the instructions specify that the cell therapy is to be administer to a subject that:

is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH;

is or has been identified as having high-risk CLL; and/or

is or has been identified as having extramedullary disease; and/or

is or has been identified as having central nervous system (CNS) disease; and/or

is an adult and/or is over at or about 30, 40, 50, 60, or 70 years of age.

77. The article of manufacture of any of embodiments 73-76, wherein the instructions specify that the cell therapy is to be administered to a subject that:

has been treated with two or more, optionally 3, 4, 5, 6, 7, 8, or 9 or more, therapies for the CLL, other than the lymphodepleting therapy and/or other than another dose of cells expressing the CAR; and/or

has been treated for the CLL with a kinase inhibitor, optionally an inhibitor of Btk, optionally ibrutinib; and/or

has been treated for the CLL with a monoclonal antibody that specifically binds to an antigen expressed by, or previously expressed by, cells of the CLL; and/or

has been treated for the CLL with venetoclax, a combination therapy comprising fludarabine and rituximab, radiation therapy and/or hematopoietic stem cell transplantation (HSCT).

78. The article of manufacture of any of embodiments 73-77, wherein the instructions specify that the cell therapy is to be administered to a subject that has relapsed following remission after treatment with, or become refractory to, one or more prior therapies for the CLL.

79. An article of manufacture comprising one or more dose of a cell therapy, each dose comprising cells expressing a chimeric antigen receptor (CAR), and instructions for administering the cell therapy, wherein:

the instructions specify the dose of cells is to be administered to a subject having a non-Hodgkin lymphoma (NHL); and

the instructions specify administration of a number of CAR-expressing or a number of cells, or specify administration of an amount or volume of one or more formulations corresponding to or containing said specified number of cells, wherein the specified number of cells to be administered comprises a number to administer a dose of cells comprising (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg.

80. An article of manufacture comprising one or more dose of a cell therapy, each dose comprising cells expressing a chimeric antigen receptor (CAR), and instructions for administering the cell therapy, wherein:

the instructions specify the dose of cells is to be administered to a subject having a non-Hodgkin lymphoma (NHL); and

the instructions specify administration of a number of CAR-expressing or a number of cells, or specify administration of an amount or volume of one or more formulations corresponding to or containing said specified number of cells, wherein the specified number of cells to be administered comprises a number to administer a dose of cells comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.

81. The article of manufacture of embodiment 79, further comprising instructions for use with, after or in connection with a lymphodepleting therapy, the lympodepleting therapy comprising fludarabine.

82. The article of manufacture of embodiment 79 or embodiment 81, wherein the instructions specify that the cell therapy is to be administer to a subject that:

is or has been identified as having one or more cytogenetic abnormalities, optionally associated with high-risk NHL;

is or has been identified as having high-risk NHL; and/or

is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or

is an adult and/or is over at or about 30, 40, 50, 60, or 70 years of age. 83. The article of manufacture of any of embodiments 79-82, wherein the instructions specify that the cell therapy is to be administered to a subject that has been treated with two or more, optionally 2, 3 or 4 or more, therapies for the NHL, other than the lymphodepleting therapy and/or other than another dose of cells expressing the CAR.

84. The article of manufacture of any of embodiments 79-83, wherein the instructions specify that the cell therapy is to be administered to a subject that has relapsed following remission after treatment with, or become refractory to, one or more prior therapies for the NHL.

85. The article of manufacture of any of embodiments 73-84, wherein the lymphodepleting therapy:
(i) further comprises administering another chemotherapeutic agent other than the fludarabine, which optionally is cyclophosphamide; and/or
(ii) comprises the administration of cyclophosphamide at about 30-60 mg/kg, optionally once daily for one or two days, and/or the fludarabine at about 25 mg/m², daily for 3-5 days.

86. The article of manufacture of any of embodiments 73-84, wherein the instructions specify that the lympodepleting therapy is initiated at a time that is at least at or about 48 hours prior to or is between at or about 48 and at or about 96 hours prior to the administration of the cell therapy.

87. The article of manufacture of any of embodiments 73-86, wherein the instructions specify administering the cell therapy at a defined ratio of CD4⁺ cells expressing the CAR to CD8⁺ cells, or specify administering amounts of volumes of the formulation(s) corresponding to such defined ratio, or comprises a formulation having the cells at such ratio or comprises the cells at such ratio expressing the CAR and/or of CD4⁺ cells to CD8⁺ cells, which ratio optionally is approximately 1:1 or is between approximately 1:3 and approximately 3:1.

88. The article of manufacture of any of embodiments 73-87, wherein the instructions further specify the cell therapy is for parenteral administration, optionally intravenous administration.

89. The article of manufacture of any of embodiments 73-88, wherein the instructions further specify the administration of the cell therapy is to be or may be administered to the subject on an outpatient setting and/or without admission of the subject to the hospital overnight or for one or more consecutive days and/or is without admission of the subject to the hospital for one or more days.

90. The article of manufacture of any of embodiments 73-89, wherein the cell therapy comprises primary T cells obtained from a subject.

91. The article of manufacture of embodiment 89, wherein the T cells are autologous to the subject.

92. The article of manufacture of embodiment 91, wherein the T cells are allogeneic to the subject.

93. The article of manufacture of any of embodiments 73-92, wherein the CAR comprises an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta.

94. The article of manufacture of any of embodiments 73-93, wherein the CAR comprises a spacer and/or hinge region, each optionally derived from a human IgG.

95. The article of manufacture of embodiment 93 or embodiment 94, wherein the antigen is a B cell antigen, which optionally is CD19.

96. The article of manufacture of any of embodiments 73-95, wherein:

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain; and wherein:

the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P, where X1 is glycine, cysteine or arginine and X2 is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 24.

97. A composition comprising cells expressing a chimeric antigen receptor (CAR) that specifically binds to a target antigen of a chronic lymphocytic leukemia (CLL) for use in treating a subject having or suspected of having CLL, wherein the treating comprises administering to the subject a dose of cells expressing the CAR, said dose comprising (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg,

wherein, prior to the administration, the subject has been preconditioned with a lymphodepleting therapy comprising the administration of fludarabine.

98. A composition comprising cells expressing a chimeric antigen receptor (CAR) that specifically binds to a target antigen of a chronic lymphocytic leukemia (CLL) for use in treating a subject having or suspected of having CLL, wherein the treating comprises administering to the subject a dose of cells expressing the CAR, said dose comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells,

wherein, prior to the administration, the subject has been preconditioned with a lymphodepleting therapy comprising the administration of fludarabine.

99. The use of embodiment 97 or embodiment 98, wherein the composition is for use in treating a subject in which, at or prior to the administration of the dose of cells:

the subject is or has been identified as having one or more cytogenetic abnormalities, optionally associated with high-risk CLL, optionally selected from among: complex karyotype, deletion of the long arm of chromosome 13 (del 13q), del 11, trisomy 12, del 17p, del 6q, and del 13q.14, optionally as detected by FISH;

the subject is or has been identified as having high-risk CLL; and/or

the subject is or has been identified as having extramedullary disease; and/or

the subject is or has been identified as having central nervous system (CNS) disease; and/or

the subject is an adult and/or is over at or about 30, 40, 50, 60, or 70 years of age.

100. The use of any of embodiments 97-99, wherein the composition is for use in treating a subject in which, prior to the administration of the dose of cells, the subject has been treated with two or more, optionally 3, 4, 5, 6, 7, 8, or 9 or more, therapies for the CLL, other than the lymphodepleting therapy and/or other than another dose of cells expressing the CAR.

101. The use of any of embodiments 97-100, wherein the composition is for use in treating a subject in which, prior to the administration of the dose of cells, the subject has been treated with two or more, optionally 3, 4, 5, 6, 7, 8, or 9 or more, therapies for the CLL, other than another dose of cells expressing the CAR or other than another dose of cells expressing the CAR and the preconditioning therapy.

102. The use of any of embodiments 97-101, wherein the composition is for use in treating a subject in which, prior to the administration of the dose of cells, the subject has been treated for the CLL with a kinase inhibitor, optionally an inhibitor of Btk, optionally ibrutinib.

103. The use of any of embodiments 97-102, wherein the composition is for use in treating a subject in which, prior to the administration of the dose of cells, the subject has been treated for the CLL with a monoclonal antibody that specifically binds to an antigen expressed by, or previously expressed by, cells of the CLL.

104. The use of any of embodiments 97-102, wherein the composition is for use in treating a subject in which, prior to the administration of the dose of cells, the subject has been treated for the CLL with venetoclax, a combination therapy comprising fludarabine and rituximab, radiation therapy and/or hematopoietic stem cell transplantation (HSCT).

105. The use of any of embodiments 97-102, wherein the composition is for use in treating a subject in which, at or immediately prior to the time of the administration of the dose of cells, the subject has relapsed following remission after treatment with, or become refractory to, one or more prior therapies for the CLL.

106. A composition comprising cells expressing a chimeric antigen receptor (CAR) that specifically binds to a target antigen of a non-Hodgkin lymphoma (NHL) for use in treating a subject having or suspected of having NHL, wherein the treating comprises administering to the subject a dose of cells expressing a chimeric antigen receptor (CAR) that specifically binds to a target antigen expressed by the NHL, wherein the treating comprises administering to the subject a dose of cells expressing the CAR, said dose (i) comprises (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg, and (ii) comprises a defined ratio of CD4⁺ cells expressing the CAR to CD8⁺ cells expressing the CAR and/or of CD4+ cells to CD8⁺ cells, which ratio optionally is approximately 1:1 or is between approximately 1:3 and approximately 3:1,

wherein, prior to the administration, the subject has been preconditioned with a lymphodepleting therapy comprising the administration of fludarabine.

107. A composition comprising cells expressing a chimeric antigen receptor (CAR) that specifically binds to a target antigen of a non-Hodgkin lymphoma (NHL) for use in treating a subject having or suspected of having NHL, wherein the treating comprises administering to the subject a dose of cells expressing the CAR, said dose (i) comprises (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells, and (ii) comprises a defined ratio of CD4⁺ cells expressing the CAR to CD8⁺ cells expressing the CAR and/or of CD4⁺ cells to CD8⁺ cells, which ratio optionally is approximately 1:1 or is between approximately 1:3 and approximately 3:1,

wherein, prior to the administration, the subject has been preconditioned with a lymphodepleting therapy comprising the administration of fludarabine.

108. The use of embodiment 106 or embodiment 107, wherein the composition is for use in treating a subject in which, at or prior to the administration of the dose of cells:

the subject is or has been identified as having one or more cytogenetic abnormalities, optionally associated with high-risk NHL;

the subject is or has been identified as having high-risk NHL; and/or

the NHL is selected from the group consisting of aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL); and/or

the subject is an adult and/or is over at or about 30, 40, 50, 60, or 70 years of age.

109. The use of any of embodiments 106-108, wherein the composition is for use in treating a subject in which, prior to the administration of the dose of cells, the subject has been treated with two or more, optionally 2, 3, or 4 or more, therapies for the NHL other than the lymphodepleting therapy and/or other than another dose of cells expressing the CAR.

110. The use of any of embodiments 106-109, wherein the composition is for use in treating a subject in which, at or immediately prior to the time of the administration of the dose of cells, the subject has relapsed following remission after treatment with, or become refractory to, one or more prior therapies for the NHL.

111. The use of any of embodiments 97-110, wherein the lymphodepleting therapy:

(i) further comprises administration of another chemotherapeutic agent other than the fludarabine, which optionally is cyclophosphamide;

(ii) is initiated at a time that is at least at or about 48 hours prior to or is between at or about 48 and at or about 96 hours prior to the administration of the cells; and

(iii) comprises the administration of cyclophosphamide at about 30-60 mg/kg, optionally once daily for one or two days, and/or the fludarabine at about 25 mg/m², daily for 3-5 days.

112. The use of any of embodiments 97-101, wherein the treating comprises administration of the cell dose and/or the lymphodepleting therapy via outpatient delivery.

113. The use of any of embodiments 97-112, wherein the composition and/or the dose of cells comprises a defined ratio of CD4+ cells expressing the CAR to CD8+ cells expressing the CAR and/or of CD4+ cells to CD8+ cells, which optionally is approximately 1:1 or is between approximately 1:3 and approximately 3:1.

114. The use of any of embodiments 97-113, wherein the composition and/or dose of cells is formulated for parenteral administration, optionally intravenous administration.

115. The use of any of embodiments 97-114, wherein the antigen is a B cell antigen, which optionally is CD19.

116. The use of any of embodiments 97-115, wherein the CAR comprises an scFv specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta.

117. The use of any of embodiments 97-116, wherein the CAR comprises a spacer and/or hinge region, each optionally derived from a human IgG.

118. The method of any of embodiments 97-117, wherein:

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain;

and wherein:
the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X1PPX2P, where X1 is glycine, cysteine or arginine and X2 is cysteine or threonine; and/or
the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or
the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or
the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 24.

### Vii. Definitions

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### VIII. EXAMPLES

The following examples are included for illustrative purposes only and are not intended to limit the scope of the invention.

### Example 1

### Treatment of Subjects with Relapsed or Refractory (R/R) CD19⁺ Chronic Lymphocytic Leukemia (CLL) with Chimeric Antigen-Receptor (CAR) Specific for CD19

Autologous T cells expressing a chimeric antigen-receptor (CAR) specific for CD19 were administered to thirteen (13) adult human subjects with relapsed or refractory (R/R) CD19⁺ chronic lymphocytic leukemia (CLL). The subjects ranged in age from forty (40) to seventy-three (73), with an average age of sixty-one (61). No subjects were excluded based on lymphopenia, circulating tumor, prior transplant, or test expansion. The group of subjects exhibited high-risk cytogenetics (10/13 (77%) del17p; 8/13 (62%) complex karyotype); 12/13 (92%) exhibited extramedullary disease. All subjects had been previously treated with one or more other therapies for CLL (with a median number of five (5), and a range of three (3) to nine (9), prior lines of treatment), including, in each case, ibrutinib (with seven (7) (54%) having being refractory and two (2) (15%) having been intolerant). Just prior to treatment, the median percentage of abnormal B cells in the bone marrow among all subjects was 66% (with a range of 0.4% to 90%).

The CAR included an scFv (in a VL-linker-VH orientation) specific for CD19, with variable regions derived from FMC63, an IgG hinge region, a transmembrane region, and intracellular signaling domains derived from human 41BB and CD3zeta. The construct further encoded a truncated EGFR (EGFRt), which served as a surrogate marker for CAR expression; the EGFRt-coding region was separated from the CAR sequence by a T2A skip sequence. Prior to administration of the cells, patients underwent leukapheresis; CD4+ and CD8+ populations were selected by immunoaffinity-based enrichment methods, transduced with a viral vector with the CAR construct, and expanded in culture over fifteen (15) days. For the manufacture of CAR+ T cells, CD8+ central memory T cell populations were engineered, except bulk CD8+ T cells were engineered in patients with severe lymphopenia resulting in a low CD8+ central memory T cells. No difference in clinical outcome was observed between patients who received CAR+ T cells manufactured from CD4+ T cells and either bulk CD8+ T cells or CD8+ central memory T cells.

Beginning at least forty-eight (48) (and up to ninety-six (96)) hours prior to CAR+ T cell infusion, subjects received a lymphodepleting chemotherapy with either (a) cyclophosphamide (Cy, 60 mg/kg) with or without etoposide (2/13 subjects), or (b) cyclophosphamide (Cy, 60 mg/kg) in combination with fludarabine (flu, 25 mg/m² daily for 3-5 days (cy/flu, 11/13 subjects).

Cells for administration generally were formulated at a CAR⁺ CD4⁺ T cell to CAR⁺ CD8⁺ T cell ratio of approximately 1:1. Therapeutic compositions were successfully produced for all subjects. For 1/13 subjects, fewer than the target dose (2×10⁶/kg CAR+) of cells were produced.

Subjects were infused with a composition having approximately a 1:1 ratio of CD8⁺ CAR⁺ T cells to CD4⁺ CAR-T cells, at one of three different dose levels (2×10⁵ (N=4) 2×10⁶ (N=8) or 2×10⁷ (N=1) CAR+ T cells per kilogram (kg) weight of the subject). Lymphodepleting therapy and T cell infusions were administered on an outpatient basis. After one CLL patient developed grade 4 CRS and grade 3 neurotoxicity after receiving 2×107 CAR+ T cells/kg, a maximal dose of 2×10⁶ CAR+ T cells/kg was selected for subsequent CLL patients.

Patients underwent whole-body imaging with a diagnostic quality CT scan before and 4 weeks after administration of the CAR+ T cell composition. Responses were determined by International Workshop on Chronic Lymphocytic Leukemia (IWCLL) response criteria (Hallek, et al., Blood 2008, Jun. 15; 111(12): 5446-5456; IWCLL (2008)). Nodal tumor bulk was assessed as the sum of the cross-sectional areas of the 6 largest index lymph nodes identified on a diagnostic quality CT scan. In some cases, whole body PEG imaging by Lugano criteria (Cheson et al., JCO Sep. 20, 2014 vol. 32 no. 27 3059-3067) also was performed.

Marrow response was assessed by bone marrow aspirate and biopsy obtained 4 weeks after CAR-T cell infusion in those with bone marrow disease prior to lymphodepletion and 4 weeks after administration of the CAR+ T cell composition. Morphology analysis and high-resolution flow cytometry were performed on the marrow, with conventional karyotyping and FISH in patients with an identified cytogenetic abnormality. IGH deep sequencing (Adaptive Biotechnologies) was performed on marrow from patients who had no detectable marrow disease by flow cytometry 4 weeks after CAR-T cell infusion and had an identified malignant clonal sequence before lymphodepletion. High-resolution flow cytometry was performed on blood 2 weeks, and 1, 2, 3, 6 and 12 months after CAR-T cell infusion.

Toxicity was graded using the National Cancer Institute—Common Toxicity Criteria version 4.03 (NCI-CTCAE v4.03), except cytokine release syndrome was graded as described in Lee et al, Blood. 2014; 124(2):188-95.

Among the eleven (11) subjects preconditioned with the combination of cyclophosphamide and fludarabine (cy/flu), the objective response rate (ORR) was 91% (10/11 subjects), and 10/11 subjects (91%) were observed to be negative for tumor cells in the bone marrow as measured by flow cytometry, and five (5) of the 11 (45%) were observed to achieve complete remission (CR) as measured by Lugano criteria. Among those two subjects not having been preconditioned with cy/flu, the ORR was 50% (1/2 subjects), with 1 of the 2 subjects observed as negative for tumor cells in the bone marrow as measured by flow cytometry, but not observed to have achieved CR by Lugano criteria. In those two subjects assessed as achieving partial remission (PR), max 17-18 mm lymph nodes were observed. In four (4) of the subjects having achieved CR, IGH deep sequencing of bone marrow was carried out after treatment. In 4/4 (100%) of these subjects, the index clone was not detected in the bone marrow.

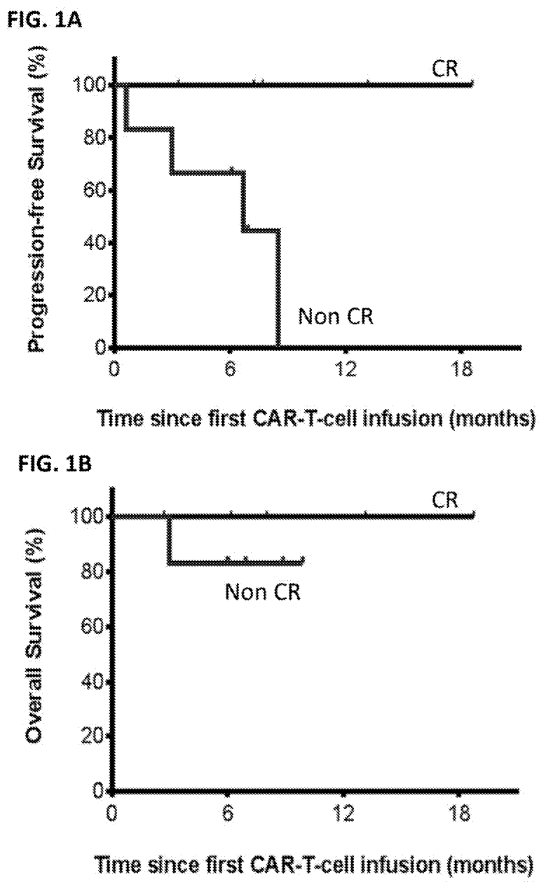

Progression-free survival (PFS) and overall survival (OS) among the 11 subjects preconditioned with cyclophosphamide are shown in FIGS. 1A and 1B, with separate curves shown for the group of subjects who achieved CR, versus those who did not. Median PFS was not reached in the subjects who had achieved CR; PFS ranged from 3+ to 19+ months from first CAR+ T cell infusion.

In this study, twenty-three percent (23%) of the 13 subjects exhibited severe cytokine release syndrome (CRS), assessed according to Lee et al, Blood. 2014; 124(2):188-95; 23% exhibited grade 3 or higher neurotoxicity.

The results demonstrated that administration of the CD19-specific CAR+-T cells at defined CD4+/CD8+ ratios resulted in durable CR in a majority of the subjects with high-risk relapsed/refractory CLL.

### Example 2

### Treatment of Subjects with Non-Hodgkin Lymphoma (NHL) with Chimeric Antigen-Receptor (CAR) Specific for CD19

Autologous T cells expressing a chimeric antigen-receptor (CAR) specific for CD19 were administered to forty-one (41) adult human subjects with CD19⁺ non-Hodgkin lymphoma (NHL). The group of subject ranged in age from twenty-eight (28) to seventy (70), with an average age of fifty-six (56). No subjects were excluded based on lymphopenia, circulating tumor, prior transplant, or test expansion. The group of subjects exhibited a number of disease types including aggressive NHL (30/41 subjects, including diffuse large B-cell lymphoma (DLBCL), primary mediastinal large B cell lymphoma (PMBCL), T cell/histocyte-rich large B cell lymphoma (TCHRBCL), and Burkitt lymphoma), mantle cell lymphoma (MCL; 5/41 subjects), and follicular lymphoma (FL; 6/41 subjects). All subjects had been previously treated, with a median number of four (4) prior treatments and a range of one (1) to eleven (11) prior treatments, among all subjects, and twenty-seven (27) subjects having been treated with greater than or equal to 4 prior therapies. Nineteen (19) of the 41 subjects had received prior auto- and/or allo-hematopoietic stem cell transplantation (HSCT).

The CAR included an anti-CD19 scFv (in a VL-linker-VH orientation) with variable regions derived from FMC63, an IgG hinge region, a transmembrane region derived from human CD28, and intracellular signaling domains derived from human 41BB and CD3zeta. The construct further encoded a truncated EGFR (EGFRt), which served as a surrogate marker for CAR expression; the EGFRt-coding region was separated from the CAR sequence by a T2A skip sequence. Prior to administration of the cells, patients underwent leukapheresis; CD4+ and CD8+ populations were selected by immunoaffinity-based enrichment methods, transduced with a viral vector with the CAR construct, and expanded in culture over 15 days. Cells for administration generally were formulated at a CD4⁺ T cell to CD8⁺ T cell ratio of approximately 1:1, prior to administration. Therapeutic compositions were successfully produced for all subjects.

Beginning at least forty-eight (48) (and up to ninety-six (96)) hours prior to CAR+ T cell infusion, 39 subjects received a lymphodepleting chemotherapy with either (a) cyclophosphamide (Cy, 60 mg/kg) with or without etoposide (12/39 subjects), or (b) cyclophosphamide (Cy, 60 mg/kg) in combination with fludarabine (flu, 25 mg/m² daily for 3-5 days (cy/flu, 27/39 subjects).

Subjects were infused with a composition having approximately a 1:1 ratio of CD8⁺ CAR⁺ T cells to CD4⁺ CAR-T cells, at one of three different dose levels (2×10⁵ (N=5) 2×10⁶ (N=27) or 2×10⁷ (N=9) CAR+ T cells per kilogram (kg) weight of the subject). Lymphodepleting therapy and T cell infusions were administered out on an outpatient basis. In this study, the maximum tolerated dose was dose level 2 (2×10⁶ CAR+ T cells per kg).

Among the twenty-seven (27) subjects preconditioned with cyclophosphamide and fludarabine (cy/flu), the objective response rate (ORR) was 74% (20/27 subjects). Twelve (12) of the 27 (44%) were observed to achieve complete remission (CR).

Among the 27 subjects preconditioned with cy/flu, twenty (20) subjects, across all disease subtypes outlined above, were administered dose level 2 (2×10⁶ CAR+ T cells per kg). Of these subjects, the ORR was 80% (16/20) and 10 of the 20 subjects (50%) were observed to achieve CR. Sixteen (16) of the thirty (30) subjects with aggressive lymphoma were preconditioned with the cy/flu therapy and administered dose level 2. Among these 16 subjects, the ORR was 81% (13/16 subjects) and eight (8) subjects (50%) achieved CR. Two (2) of the 6 subjects with FL were preconditioned with cy/flu and administered dose level 2. Among these two subjects, the ORR was 50% (1/2) and 1 subject (50%) achieved CR. Two (2) of the five (5) subjects with MCL were preconditioned with cy/flu and administered dose level 2. Among these subjects, the ORR was 100% (2/2) and 1 subject (50%) achieved CR.

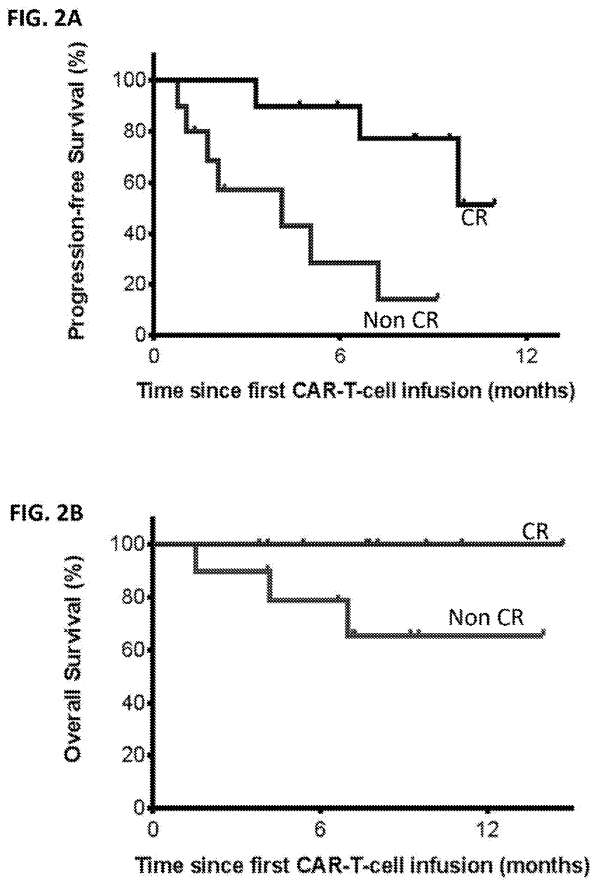

Progression-free survival (PFS) and overall survival (OS) among the 20 subjects preconditioned with cyclophosphamide and fludarabine (cy/flu) and administered 2×10⁶ CAR+ T cells per kg are shown in FIGS. 2A and 2B, with separate curves for subjects who achieved CR, versus those who did not. Median PFS was not reached in the subjects who achieved CR; PFS ranged from 3 to 11+ months from first CAR+ T cell infusion. Median PFS was 4.1 months in subjects who did not achieve CR.

In this study, seventeen percent (17%) of subjects at all dose levels of subjects exhibited severe cytokine release syndrome (CRS), assessed according to Lee et al, Blood. 2014; 124(2):188-95; five percent (5%, 2/41) exhibited grade 5 CRS, and 20% exhibited grade 3 or higher neurotoxicity. Among those subjects who were administered 2×10⁶ CAR+ T cells per kg following preconditioning with cy/flu (20 subjects), only ten percent (10%) exhibited severe cytokine release syndrome (CRS) and only 10% exhibited grade 3 or higher neurotoxicity.

### Example 3

### Treatment of Subjects with Relapsed or Refractory (R/R) CD19⁺ CLL with Chimeric Antigen-Receptor (CAR) Specific for CD19: Additional Patients

In an extension of the study described in Example 1, additional subjects with relapsed or refractory (R/R) CD19+ chronic lymphocytic leukemia (CLL) were evaluated. Eighteen (18) adult human subjects were administered the autologous T cells expressing a chimeric antigen-receptor (CAR) specific for CD19 and evaluated as described below.

The subjects ranged in age from forty (40) to seventy-three (73), with a median age of sixty (60). Twelve (12) subjects had complex karyotype and eleven (11) subjects had 17p deletion. All subjects had extramedullary disease and two (2) had central nervous system (CNS) disease. All subjects had been previously treated with one or more other therapies for CLL (with a median number of five (5), and a range of three (3) to nine (9), prior lines of treatment), including, in each case, ibrutinib (with eleven (11) (61%) having been refractory; three (3) (17%) having been intolerant). Three (3) (17%) subjects had failed prior allogeneic stem cell transplant and four (4) (22%) subjects were refractory to venetoclax. All subjects also were refractory to or had relapsed after receiving a depleting chemotherapy regimen containing fludarabine and rituximab. Just prior to treatment, the median percentage of abnormal B cells in the bone marrow among all subjects was 77% (with a range of 0.4% to 90%).

Autologous CAR− T cells were manufactured for all subjects and administered to subjects as described in Example 1, but additional subjects were treated. 16/18 subjects received a cell composition with a CAR⁺ CD4⁺ T cell to CAR⁺ CD8⁺ T cell ratio of approximately 1:1. Subjects were infused with the cell composition at the different dose levels as follows: (2×10⁵ (N=4); 2×10⁶ (N=13); or 2×10⁷ (N=1) CAR+ T cells per kilogram (kg) weight of the subject).

Prior to CAR-T cell infusion, the treated subjects received a lymphodepleting chemotherapy with either (a) cyclophosphamide 30-60 mg/kg×1 in combination with fludarabine 25 mg/m²/day×3 days (cy/flu, 15/18 subjects), (b) fludarabine 25 mg/m²/day×3 days (flu, 2/18 subjects) or (c) cyclophosphamide 60 mg/kg (cy, 1/18 subjects). Four (4) subjects with persistent disease received a second cycle of lymphodepletion chemotherapy and CAR+ T cells at a 10-fold higher dose than the first infusion.

Subjects were assessed 4 weeks after the last CAR+ T cell infusion as described in Example 1.

Seventeen subjects completed response and toxicity assessment. Among the seventeen (17) subjects assessed, the objective response rate (ORR) was 76% (13/17 subjects; 8 with partial remission (PR) and 5 with complete remission (CR)). Two (2) subjects with PR based on the lymph node size criteria (IWCLL 2008) had negative PET scans after therapy. Among thirteen (13) ibrutinib-refractory or intolerant subjects, the ORR was 77% (10/13 subjects, 7 PR and 3 CR). Among four (4) venetoclax-refractory subjects, the ORR was 50% (2/4 subjects, 2 PR). Among those three (3) subjects not having been preconditioned with the combination of cy/flu, the ORR was 33% (1/3 subjects).

At day 28, among the thirteen (13) subjects who received cy/flu lymphodepletion and 2×10⁵ or 2×10⁶ CAR+ T cells/kg, eleven (11) (85%) exhibited complete elimination of marrow disease by flow cytometry; 10/13 (77%) with nodal disease exhibited PR or CR; 1/13 (8%) had a mixed response; and 2/13 (15%) exhibited progressive disease (PD),

In four (4) of the subjects having achieved CR, IGH deep sequencing of bone marrow was performed. No malignant sequences were detected in 4/4 (100%) of these subjects.

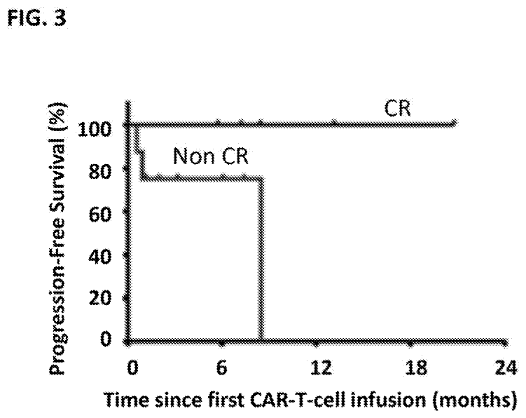

Progression-free survival (PFS) among the thirteen (13) subjects who received cy/flu lymphodepletion and 2×10⁵ or 2×10⁶ CAR+ T cells/kg, are shown in FIG. 3, with separate curves shown for the group of subjects who achieved CR, versus those who did not. Overall survival (OS) was 100% in this group. No subjects who achieved CR had relapsed or died after a median follow-up of 8.4 months. The results also showed that subjects who had achieved CR exhibited a higher peak percentage of CD8+(p=0.006), but not CD4+ CAR+ T cells in blood. Robust CAR-T cell expansion was seen in some non-responders.

The result further demonstrated that administration of the CD19-specific CAR+-T cells at defined CD4+/CD8+ ratios resulted in a high response rate and durable CR in a majority of the subjects with high-risk relapsed/refractory CLL, such as patients who have failed ibrutinib treatment.

### Example 4

### Treatment of Subjects with Relapsed or Refractory (R/R) CD19⁺ CLL with Chimeric Antigen-Receptor (CAR) Specific for CD19: Further Patients

A. Subjects and Treatment

In an extension of the studies described in Examples 1 and 3, additional subjects with relapsed or refractory (R/R) CD19+ chronic lymphocytic leukemia (CLL) were evaluated. In total, twenty-four (24) adult human subjects, who had received previous therapies, were administered the autologous T cells expressing a chimeric antigen-receptor (CAR) specific for CD19, following lymphodepletion, and evaluated as described in Examples 1 and 3 and below.

As described in Table 4, the subjects ranged in age from 40 to 73, with a median age of 61. Sixteen (16) subjects had complex karyotype and 14 subjects had 17p deletion. Eight (8) subjects had high-risk histology. Twenty-three (23) subjects had extramedullary disease. All subjects had been previously treated with one or more other therapies for CLL, with a median of 5 (range 3-9) prior lines of treatment, which included, in each case, treatment with ibrutinib (with 19 (79%) having been refractory; 3 (13%) having been intolerant). Nine (9) of the 18 ibrutinib-refractory subjects had a BTK or PLCG2 mutation (50%; BTK, n=7; PLCG2, n=2). Ibrutinib was discontinued in all subjects before lymphodepletion. Four (4) (17%) subjects had failed prior allogeneic stem cell transplant and 6 (25%) subjects were refractory to venetoclax. Just prior to treatment, the median percentage of abnormal B cells in the bone marrow among all subjects was 61.6%% (range 0.0%-96%). Just prior to treatment, the median abnormal B cell count in the blood among all subjects was 1.1×10³/μL (range of 0.0-76.68×10³/μL).

TABLE 4
High-Risk CLL Population Characteristics

| Characteristic | N = 24 | |
|---|---|---|
| Age at infusion, median [range], years | 61 | [40-73] |
| Prior lines of therapy, median [range] | 5 | [3-9] |
| Prior allogeneic HCT | 4 | (17%) |
| Prior Ibrutinib, median duration 13 mo. | 24 | (100%) |
| [range 0.75-39 mo.] | | |
| Ibrutinib-refractory | 19 | (79%) |
| BTK or PLCG2 mutation | 9/19 | (47%) |
| Ibrutinib-intolerant | 3 | (13%) |
| Venetoclax-refractory | 6 | (25%) |
| Refractory or relapsed after fludarabine with | 23 | (96%) |
| rituximab | | |
| Failed bendamustine/rituximab | 1 | |
| High-risk cytogenetics, N (%) | 23 | (96%) |
| Complex karyotype | 16 | (67%) |
| 17p del | 14 | (58%) |
| High-risk histology (Richter's/IPC/PLL), N (%) | 8 | (33%) |
| Marrow abnormal B cells before lymphodepletion | 64.5 | [0-96] |
| chemotherapy, median [range], % | | |
| Extramedullary disease, N (%) | 23 | (96%) |
| Cross-sectional area, median [range], mm² | 3093 | [546-20406] |
| FDG-avid disease on PET, N (%) | 14/15 | (93%) |
| Maximum SUV, median [range] | 7.1 | [3.4-27.5] |
| Active CNS disease | 2 | (8%) |
| Measureable disease by CT and FDG-avid disease | 14/15 | (93%) |
| on PET, N (%) | | |
| Maximum SUV, median [range] | 7.1 | [3.4-27.5] |

Autologous CAR-T cells were manufactured for all subjects and administered to subjects as described in Examples 1 and 3, but with additional subjects treated. Twenty-two (22) of the 24 subjects received a cell composition with a CAR⁺ CD4⁺ T cell to CAR⁺ CD8⁺ T cell ratio of approximately 1:1, and 2 patients received less than the target CD8+ CAR-T cell dose (58.5% and 56.3%). Subjects were infused with the cell composition the different dose levels as follows: 2×10⁵ (N=4); 2×10⁶ (N=19); or 2×10⁷ (N=1) CAR+ T cells per kilogram (kg) weight of the subject. Prior to CAR-T cell infusion, 21 subjects received lymphodepleting chemotherapy as outlined in Table 5. The treated subjects received a lymphodepleting chemotherapy received either (a) cyclophosphamide 30-60 mg/kg (1-2 g/m²)×1 in combination with fludarabine 25 mg/m²/day×3 days (cy/flu, 18/24 subjects), (b) cyclophosphamide 60 mg/kg (1-2 g/m²)×1 in combination with fludarabine 25 mg/m²/day×5 days (cy/flu, 1/24 subjects), (c) fludarabine 25 mg/m²/day×3 days (flu, 2/24 subjects), (d) cyclophosphamide 60 mg/kg (cy, 1/24 subjects) or (e) 500 mg/m²×3 with fludarabine 25 mg/m²/day×3 days (cy/flu, 2/24 subjects). A total of 15 patients in the study received Cy/Flu lymphodepletion and 2×10⁶ CAR+ T cells.

TABLE 5
Lymphodepletion and Immunotherapy in High-Risk CLL Patients

| Lymphodepleting chemotherapy | | |
|---|---|---|
| Cyclophosphamide/fludarabine (Cy/Flu) | 21 | (87%)* |
| Non-Cy/Flu | 3 | (13%) |
| CAR-T cell manufacturing | | |
| CD4⁺ and CD8⁺ central memory | 7 | (29%) |
| CD4⁺ and CD8⁺ bulk | 17 | (71%) |
| CD19 CAR-T cell dose level | | |
| DL1 (2 × 10⁵ EGFRt⁺ cells/kg) | 3 | (13%) |
| DL2 (2 × 10⁶ EGFRt⁺ cells/kg) | 20 | (83%) |
| DL3 (2 × 10⁷ EGFRt⁺ cells/kg) | 1 | (4%) |
| Single cycle | 18 | (75%) |
| Second cycle for residual disease or relapse | 6 | (25%) |

*1 patient died prior to restaging

Six (6) subjects with persistent disease received a second cycle of lymphodepletion chemotherapy and CAR+ T cell infusion at the same (N=1) or at a 10-fold higher dose (N=5) than the first infusion.

During the 3 weeks between leukapheresis and lymphodepletion chemotherapy, 6 patients required high-dose corticosteroids to control progressive disease and 2 others required treatment for tumor-associated hypercalcemia.

B. Response to Treatment

Subjects were assessed for response 4 weeks after the last CAR+ T cell infusion as described in Example 1. The assessment of twenty-one (21) subjects receiving Cy/Flu lymphodepletion demonstrated high response rates in high-risk CLL patients at four weeks post-infusion as shown in Table 8. Responses were measured in subjects by: (a) bone marrow analysis; (b) PET-CT; and (c) International Workshop Group on CLL (IWCLL) criteria.

TABLE 8
Response Rates in Subjects at Four Weeks Post-infusion

| | | | Cy/Flu lymphodepletion (N = 21)* | | | |
|---|---|---|---|---|---|---|
| | Non-Cy/Flu | | All patients | | Ibrutinib-refractory | |
| Lymphodepletion | (N = 3 restaged) | | (N = 19 restaged) | | (N = 16 restaged) | |
| Dose Level | All Doses | | DL 1, 2 | | DL 1, 2 | |
| IWCLL restaging | N = 3 | | N = 19 | | N = 16 | |
| ORR (at 4 weeks) | 1/3 | (33%) | 14/19 | (74%)** | 11/16 | (69%)** |
| CR (at 4 weeks) | 0/3 | (0%) | 4/19 | (21%) | 4/16 | (25%) |
| BM disease at baseline | N = 3 | | N = 17 | | N = 14 | |
| Flow-negative | 1/3 | (33%) | 15/17 | (88%) | 12/14 | (86%) |
| (at 4 weeks) | | | | | | |
| PET-avid disease at baseline | N = 1 | | N = 11 | | N = 11 | |
| ORR (at 4 weeks) | 0/1 | (0%) | 8/11 | (73%)** | 8/11 | (73%)** |
| CR (at 4 weeks) | 0/1 | (0%) | 7/11 | (64%)** | 7/11 | (64%)** |

**One additional patient reported as SD (IWCLL, PET) at 4 weeks achieved PR (IWCLL, max 16 mm) and CR (PET) 8 weeks after CAR-T cell infusion without additional therapy
*1 patient died prior to restaging

1. CAR-T cell Expansion and Persistence

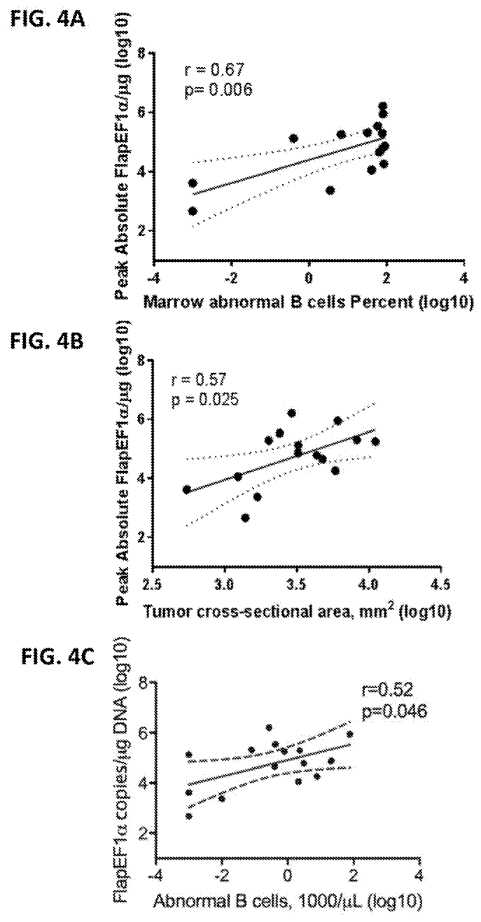

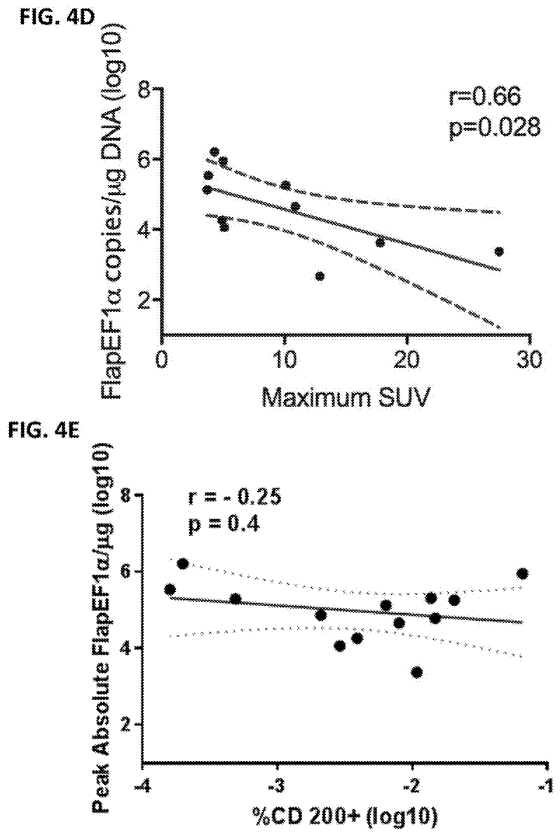

After CAR-T cell infusion, CAR-T cells were detected in blood by flow cytometry in all patients to assess expansion and persistence. Among subjects who received cyclophosphamide/fludarabine (Cy/Flu) lymphodepletion and were infused with 2×10⁶ CAR-T cells, greater CAR-T cell expansion was positively correlated with the, percentage of abnormal B cells present in bone marrow (r=0.67, p=0.006), tumor cross-sectional area (r=0.57, p=0.025) and absolute abnormal B cell count in blood as shown in FIG. 4A-4C, respectively. In addition, there was an inverse correlation between CAR-T cell expansion and the SUVmax in those subjects with FDG-avid disease on pretreatment PEG scans (FIG. 4D). CAR-T cells were detected by QPCR in blood at ≥6 months in all patients (n=11) that were evaluated and did not undergo subsequent allogeneic HCT.

In the subjects who received Cy/Flu lymphodepletion and were infused with 2×10⁶ CAR-T cells, CAR-T cell expansion was inversely correlated with the immune checkpoint biomarker CD200 (r=−0.25, p=0.4) as shown in FIG. 4E. CAR-T cell expansion was also inversely correlated with immune checkpoint biomarkers PDL1 and PDL2.

2. Lymph Node Response Rate

Twenty three (23) patents were evaluated for lymph node response 4 weeks after CAR-T cell infusion by IWCLL criteria. Among the 23 restaged patients, the overall response rate (ORR) at 4 weeks after CAR+ T cell infusion by IWCLL lymph node criteria was 70% (16/23). Among the 3 patients who did not receive Cy/Flu lymphodepletion, 1 cleared marrow disease, 1 had a partial response (PR), and all developed progressive disease.

Among 19 subjects who completed IWCLL analysis and received Cy/Flu lymphodepletion and a single CD19 CAR-T cell infusion at ≤2×10⁶ CAR-T cells/kg, a lymph node response by IWCLL criteria was observed in 74% of patients (14/19) [95% confidence interval (CI): 49-91%]: 21% (4/19) CR; 53% (10/19) PR. Similar response rates were observed when considering only the 16 patients that were ibrutinib-refractory: 69% (11/16) ORR [95% CI: 41-89%]; 25% (4/26) CR).

Where feasible, lymph node response was also assessed by PET imaging 4 weeks after CAR-T cell infusion. The CR rate in ibrutinib-refractory patients after PET-CT restaging was 64% (7/11) [95% CI: 31-89%], with a Deauville score of 1-2. More patients were staged as CR by PET-CT than by IWCLL (64% vs. 25%). Four (4) of 5 patients who achieved PR by IWCLL and underwent PET imaging had no FDG-acid disease after CAR-T cell infusion. One additional patient with stable disease according to PET-CT, 4 weeks after CAR-T cell infusion, subsequently achieved CR on a follow-up PET-CT, 8 weeks later.

3. Assessment of Malignant IGH Sequences from Marrow

Twenty-two of 24 patients had marrow disease before treatment and 21 patients had a bone marrow evaluation 4 weeks after CAR+ T cell administration. Seventeen of 21 patients (81%) had no marrow disease detected by high resolution flow cytometry. Of the subjects who received Cy/Flu lymphodepeletion, administration of ≤2×10⁶ CAR-T cells/kg and had bone marrow involvement prior to therapy, fifteen out of seventeen (15/17) exhibited clearance of bone marrow disease by high-resolution flow cytometry (88% [95% CI: 64-99%]). The flow-negative marrow response rate in the subset of ibrutinib-refractory patients was similar (12/14; 86% [95% CI: 57-98%]). FISH and conventional karyotyping did not identify residual CLL in patients without detectable disease by flow cytometry; however, 2 patients had abnormalities considered to be due to the effects of prior chemotherapy on the myeloid lineage and one had a persistent constitutional translocation.

Twelve patients who cleared marrow by flow cytometry, after Cy/Flu and ≤2×10⁶ CAR-T cells/kg infusion, also had an identified clonal malignant IGH sequence in CLL cells before treatment. Seven of the 12 (7/12; 58%) subjects, identified as having a clonal malignant IGH sequence, exhibited clearance of bone marrow disease by IGH sequencing 4 weeks after CAR-T cell infusion.

Of the 6 patients that received a second cycle of lymphodepletion chemotherapy and CAR-T cell infusion at the same (n=1) or 10-fold higher dose (n=5), 2 (2/6; 33%) achieved CR (PET-CT) and eliminated bone marrow disease by flow cytometry and IGH sequencing. Four of the 6 patients (4/6; 67%) developed CRS (2 grade ≥3) and one developed reversible neurologic toxicity (grade 3) after the second CAR-T cell infusion.

A subset of patients who achieved PR by IWCLL at initial restaging at 4 weeks had no FDG-avid disease by PET-CT criteria (4/5), and/or had no detectable malignant IGH sequence in marrow (4/6). This observation is consistent with a finding that the IWCLL criteria might underestimate the response achieved with CAR-T cells, which is further supported by the survival data described below, measured by IWCLL response, showing an equivalent PFS in patients who achieved PR or CR, and ongoing tumor regression after initial response in one patient.

4. Progression Free Survival and Overall Survival

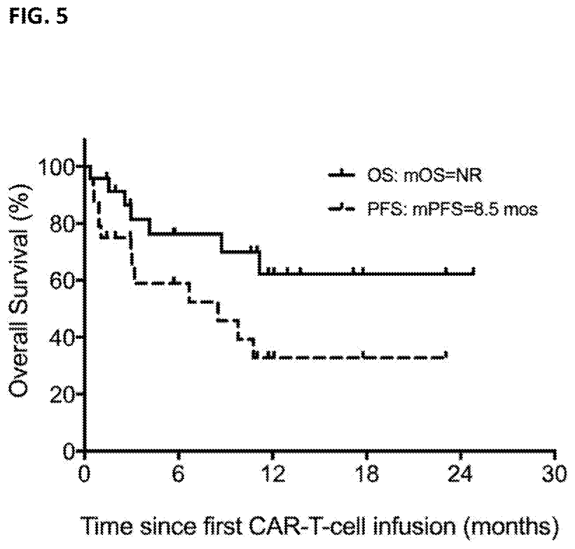

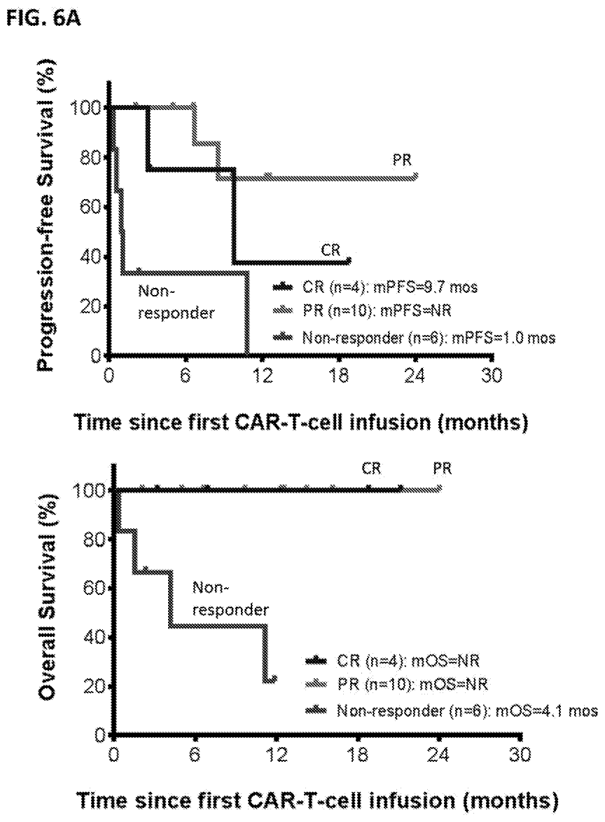

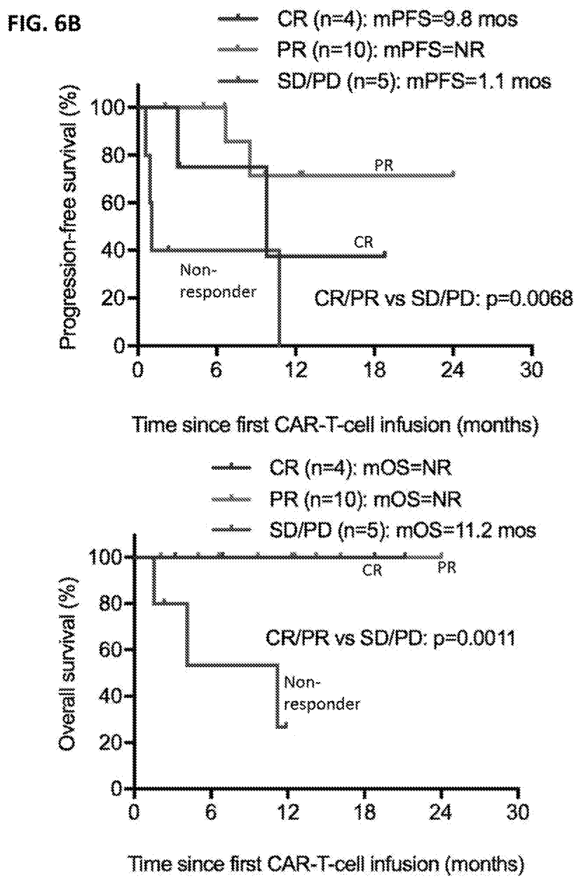

Progression free survival (PFS) and overall survival (OS) for all CLL patients are shown in FIG. 5. A lymph node response (CR/PR by IWCLL) was associated with longer PFS and OS compared to failure to respond (SD/PD). Progression free survival (PFS) and overall survival (OS), as measured by IWCLL nodal response criteria, among the subjects who received Cy/Flu lymphodepletion and 2×10⁵ or 2×10⁶ CAR-T cells/kg, are shown in FIG. 6A. FIG. 6B also depicts PFS and OS as measured by IWCLL among the same group of subjects as FIG. 6A, except excluding one patient that died prior to restaging. For both FIGS. 6A and 6B, separate curves are shown for the group of subjects who achieved CR, subjects who achieved PR, and non-responders, and indicate that patients who achieved PR by IWCLL criteria did not have inferior PFS and OS compared to those who achieved CR. OS was 100% in subjects who achieved CR or PR. No subjects who achieved CR or PR died within 24 months of the first CAR-T cell infusion.

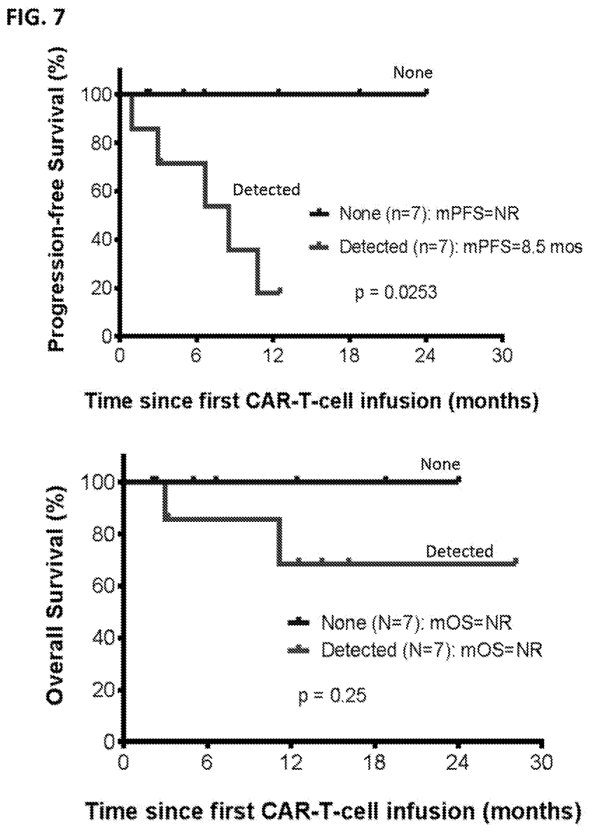

The survival of patients who cleared marrow by flow cytometry was analyzed for the presence (detected) or absence (none) of malignant IGH sequences in marrow 4 weeks after CAR-T cell infusion. Among 14 subjects, 7 subjects had malignant sequences detected by IGH deep sequencing. PFS and OS are shown in FIG. 7. PFS and OS were reduced in the 7 subjects with malignant sequences, compared to the 7 subjects without malignant sequences. Thus, independent of the IWCLL response, patients who were negative for malignant IGH sequences had better PFS compared to those with persistent malignant IGH sequences. Median OS was not reached in either group. The positive effect of marrow clearance by IGH sequencing on outcome was also observed when the analysis was restricted to PFS patients who responded (CR/PR) by IWCLL criteria (p=0.063, mPFS for IGHseq-positive=8.5 months, mPFS for IGHseq-negative not reached).

The results further demonstrated that administration of the CD19-specific CAR+-T cells at defined CD4⁺/CD8⁺ ratios resulted in a high response rate and durable CR in a majority of the subjects with high-risk relapsed/refractory CLL, such as patients who have failed ibrutinib and/or venetoclax treatment. Additionally, the results indicated that the detection of malignant sequences by IGH deep sequencing of bone marrow after CAR T-cell therapy may provide early signs of durable responses.

C. Toxicity

Twenty-four (24) subjects were assessed for symptoms of cytokine release syndrome (CRS) and neurotoxicity as shown in Table 6 and Table 7, respectively.

TABLE 6
CRS in CLL Subjects after CAR T cell therapy

| CRS | | CLL (N = 24) | |
|---|---|---|---|
| CRS grade | 0 | 4 | (17%) |
| (Lee et al, Blood. | 1 | 8 | (33%) |
| 2014; 124(2): 188-95) | 2 | 10 | (42%) |
| | 3 | 0 | (0%) |
| | 4 | 1 | (4%) |
| | 5 | 1 | (4%) |

TABLE 7
Neurotoxicity in CLL Subjects after CAR T cell therapy

| Neurotoxicity | | CLL (N = 24) | |
|---|---|---|---|
| Neurotoxicity grade | 0 | 16 | (67%) |
| (CTCAE v4.03) | 1 | 0 | (0%) |
| | 2 | 2 | (8%) |
| | 3 | 5 | (21%) |
| | 4 | 0 | (0%) |
| | 5 | 1 | (4%) |

Tocilizumab (4-8 mg/kg I.V.) and dexamethasone (10 mg bid I.V.) were administered to patients who either required management in the intensive care unit (ICU) or were under evaluation for ICU care. Intervention was initiated in patients with grade 2-3 cytokine release syndrome (Lee et al, Blood, 2014) that were not responding to intravenous fluids and/or low dose vasopressor support and grade 2-3 neurotoxicity. Of treated patients, 1 patient progressed to grade 3-4 CRS from day 4 that was refractory to tocilizumab and dexamethasone, developed cerebral edema on day 9 that was refractory to siltuximab and mannitol and died 11 days after CAR-T cell infusion. No other patients developed greater than grade 2 CRS and only 4 patients developed grade 3 neurotoxicity. Only 6/24 patients exhibited clinical symptoms sufficiently severe to require an intervention therapy, and CRS and neurotoxicity resolved in all patients treated according to these criteria, with the exception of the patient with fatal cerebral edema.

### Example 5

### Factors Correlating with Response and/or Toxicity Based on Peak CAR T Cell Expansion in High-Risk CLL Patients

Following the evaluation of expansion and response to CD19 CAR-T cell therapy for relapsed or refractory (R/R) CD19⁺ chronic lymphocytic leukemia (CLL), as described above in Examples 1, 3, and 4, subjects with relapsed or refractory (R/R) CD19+ chronic lymphocytic leukemia (CLL) were evaluated for expansion and response.

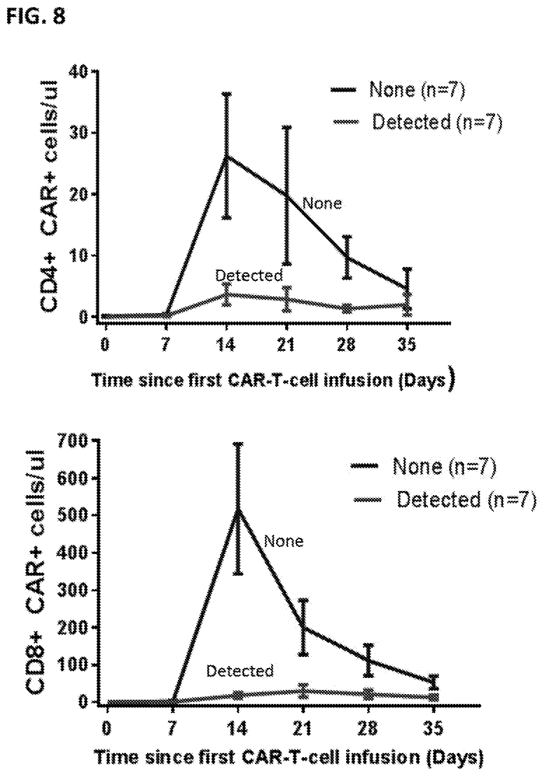

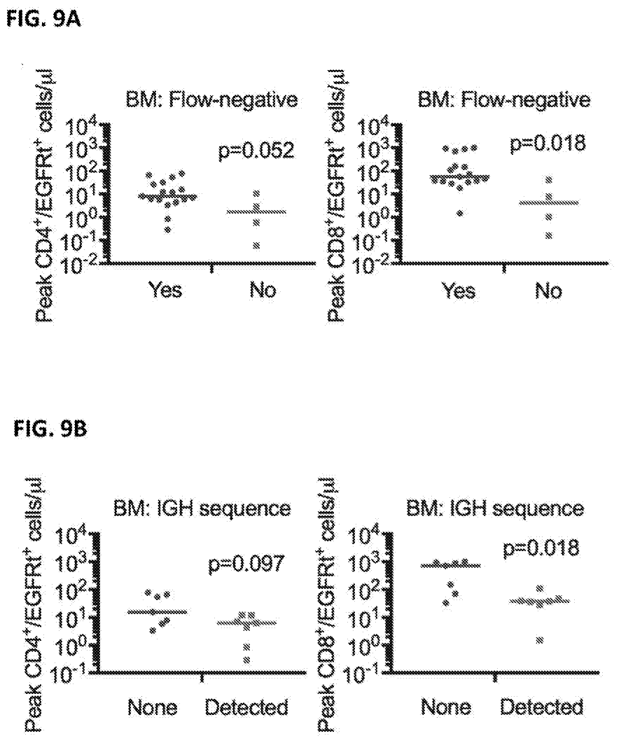

Elevated peak CD4+ or CD8+ CAR-T cell counts after infusion were associated with better bone marrow responses in high-risk CLL subjects as assessed by the presence (detected) or absence (none) of malignant IGH sequences in marrow 4 weeks after CAR-T cell infusion (FIG. 8). Peak CD4⁺/EGFRt⁺ and CD8⁺/EGFRt⁺ CAR-T cell counts were higher in patients who ultimately cleared bone marrow by flow cytometry compared to those who failed to eliminate CLL from marrow (FIG. 9A), and also higher in those who achieved CR by flow cytometry and had no malignant IGH sequences detected in marrow compared to those who had CR by flow cytometry with detectable malignant IGH sequences (FIG. 9B).

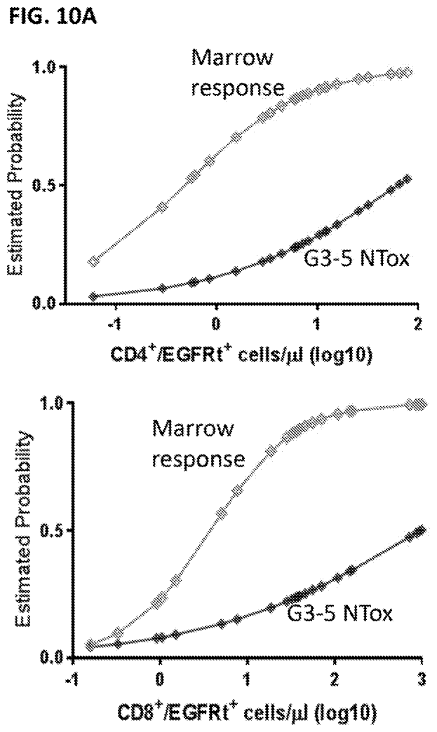

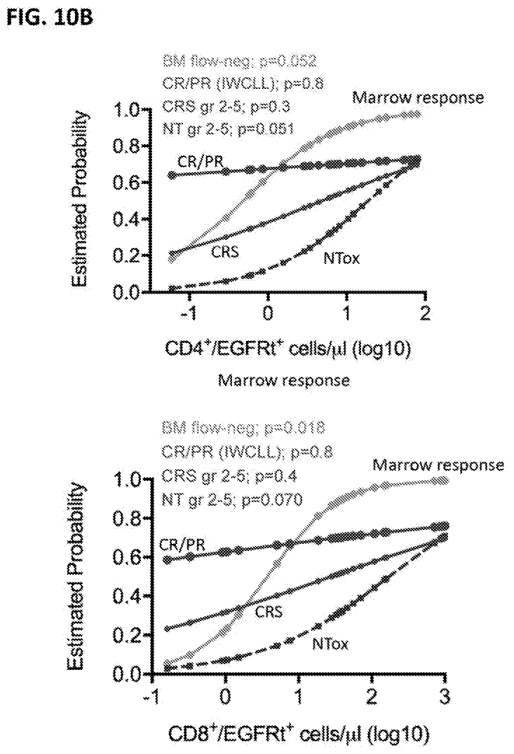

Probability curves depicting the probability estimated by logistic regression of clinical outcomes associated with peak CD4+/EGFRt⁺ and CD8⁺/EGFRt⁺ CAR-T cell counts in blood were generated and are depicted in FIGS. 10A and 10B. An estimated probability curve of response and an estimated probability of developing Grade 3-5 neurotoxicity were constructed based on the number of CD4+/EGFRt⁺ or CD8+/EGFRt⁺ CAR-T cells in the blood (FIG. 10A). An estimated probability curve of response and an estimated probability of developing Grade 2-5 neurotoxicity or CRS were constructed based on the number of CD4⁺/EGFRt⁺ or CD8⁺/EGFRt⁺ CAR-T cells in the blood (FIG. 10B). Generally, as the number of CAR-T cells increased, the probability of bone marrow response increased then plateaued while the probability of developing a toxicity, Grade 3-5 neurotoxicity (FIG. 10A) or Grade 2-5 neurotoxicity or CRS (FIG. 10B) increased. These curves demonstrate a therapeutic window of peak CD4+/EGFRt⁺ and CD8⁺/EGFRt⁺ CAR-T cell counts in which marrow clearance might be achieved in most patients without a high risk of neurotoxicity or CRS.

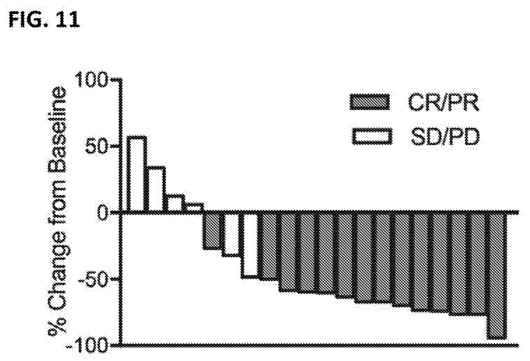

Robust anti-tumor activity, as determined by changes in cross-sectional area of 6 largest lymph nodes on CT scan by IWCLL imaging criteria, was seen in a subset of patients with large lymph node tumor burdens, including those with Richter's transformation (FIG. 11), however patients with higher lymph node tumor bulk were overall less likely to respond to CAR-T cells (CR vs PR vs NR by IWCLL, p=0.098), as were those with fewer prior therapies (CR/PR vs NR by IWCLL; 5.5 vs 4; p=0.04). This suggests that bulky and aggressive nodal disease might be less amenable to CAR-T cell therapy. The relationship between the peak CAR-T cell count in blood and the probability of a response in lymph nodes was less robust than that noted for marrow, but higher peak CD3⁺/EGFRt⁺ CAR-T cell counts in blood were associated with reduced risk of disease progression and death in high-risk CLL patients (HR 0.56, 95% confidence interval 0.34-0.93, p=0.025).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO. | SEQUENCE | DESCRIPTION |
| 1 | ESKYGPPCPPCP | spacer |
| | | (IgG4hinge) |
| | | (aa) |
| | | Homo sapiens |
| 2 | GAATCTAAGTACGGACCGCCCTGCCCCCCTTGCCCT | spacer |
| | | (IgG4hinge) |
| | | (nt) |
| | | homo sapiens |
| 3 | ESKYGPPCPPCPGQPREPQVYTLPPSQEEMTKNQVSLTCLVKGFYPSDI | Hinge-CH3 |
| | AVEWESNGQPENNYKTTPPVLDSDGSFFLYSRLTVDKSRWQEGNVFSCS | spacer |
| | VMHEALHNHYTQKSLSLSLGK | Homo sapiens |
| 4 | ESKYGPPCPPCPAPEFLGGPSVFLFPPKPKDTLMISRTPEVTCVVVDVS | Hinge-CH2- |
| | QEDPEVQFNWYVDGVEVHNAKTKPREEQFNSTYRVVSVLTVLHQDWLNG | CH3 spacer |
| | KEYKCKVSNKGLPSSIEKTISKAKGQPREPQVYTLPPSQEEMTKNQVSL | Homo sapiens |
| | TCLVKGFYPSDIAVEWESNGQPENNYKTTPPVLDSDGSFFLYSRLTVDK | |
| | SRWQEGNVFSCSVMHEALHNHYTQKSLSLSLGK | |
| 5 | RWPESPKAQASSVPTAQPQAEGSLAKATTAPATTRNTGRGGEEKKKEKE | IgD-hinge-Fc |
| | KEEQEERETKTPECPSHTQPLGVYLLTPAVQDLWLRDKATFTCFVVGSD | Homo sapiens |
| | LKDAHLTWEVAGKVPTGGVEEGLLERHSNGSQSQHSRLTLPRSLWNAGT | |
| | SVTCTLNHPSLPPQRLMALREPAAQAPVKLSLNLLASSDPPEAASWLLC | |
| | EVSGFSPPNILLMWLEDQREVNTSGFAPARPPPQPGSTTFWAWSVLRVP | |
| | APPSPQPATYTCVVSHEDSRTLLNASRSLEVSYVTDH | |
| 6 | LEGGGEGRGSLLTCGDVEENPGPR | T2A |
| | | artificial |
| 7 | MLLLVTSLLLCELPHPAFLLIPRKVCNGIGIGEFKDSLSINATNIKHFK | tEGFR |
| | NCTSISGDLHILPVAFRGDSFTHTPPLDPQELDILKTVKEITGFLLIQA | artificial |
| | WPENRTDLHAFENLEIIRGRTKQHGQFSLAVVSLNITSLGLRSLKEISD | |
| | GDVIISGNKNLCYANTINWKKLFGTSGQKTKIISNRGENSCKATGQVCH | |
| | ALCSPEGCWGPEPRDCVSCRNVSRGRECVDKCNLLEGEPREFVENSECI | |
| | QCHPECLPQAMNITCTGRGPDNCIQCAHYIDGPHCVKTCPAGVMGENNT | |
| | LVWKYADAGHVCHLCHPNCTYGCTGPGLEGCPTNGPKIPSIATGMVGAL | |
| | LLLLVVALGIGLFM | |
| 8 | FWVLVVVGGVLACYSLLVTVAFIIFWV | CD28 (amino |
| | | acids 153-179 of |
| | | Accession |
| | | No. P10747) |
| | | Homo sapiens |
| 9 | IEVMYPPPYLDNEKSNGTIIHVKGKHLCPSPLFPGPSKP | CD28 (amino |
| | FWVLVVVGGVLACYSLLVTVAFIIFWV | acids 114-179 of |
| | | Accession |
| | | No. P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPEMGGKP | CD3 zeta |
| | RRKNPQEGLYNELQKDKMAEAYSEIGMKGERRRGKGHDGLYQGLSTATK | Homo sapiens |
| | DTYDALHMQALPPR | |
| 14 | RVKFSRSAEPPAYQQGQNQLYNELNLGRREEYDVLDKRRGRDPEMGGKP | CD3 zeta |
| | RRKNPQEGLYNELQKDKMAEAYSEIGMKGERRRGKGHDGLYQGLSTATK | Homo sapiens |
| | DTYDALHMQALPPR | |
| 15 | RVKFSRSADAPAYKQGQNQLYNELNLGRREEYDVLDKRRGRDPEMGGKP | CD3 zeta |
| | RRKNPQEGLYNELQKDKMAEAYSEIGMKGERRRGKGHDGLYQGLSTATK | Homo sapiens |
| | DTYDALHMQALPPR | |
| 16 | RKVCNGIGIGEFKDSLSINATNIKHFKNCTSISGDLHILPVAFRGDSFT | tEGFR |
| | HTPPLDPQELDILKTVKEITGFLLIQAWPENRTDLHAFENLEIIRGRTK | artificial |
| | QHGQFSLAVVSLNITSLGLRSLKEISDGDVIISGNKNLCYANTINWKKL | |
| | FGTSGQKTKIISNRGENSCKATGQVCHALCSPEGCWGPEPRDCVSCRNV | |
| | SRGRECVDKCNLLEGEPREFVENSECIQCHPECLPQAMNITCTGRGPDN | |
| | CIQCAHYIDGPHCVKTCPAGVMGENNTLVWKYADAGHVCHLCHPNCTYG | |
| | CTGPGLEGCPTNGPKIPSIATGMVGALLLLLVVALGIGLFM | |
| 17 | ACACGGCCTCGTGTATTACTGT | exemplary |
| | | degenerate |
| | | sequence of |
| | | the V region |
| | | of IGH |
| 18 | ACCTGAGGAGACGGTGACC | exemplary |
| | | degenerate |
| | | sequence of |
| | | the J region |
| | | of IGH |
| 19 | EGRGSLLTCGDVEENPGP | T2A |
| | | artificial |
| 20 | GSGATNFSLLKQAGDVEENPGP | P2A |
| 21 | ATNFSLLKQAGDVEENPGP | P2A |
| 22 | QCTNYALLKLAGDVESNPGP | E2A |
| 23 | VKQTLNFDLLKLAGDVESNPGP | F2A |
| 24 | GSTSGSGKPGSGEGSTKG | Linker |
| 25 | gacatccaga tgacccagac cacctccagc ctgagcgcca | Sequence |
| | gcctgggcga ccgggtgacc atcagctgcc gggccagcca | encoding |
| | ggacatcagc aagtacctga actggtatca gcagaagccc | scFv |
| | gacggcaccg tcaagctgct gatctaccac accagccggc | |
| | tgcacagcgg cgtgcccagc cggtttagcg gcagcggctc | |
| | cggcaccgac tacagcctga ccatctccaa cctggaacag | |
| | gaagatatcg ccacctactt ttgccagcag ggcaacacac | |
| | tgccctacac ctttggcggc ggaacaaagc tggaaatcac | |
| | cggcagcacc tccggcagcg gcaagcctgg cagcggcgag | |
| | ggcagcacca agggcgaggt gaagctgcag gaaagcggcc | |
| | ctggcctggt ggcccccagc cagagcctga gcgtgacctg | |
| | caccgtgagc ggcgtgagcc tgcccgacta cggcgtgagc | |
| | tggatccggc agccccccag gaagggcctg gaatggctgg | |
| | gcgtgatctg gggcagcgag accacctact acaacagcgc | |
| | cctgaagagc cggctgacca tcatcaagga caacagcaag | |
| | agccaggtgt tcctgaagat gaacagcctg cagaccgacg | |
| | acaccgccat ctactactgcgccaagcact actactacgg | |
| | cggcagctac gccatggact actggggcca gggcaccagc | |
| | gtgaccgtga gcagc | |
| 26 | X₁PPX₂P | Hinge |
| | X₁ is glycine, cysteine or arginine | |
| | X₂ is cysteine or threonine | |
| 27 | Glu Pro Lys Ser Cys Asp Lys Thr His Thr Cys Pro | Hinge |
| | Pro Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEPK | Hinge |
| | SCDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro | Hinge |
| | Cys Pro | |
| 35 | RASQDISKYLN | FMC63 CDR L1 |
| 36 | SRLHSGV | FMC63 CDR L2 |
| 37 | GNTLPYTFG | FMC63 CDR L3 |
| 38 | DYGVS | FMC63 CDR H1 |
| 39 | VIWGSETTYYNSALKS | FMC63 CDR H2 |
| 40 | YAMDYWG | FMC63 CDR H3 |
| 41 | EVKLQESGPGLVAPSQSLSVTCTVSGVSLPDYGVSWIRQPPRKGLEWLG | FMC63 VH |
| | VIWGSETTYYNSALKSRLTIIKDNSKSQVFLKMNSLQTDDTAIYYCAKH | |
| | YYYGGSYAMDYWGQGTSVTVSS | |
| 42 | DIQMIQTTSSLSASLGDRVTISCRASQDISKYLNWYQQKPDGIVKLLIY | FMC63 VL |
| | HTSRLHSGVPSRFSGSGSGTDYSLTISNLEQEDIATYFCQQGNTLPYTF | |
| | GGGTKLEIT | |
| 43 | DIQMIQTTSSLSASLGDRVTISCRASQDISKYLNWYQQKPDGIVKLLIY | FMC63 scFv |
| | HTSRLHSGVPSRFSGSGSGTDYSLTISNLEQEDIATYFCQQGNTLPYTF | |
| | GGGTKLEITGSTSGSGKPGSGEGSTKGEVKLQESGPGLVAPSQSLSVTC | |
| | TVSGVSLPDYGVSWIRQPPRKGLEWLGVIWGSETTYYNSALKSRLTIIK | |
| | DNSKSQVFLKMNSLQTDDTAIYYCAKHYYYGGSYAMDYWGQGTSVTVSS | |
| 44 | KASQNVGTNVA | SJ25C1 CDR |
| | | L1 |
| 45 | SATYRNS | SJ25C1 CDR |
| | | L2 |
| 46 | QQYNRYPYT | SJ25C1 CDR |
| | | L3 |
| 47 | SYWMN | SJ25C1 CDR |
| | | H1 |
| 48 | QIYPGDGDTNYNGKFKG | SJ25C1 CDR |
| | | H2 |
| 49 | KTISSVVDFYFDY | SJ25C1 CDR |
| | | H3 |
| 50 | EVKLQQSGAELVRPGSSVKISCKASGYAFSSYWMNWVKQRPGQGLEWIG | SJ25C1 VH |
| | QIYPGDGDTNYNGKFKGQATLTADKSSSTAYMQLSGLTSEDSAVYFCAR | |
| | KTISSVVDFYFDYWGQGTTVTVSS | |
| 51 | DIELTQSPKFMSTSVGDRVSVTCKASQNVGTNVAWYQQKPGQSPKPLIY | SJ25C1 VL |
| | SATYRNSGVPDRFTGSGSGTDFTLTITNVQSKDLADYFCQQYNRYPYTS | |
| | GGGTKLEIKR | |
| 52 | GGGGSGGGGSGGGGS | Linker |
| 53 | EVKLQQSGAELVRPGSSVKISCKASGYAFSSYWMNWVKQRPGQGLEWIG | SJ25C1 scFv |
| | QIYPGDGDTNYNGKFKGQATLTADKSSSTAYMQLSGLTSEDSAVYFCAR | |
| | KTISSVVDFYFDYWGQGTTVTVSSGGGGSGGGGSGGGGSDIELTQSPKF | |
| | MSTSVGDRVSVTCKASQNVGTNVAWYQQKPGQSPKPLIYSATYRNSGVP | |
| | DRFTGSGSGTDFTLTITNVQSKDLADYFCQQYNRYPYTSGGGTKLEIKR | |
| 54 | HYYYGGSYAMDY | FMC63 CDR H3 |
| 55 | HTSRLHS | FMC63 CDR L2 |
| 56 | QQGNTLPYT | FMC63 CDR L3 |

1-118. (canceled)

119. A method of treating a subject having or suspected of having a chronic lymphocytic leukemia (CLL), the method comprising administering to the subject a dose of cells expressing a chimeric antigen receptor (CAR) that specifically binds to a target antigen expressed by the CLL,
wherein, prior to the administration, the subject has been preconditioned with a lymphodepleting therapy comprising the administration of fludarabine.

120. The method of claim 119, wherein the dose comprises (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cells/kg.

121. The method of claim 119, wherein the dose comprises (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.

122. The method of claim 119, wherein, at or prior to the administration of the dose of cells:
the subject is or has been identified as having one or more cytogenetic abnormalities associated with high-risk CLL;
the subject is or has been identified as having high-risk CLL; and/or
the subject is or has been identified as having extramedullary disease; and/or
the subject is or has been identified as having central nervous system (CNS) disease.

123. The method of claim 121, wherein, at or prior to the administration of the dose of cells:
the subject is or has been identified as having one or more cytogenetic abnormalities associated with high-risk CLL;
the subject is or has been identified as having high-risk CLL; and/or
the subject is or has been identified as having extramedullary disease; and/or
the subject is or has been identified as having central nervous system (CNS) disease.

124. The method of claim 119, wherein the CLL is a relapsed/refractory (R/R) CLL and/or a high-risk CLL.

125. The method of claim 121, wherein the CLL is a relapsed/refractory (R/R) CLL and/or a high risk CLL.

126. The method of claim 119, wherein, prior to the administration of the dose of cells, the subject has been treated with two or more therapies for the CLL, other than the lymphodepleting therapy and/or other than another dose of cells expressing the CAR.

127. The method of claim 119, wherein, prior to the administration of the dose of cells, the subject has been treated for the CLL with an inhibitor of Btk.

128. The method of claim 119, wherein, prior to the administration of the dose of cells, the subject has been treated for the CLL with ibrutinib.

129. The method of claim 121, wherein, prior to the administration of the dose of cells, the subject has been treated for the CLL with ibrutinib.

130. The method of claim 119, wherein, prior to the administration of the dose of cells, the subject has been treated for the CLL with a monoclonal antibody that specifically binds to an antigen expressed by, or previously expressed by, cells of the CLL.

131. The method of claim 119, wherein, prior to the administration of the dose of cells, the subject has been treated for the CLL with venetoclax, a combination therapy comprising fludarabine and rituximab, radiation therapy and/or hematopoietic stem cell transplantation (HSCT).

132. The method of claim 119, wherein prior to administration of the dose of cells, the subject has been treated for the CLL with two or more therapies selected from among venetoclax, a combination therapy comprising fludarabine and rituximab, radiation therapy and hematopoietic stem cell transplantation (HSCT).

133. The method of claim 119, wherein, at or immediately prior to the time of the administration of the dose of cells, the subject has relapsed following remission after treatment with, or become refractory to, one or more prior therapies for the CLL.

134. The method of claim 133, wherein the one or more prior therapies is or comprises ibrutinib.

135. The method of claim 119, further comprising, prior to the administration of the cell dose, administering the lymphodepleting therapy to the subject.

136. The method of claim 119, wherein the lymphodepleting therapy further comprises another chemotherapeutic agent other than the fludarabine, wherein the other chemotherapeutic agent is cyclophosphamide.

137. The method of claim 135, wherein the lymphodepleting therapy further comprises administering another chemotherapeutic agent other than fludarabine, wherein the other chemotherapeutic agent is cyclophosphamide.

138. The method of claim 119, wherein the lymphodepleting therapy is initiated at a time that is at least at or about 48 hours prior to or is between at or about 48 and at or about 96 hours prior to the administration of the cells.

139. The method of claim 135, wherein administration of the lymphodepleting therapy is initiated at a time that is at least at or about 48 hours prior to or is between at or about 48 and at or about 96 hours prior to the administration of the cells.

140. The method of claim 119, wherein the administration of the cell dose and/or the lymphodepleting therapy is carried out via outpatient delivery.

141. The method of claim 119, wherein the dose of cells comprises a defined ratio of CD4+ cells expressing the CAR to CD8+ cells expressing the CAR and/or of CD4+ cells to CD8+ cells, which is between approximately 1:3 and approximately 3:1.

142. The method of claim 119, wherein the dose of cells comprises a defined ratio of CD4+ cells expressing the CAR to CD8+ cells expressing the CAR and/or of CD4+ cells to CD8+ cells, which is approximately 1:1.

143. The method of claim 119, wherein:
at least 50% of subjects treated according to the method achieve complete remission (CR) and/or objective response (OR); and/or
the subject exhibits CR, OR, or lymph nodes of less than at or about 20 mm in size, within 1 month of the administration of the dose of cells; and/or
wherein a malignant immunoglobulin heavy chain locus (IGH) and/or an index clone of the CLL is not detected in the bone marrow of the subject or in the bone marrow of greater than 50% of subjects treated according to the methods.

144. The method of claim 119, wherein:
at least 50% of subjects treated according to the method achieve complete remission (CR), exhibit progression-free survival (PFS) and/or overall survival (OS) of greater than 12 months;
on average, subjects treated according to the method exhibit a median PFS or OS of greater than at or about 6 months; and/or
the subject exhibits PFS or OS following therapy for at least at or about 6 months.

145. The method of claim 119, wherein the antigen is a B cell antigen.

146. The method of claim 119, wherein the antigen is CD19.

147. The method of claim 119, wherein the CAR comprises an scFv specific for the antigen, a transmembrane domain, a cytoplasmic signaling domain derived from a costimulatory molecule, which is a 4-1BB, and a cytoplasmic signaling domain derived from a primary signaling ITAM-containing molecule, which is a CD3zeta.

148. A method of treating a subject having a non-Hodgkin lymphoma (NHL), the method comprising administering to the subject a dose of cells expressing a chimeric antigen receptor (CAR) that specifically binds to a target antigen expressed by the NHL, wherein:
the dose comprises a defined ratio of CD4⁺ cells expressing the CAR to CD8⁺ cells expressing the CAR and/or of CD4⁺ cells to CD8⁺ cells, which ratio is between approximately 1:3 and approximately 3:1, and
wherein, prior to the administration, the subject has been preconditioned with a lymphodepleting therapy comprising the administration of fludarabine.

149. The method of claim 148, wherein the dose comprises (a) at or about 2×10⁵ of the cells per kilogram body weight of the subject (cells/kg); (b) at or about 2×10⁶ of the cells/kg, (c) no more than at or about 2×10⁶ of the cells/kg, (d) no more than at or about 2×10⁵ of the cells/kg and/or (e) between at or about 2×10⁵ of the cells/kg and at or about 2×10⁶ of the cell s/kg.

150. The method of claim 148, wherein the dose comprises (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.

151. A method of prognosis or staging, the method comprising detecting the presence or absence of a malignant immunoglobulin heavy chain locus (IGH) sequence in a sample from a subject having a B cell malignancy, said subject having previously received administration of a cell therapy comprising a dose or composition of genetically engineered cells expressing a recombinant receptor for treating the B cell malignancy, wherein detecting the presence or absence of the malignant IGH sequence determines the prognosis of the subject in response to the cell therapy.

152. The method of claim 151, wherein if the malignant IGH sequence is detected administering to the subject a further dose of the cell therapy, administering to the subject a higher dose of the cell therapy, administering to the subject a different cell therapy, and/or administering to the subject an alternative therapeutic agent for treating the B cell malignancy.

153. A method of predicting durability of response to a cell therapy, the method comprising detecting the presence or absence of a malignant immunoglobulin heavy chain locus (IGH) sequence in a sample from a subject having a B cell malignancy, said subject having previously received administration of a cell therapy comprising a dose or composition of genetically engineered cells expressing a recombinant receptor for treating the B cell malignancy, wherein the presence or absence of the malignant IGH sequence predicts the durability of response to the cell therapy.

154. The method of claim 153, wherein the detecting the presence or absence of the malignant IGH sequence is carried out within or within about or about 16 weeks after initiation of the cell therapy.

155. The method of claim 153, wherein:
if the malignant IGH sequence is not detected, the subject is predicted to exhibit or likely to exhibit a durable response to the cell therapy and/or to be at a low or relatively low risk of relapse within a certain period of time and/or to have a high likelihood of exhibiting progression free survival for at least a certain period of time; and
if the malignant IGH sequence is detected, the subject is predicted to exhibit or likely to exhibit a response to the cell therapy that is not durable and/or to be at a high or relatively high risk of relapse within a certain period of time and/or to have a low likelihood of exhibiting progression free survival for at least a certain period of time.

156. The method of claim 153, wherein if the malignant IGH sequence is detected administering to the subject a further dose of the cell therapy, administering to the subject a higher dose of the cell therapy, administering to the subject a different cell therapy, and/or administering to the subject an alternative therapeutic agent for treating the B cell malignancy.

157. The method of claim 153, wherein the presence or absence of the malignant IGH sequence is determined by IGH sequencing.

158. An article of manufacture comprising one or more dose of a cell therapy, each dose comprising cells expressing a chimeric antigen receptor (CAR), and instructions for administering the cell therapy, wherein:
the instructions specify the dose of cells is to be administered to a subject having a chronic lymphocytic leukemia (CLL) or a non-Hodgkin lymphoma (NHL); and
the instructions specify administration of a number of CAR-expressing or a number of cells, or specify administration of an amount or volume of one or more formulations corresponding to or containing said specified number of cells, wherein the specified number of cells to be administered comprises a number to administer a dose of cells comprising (a) at or about 1×10⁷ total cells or total CAR-expressing cells; (b) at or about 1.5×10⁸ total cells or total CAR-expressing cells, (c) no more than at or about 1×10⁷ total cells or total CAR-expressing cells, (d) no more than at or about 1.5×10⁸ total cells or total CAR-expressing cells and/or (e) between at or about 1×10⁷ total cells or total CAR-expressing cells and at or about 1.5×10⁸ total cells or total CAR-expressing cells.
